SEQUENCE LISTING (75 sequences)

SEQ ID NO: 1 - DNA, 1691 bp, Homo sapiens
aacttcagtttgttggctgcggcagcaggtagcaaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 2 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 3 - DNA, 1912 bp, Homo sapiens
ttttttttcttccctctagtgggcggggcagaggagttagccaagatgtgactttgaaaccctcagcgtctcagtgcccttttgttctaaacaaagaattttgtaattggttctaccaaagaaggatataatgaagtcactatgggaaaagatggggaggagagttgtaggattctacattaattctcttgtgcccttagcccactacttcagaatttcctgaagaaagcaagcctgaattggttttttaaattgctttaaaaattttttttaactgggttaatgcttgctgaattggaagtgaatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 4 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 5 - DNA, 1083 bp, Homo sapiens
atggacccagaagaaacttcagtttatttggattattactatgctacgagcccaaactctgacatcagggagacccactcccatgttccttacacctctgtcttccttccagtcttttacacagctgtgttcctgactggagtgctggggaaccttgttctcatgggagcgttgcatttcaaacccggcagccgaagactgatcgacatctttatcatcaatctggctgcctctgacttcatttttcttgtcacattgcctctctgggtggataaagaagcatctctaggactgtggaggacgggctccttcctgtgcaaagggagctcctacatgatctccgtcaatatgcactgcagtgtcctcctgctcacttgcatgagtgttgaccgctacctggccattgtgtggccagtcgtatccaggaaattcagaaggacagactgtgcatatgtagtctgtgccagcatctggtttatctcctgcctgctggggttgcctactcttctgtccagggagctcacgctgattgatgataagccatactgtgcagagaaaaaggcaactccaattaaactcatatggtccctggtggccttaattttcaccttttttgtccctttgttgagcattgtgacctgctactgttgcattgcaaggaagctgtgtgcccattaccagcaatcaggaaagcacaacaaaaagctgaagaaatctataaagatcatctttattgtcgtggcagcctttcttgtctcctggctgcccttcaatactttcaagttcctggccattgtctctgggttgcggcaagaacactatttaccctcagctattcttcagcttggtatggaggtgagtggacccttggcatttgccaacagctgtgtcaaccctttcatttactatatcttcgacagctacatccgccgggccattgtccactgcttgtgcccttgcctgaaaaactatgactttgggagtagcactgagacatcagatagtcacctcactaaggctctctccaccttcattcatgcagaagattttgccaggaggaggaagaggtctgtgtcactctaa

SEQ ID NO: 6 - protein, 360 aa, Homo sapiens
MDPEETSVYLDYYYATSPNSDIRETHSHVPYTSVFLPVFYTAVFLTGVLGNLVLMGALHFKPGSRRLIDIFIINLAASDFIFLVTLPLWVDKEASLGLWRTGSFLCKGSSYMISVNMHCSVLLLTCMSVDRYLAIVWPVVSRKFRRTDCAYVVCASIWFISCLLGLPTLLSRELTLIDDKPYCAEKKATPIKLIWSLVALIFTFFVPLLSIVTCYCCIARKLCAHYQQSGKHNKKLKKSIKIIFIVVAAFLVSWLPFNTFKFLAIVSGLRQEHYLPSAILQLGMEVSGPLAFANSCVNPFIYYIFDSYIRRAIVHCLCPCLKNYDFGSSTETSDSHLTKALSTFIHAEDFARRRKRSVSL

SEQ ID NO: 7 - DNA, 1083 bp, Mus musculus
atggaaccagcaacagccctgctgattgtggattactatgactacacaagcccagatcctcctttcctggagactccctcccacctgtcctacacatctgtcttcctccctatcttttacacagttgtattcttgactggagtggtggggaatttcatcctcatgatagctctgcatttcaaacgcggcaaccgaagattgatcgacatctttatcatcaacctggctgcctctgacttcattttccttgtcacagtgcctctttggatggataaggaagcctctctaggactatggaggactggctctttcctgtgcaaaggcagctcctatgtgatctccgtgaacatgcactgtagtgtcttcttgctcacatgcatgagcatggaccgttacctggctatcatgcacccagccttagccaagagattacgaaggagaagctctgcatatgcagtgtgtgccgtcgtctggatcatctcatgcgtcctggggttgcccactcttctgtccagggagctcactcacattgaaggcaaaccatactgtgcagagaagaaacccacgtccttaaaactgatgtggggcctggtagccttgattaccacctttttcgtccccctcctgagcattgtgacctgctactgttgcatcacaaggaggctgtgtgctcattaccagcagtcgggaaagcataacaagaaactaaagaagtccataaagatcgttattattgcggtggcggccttcaccgtctcctgggtaccctttaacactttcaagctcctagccattgtttcagggttccagccagaaggcctttttcactccgaggctttgcagctggccatgaatgtgactgggcccttggcctttgccagcagctgtgtcaaccctctcatttactatgtctttgacagctatatccgccgggccattgtacgttgtctgtgcccttgtctgaagacccacaactttgggagcagcactgagacatcggacagtcacctcactaaggctctttccaacttcattcatgcagaggatttcatccggcggaggaagagatctgtgtcactctaa

SEQ ID NO: 8 - protein, 360 aa, Mus musculus
MEPATALLIVDYYDYTSPDPPFLETPSHLSYTSVFLPIFYTVVFLTGVVGNFILMIALHFKRGNRRLIDIFIINLAASDFIFLVTVPLWMDKEASLGLWRTGSFLCKGSSYVISVNMHCSVFLLTCMSMDRYLAIMHPALAKRLRRRSSAYAVCAVVWIISCVLGLPTLLSRELTHIEGKPYCAEKKPTSLKLMWGLVALITTFFVPLLSIVTCYCCITRRLCAHYQQSGKHNKKLKKSIKIVIIAVAAFTVSWVPFNTFKLLAIVSGFQPEGLFHSEALQLAMNVTGPLAFASSCVNPLIYYVFDSYIRRAIVRCLCPCLKTHNFGSSTETSDSHLTKALSNFIHAEDFIRRRKRSVSL

SEQ ID NO: 9 - DNA, 576 bp, Encephalomyocarditis virus
ccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataa

SEQ ID NO: 10 - DNA, 192 bp, Cricket paralysis virus
aaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacct

SEQ ID NO: 11 - DNA, 383 bp, Hepatitis C type 1a
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaac

SEQ ID NO: 12 - DNA, 461 bp, Foot and Mouth Disease Virus type C
agcaggtttccccaactgacacaaaacgtgcaacttgaaactccgcctggtctttccaggtctagaggggtaacactttgtactgtgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttacaattaatgaccct

SEQ ID NO: 13 - DNA, 584 bp, Attenuated Hepatitis A Virus
taattcctgcaggttcagggttcttaaatctgtttctctataagaacactcatttttcacgctttctgtcttctttcttccagggctctccccttgccctaggctctggccgttgcgcccggcggggtcaactccatgattagcatggagctgtaggagtctaaattggggacacagatgtttggaacgtcaccttgcagtgttaacttggctttcatgaatctctttgatcttccacaaggggtaggctacgggtgaaacctcttaggctaatacttctatgaagagatgccttggatagggtaacagcggcggatattggtgagttgttaagacaaaaaccattcaacgccggaggactgactctcatccagtggatgcattgagtggattgactgtcagggctgtctttaggcttaattccagacctctctgtgcttagggcaaacatcatttggccttaaatgggattctgtgagaggggatccctccattgacagctggactgttctttggggccttatgtggtgtttgcctctgaggtactcaggggcatttaggtttttcctcattcttaaataata

SEQ ID NO: 14 - DNA, 312 bp, Polio virus type 1 Mahoney
atgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggcc

SEQ ID NO: 15 - DNA, 742 bp, Polio Virus type 3 Leon
ttaaaacagctctggggttgttcccaccccagaggcccacgtggcggctagtacactggtatcacggtacctttgtacgcctgttttatactccctcccccgcaacttagaagcatacaattcaagctcaataggagggggtgcaagccagcgcctccgtgggcaagcactactgtttccccggtgaggccgcatagactgttcccacggttgaaagtggccgatccgttatccgctcatgtacttcgagaagcctagtatcgctctggaatcttcgacgcgttgcgctcagcactcaaccccggagtgtagcttgggccgatgagtctggacagtccccactggcgacagtggtccaggctgcgctggcggcccacctgtggcccaaagccacgggacgctagttgtgaacagggtgtgaagagcctattgagctacatgagagtcctccggcccctgaatgcggctaatcctaaccatggagcaggcagctgcaacccagcagccagcctgtcgtaacgcgcaagtccgtggcggaaccgactactttgggtgtccgtgtttccttttattcttgaatggctgcttatggtgacaatcatagattgttatcataaagcgagttggattggccatccagtgtgaatcagattaattactcccttgtttgttggatccactcccgaaacgttttactccttaacttattgaaattgtttgaagacaggatttcagtgtcaca

SEQ ID NO: 16 - DNA, 494 bp, Avian encephalomyelitis virus
tttgaaagaggcctccggagtgtccggaggctctctttcgacccaacccatactggggggtgtgtgggaccgtacctggagtgcacggtatatatgcattcccgcatggcaagggcgtgctaccttgccccttgacgcatggtatgcgtcatcatttgccttggttaagccccatagaaacgaggcgtcacgtgccgaaaatccctttgcgtttcacagaaccatcctaaccatgggtgtagtatgggaatcgtgtatggggatgattaggatctctcgtagagggataggtgtgccattcaaatccagggagtactctggctctgacattgggacatttgatgtaaccggacctggttcagtatccgggttgtcctgtattgttacggtgtatccgtcttggcacactgaaagggtatttttgggtaatcctttcctactgcctgatagggtggcgtgcccggccacgagagattaagggtagcaatttaaac

SEQ ID NO: 17 - DNA, 373 bp, Classical swine fever virus
gtatacgaggttagttcattctcgtatacacgattggacaaatcaaaattataatttggttcagggcctccctccagcgacggccgaactgggctagccatgcccatagtaggactagcaaaacggagggactagccatagtggcgagctccctgggtggtctaagtcctgagtacaggacagtcgtcagtagttcgacgtgagcagaagcccacctcgagatgctacgtggacgagggcatgccaagacacaccttaaccctagcgggggtcgctagggtgaaatcacaccacgtgatgggagtacgacctgatagggcgctgcagaggcccactattaggctagtataaaaatctctgctgtacatggcac

SEQ ID NO: 18 - DNA, 712 bp, Equine rhinitis A virus
gagaggagcccgttttcgggcacttgtctcctaaacaatgttggcgcgcatttgcgcgccccccccctttttcagccccctgtcattgactggtcgaagcgttcgcaataagactggtcgtcacttggctgttctatcgtttcaggctttagcgcgcccttgcgcggcgggccgtcaagcccgtgcgctgtatagcgccaggtaaccggacagcggcgtgctggattttcccggtgccattgctctggatggtgtcaccaagctgacaaatgcggagtgaacctcacaaagcgacacgcctgtggtagcgctgcccaaaagggagcggaactccccgccgaggcggtcctctctggccaaaagcccagcgttgatagcgccttttgggatgcaggaaccccacctgccaggtgtgaagtggagtgagcggatctccaatttggtctgttctgaactacaccatttactgctgtgaagaatgccctggaggcaagctggttacagccctgaccaggccctgcccgtgactctcgaccggcgcagggtcaaaaattgtctaagcagcagcaggaacgcgggagcgtttcttttccttttgtactgacatgatggcggcgtctaaggtgtatagagtttgcgagcagactctgctggcaggtgccgttcgcatgatggacaaattcttgcaaaagagaactgtttttgtccccca

SEQ ID NO: 19 - DNA, 583 bp, Apoptotic protease activating factor virus
cggcttgaggcagagaccaggaggcagctagaggagcagacgtctcactccgctcgcggaagggtgtgagaggggtgtgtgggggtcggcagcgaggggtgtgtgccatcagccaccggcgacgatctgagacagtcgcagcggctttccgagcggcgtccgcttcccgcccgggcagctcccgccagaggggtgaagcggcgactggagtggccgtgcttttgtgccctgggtcccggtaccctcccctggtgcggcccgaggcaagcccaccgaggtgaccacccctcgacgccgcttggagatcccgggcatccaccctgcgccccgagcagctgatacccagggaggtgtcaggacctgcccggggcgcggggtcgccggaagccaggcgggagccccggctgctttctggcaatctagtctcataagtgaccctccctgggctgctttctttcgattatcatcagtgaccctaccccggctgctcttcccagcacaactccggtgcaaaggcttgggcatcctggtgctttgcctctagcccatgctccacagcgaggagagagaaaaccctgaggca

SEQ ID NO: 20 - DNA, 1137 bp, Homo sapiens
aaaaaataaaaccctcccccaccacctccttctccccacccctcgccgcaccacacacagcgcgggcttctagcgctcggcaccggcgggccaggcgcgtcctgccttcatttatccagcagcttttcggaaaatgcatttgctgttcggagtttaatcagaagacgattcctgcctccgtccccggctccttcatcgtcccatctcccctgtctctctcctggggaggcgtgaagcggtcccgtggatagagattcatgcctgtgtccgcgcgtgtgtgcgcgcgtataaattgccgagaaggggaaaacatcacaggacttctgcgaataccggactgaaaattgtaattcatctgccgccgccgctgccaaaaaaaaactcgagctcttgagatctccggttgggattcctgcggattgacatttctgtgaagcagaagtctgggaatcgatctggaaatcctcctaatttttactccctctccccccgactcctgattcattgggaagtttcaaatcagctataactggagagtgctgaagattgatgggatcgttgccttatgcatttgttttggttttacaaaaaggaaacttgacagaggatcatgctgtacttaaaaaatacaagtaagtctcgcacaggaaattggtttaatgtaactttcaatggaaacctttgagattttttacttaaagtgcattcgagtaaatttaatttccaggcagcttaatacattgtttttagccgtgttacttgtagtgtgtatgccctgctttcactcagtgtgtacagggaaacgcacctgattttttacttattagtttgttttttctttaacctttcagcatcacagaggaagtagactgatattaacaatacttactaataataacgtgcctcatgaaataaagatccgaaaggaattggaataaaaatttcctgcgtctcatgccaagagggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaagg

SEQ ID NO: 21 - DNA, 319 bp, Aplysia californica
ctttaccgacaacgtccagcggtccagtcgagaagtaagtagtccgcgctgcgagcataactctaccagagaaacgcgcactttattggtcaagacagacggaagccggcggttggacgcaaaggttgactcggaagctgagagattgttgagtaacacgcagaagtcttgaaagtatcaagtattgaacatatttcaagggacttggtttcggtgaagtcgtcaattccttttatcgtcaacgtttccacagccctcagaatagaaatttccaacaagccaaagcctacgtaatgaagcgccccaataaccggccgac

SEQ ID NO: 22 - DNA, 252 bp, Homo sapiens
ggcctcagtcaggcctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggcggcggcggcggcggcggctgggcctcgagcgcccgcagcccacctctcgggggcgggctcccggcgctagcagggctgaagagaaca

SEQ ID NO: 23 - DNA, 193 bp, Homo sapiens
ctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccggc

SEQ ID NO: 24 - DNA, 1196 bp, Mus musculus
actgggatggatactggagaaggaatgcaggcttaacaagtgatcgctgctgtctaggattttgagtctttttcggagaaccttgacttccgttcccagcccatgtctgctgtgccgaactccagaggaaccagaaatctccggggtctaccttggggcgtccccaatctccacctctgggctccagtaacgaggactctgcaataccccctagccccctggccaagacaaccgaacttgttccgtggatatttgggatcctccacctgccaaacctgagcgattttttttgtactgcgcccccacccccaatgattctgcccctcctccagctgttgcagcgtggaaaaggggaaacaaatcaccgggggggatttttttcgtctatttttatttttcgcacttgctgggaatggtgaagtgcttcttgtgaatgattctagccaaaggatgctcttcatttcctgctttctatggagacctcagtgttgagtttgcctctgctggaactgcgtctaccaccttctctaccttccaaggtctttgcctctatctacaacctggcattgtctgtgggtccatgaaggcttggatgaccttagagggaaggtctgggagtccaccctcatagactaagcagcaatggctgggcatattttaagccgcattttaacatgggtcaagccatcagtagaaggcaagtgctaagactaaagacttatttgaattttatttaaattagatggactgggccttggccaatttccatgcaagaaaaagtatatttcattttctaggcacaacttctgagtgtcagatacttgctgtctttgagtcttgtggcgtcatcaccggacagcatcccagacagacttccagatttgaacatctaccccccaacacgtaggtgtatgggagaccacatcatttcatgacttatgtttgaggaacactaggctgttgtctagacgaggcaagctctggaaagcaacgccgagtctctgagaagagggagcataggctgtgctgatttaaaaacagaaaatgcaaagttggactgaaaatatcccacgtcttctaagcaatctgcttaaggcttccaaacttaccttaatttggtaagaaaataagctgccctatttttctttcttcttctcttacaactggaagcagccatttccccaaaccaccaccat

SEQ ID NO: 25 - DNA, 1167 bp, Homo sapiens
gcgcgggaggaggagaagcagtggggaggcgcagccgctcacctgcggggcagggcgcggaggagggacccggtgcgcgctctcgggccgaggaaccaggacgcgcccggagcctcgcacgcggccaagctcgggcgtcccctcccctcggccgggcgaactcaaggggcgcagctctttgctttgacagagctggccggcggaggcgtgcagagcggcgagccggcgagccaggctgagaaactcgacccgggaacaaagaggggtcggactgagtgtgtgtgtcggctcgagctccgggagaggcatttgcccgaggccccgctgtgactccccgagactccgcagtgccctccactgggagtccccgcgcttgccggaaaaactttattcttggcaaacttctctttctcttcccctcctcctcggcccccatcttctgctcctcctccttctctagcagattaaatgagcctcgagaagaaaaaccgaagcgaaagggaagaaaataagaagatctaaaacggacatctccagcgtgggtggctcctttttctttttctttttttcccacccttcaggaagtggacgtttcgttatcttctgatccttgcaccttcttttggggcaaacggggcccttctgcccagatcccctctcttttctcggaaaacaaactactaagtcggcatccggggtaactacagtggagagggtttccgcggagacgcgccgccggaccctcctctgcactttggggaggcgtgctccctccagaaccggcgttctccgcgcgcaaatcccggcgacgcggggtcgcggggtggccgccggggcagcctcgtctagcgcgcgccgcgcagacgcccccggagtcgccagctaccgcagccctcgccgcccagtgcccttcggcctcggggcgggcgcctgcgtcggtctccgcgaagcgggaaagcgcggcggccgccgggattcgggcgccgcggcagctgctccggctgccggccggcggccccgcgctcgcccgccccgcttccgcccgctgtcctgctgcacgaacccttccaactctcctttcctcccccacccttgagttacccctctgtctttcctgctgttgcgcgggtgctcccacagcggagcggagattacagagccgccggg

SEQ ID NO: 26 - DNA, 199 bp, Homo sapiens
gcaatccggagtgtcagctctccatctgtctctgcctggcaggcgcacgcgcccagcaccctgcctccggcgatgccgccccagcccctctgatggccctcctctctgctgccactcattccagaacaggaggcatgagcccggaacgcgcttgcttttaggagacagccactttctgtgtggtacgctggattcaagg

SEQ ID NO: 27 - DNA, 150 bp, Homo sapiens
atatcaacctgtttcctcctcctccttctcctcctcctccgtgacctcctcctcctctttctcctgagaaacttcgccccagcggtgcggagcgccgctgcgcagccggggagggacgcaggcaggcggcgggcagcgggaggcggcagc

SEQ ID NO: 28 - DNA, 167 bp, Saccharomyces cerevisiae
agccaaaataatgataacgagaataatatcaagaataccttagaacaacatcgacaacaacaacaggcattttcggatatgagtcacgtggagtattccagaattacaaaattttttcaagaacaaccactggagggatatacccttttctctcacaggtctgcgcc

SEQ ID NO: 29 - DNA, 696 bp, Anemonia majano
atgtctctttcaaagcatggcatcacacaagaaatgccgacgaaataccatatgaaaggcagtgtcaatggccatgaattcgagatcgaaggtgtaggaactggacacccttacgaagggacacacatggccgaattagtgatcataaagcctgcgggaaaaccccttccattctcctttgacatactgtcaacagtcattcaatacggaaacagatgcttcactaagtaccctgcagacctgcctgactatttcaagcaagcatacccaggtggaatgtcatatgaaaggtcatttgtgtatcaggatggaggaattgctacagcgagctggaacgttggtctcgagggaaattgcttcatccacaaatccacctatcttggtgtaaactttcctgctgatggacccgtaatgacaaagaagacaattggctgggataaagcctttgaaaaaatgactgggttcaatgaggtgttaagaggtgatgtgactgagtttcttatgctcgaaggaggtggttaccattcatgccagtttcactccacttacaaaccagagaagccggtcgaactgcccccgaatcatgtcatagaacatcacattgtgaggaccgaccttggcaagactgcaaaaggcttcatggtcaagctggtacaacatgctgcggctcatgttaaccctttgaaggttcaataa

SEQ ID NO: 30 - protein, 231 aa, Anemonia majano
MSLSKHGITQEMPTKYHMKGSVNGHEFEIEGVGTGHPYEGTHMAELVIIKPAGKPLPFSFDILSTVIQYGNRCFTKYPADLPDYFKQAYPGGMSYERSFVYQDGGIATASWNVGLEGNCFIHKSTYLGVNFPADGPVMTKKTIGWDKAFEKMTGFNEVLRGDVTEFLMLEGGGYHSCQFHSTYKPEKPVELPPNHVIEHHIVRTDLGKTAKGFMVKLVQHAAAHVNPLKVQ

SEQ ID NO: 31 - DNA, 720 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 32 - protein, 239 aa, Aequorea victoria
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 33 - DNA, 719 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaactgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 34 - protein, 239 aa, Aequorea victoria
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 35 - DNA, 711 bp, Aequorea victoria
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 36 - protein, 236 aa, Aequorea victoria
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 37 - DNA, 948 bp, Aequorea victoria
atggcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccgtccatctggaattcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagag

SEQ ID NO: 38 - protein, 316 aa, Aequorea victoria
MAEGSVARQPDLLTCDDEPIHIPGAIQPHGLLLALAADMTIVAGSDNLPELTGLAIGALIGRSAADVFDSETHNRLTIALAEPGAAVGAPITVGFTMRKDAGFIGSWHRHDQLIFLELEPPQRDVAEPQAFFRRTNSAIRRLQAAETLESACAAAAQEVRKITGFDRVMIYRFASDFSGEVIAEDRCAEVESKLGLHYPASTVPAQARRLYTINPVRIIPDINYRPVPVTPDLNPVTGRPIDLSFAILRSVSPVHLEFMRNIGMHGTMSISILRGERLWGLIVCHHRTPYYVDLDGRQACELVAQVLAWQIGVMEE

SEQ ID NO: 39 - DNA, 714 bp, Aequorea victoria
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggccgtcgagggcggccctctccccttcgccttcgacatcctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattga

SEQ ID NO: 40 - protein, 237 aa, Aequorea victoria
MVSKGEELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHKLN

SEQ ID NO: 41 - DNA, 63 bp, Homo sapiens
aagctgaaccctcctgatgagagtggccccggctgcatgagctgcaagtgtgtgctctcctga

SEQ ID NO: 42 - protein, 20 aa, Homo sapiens
KLNPPDESGPGCMSCKCVLS

SEQ ID NO: 43 - DNA, 66 bp, Porcine Teschovirus-1
ggaagcggagctactaacttcagcctgctgaagcaggctggagacgtggaggagaaccctggacct

SEQ ID NO: 44 - protein, 22 aa, Porcine Teschovirus-1
GSGATNFSLLKQAGDVEENPGP

SEQ ID NO: 45 - DNA, 63 bp, Thoseaasigna virus 2A
ggaagcggagagggcagaggaagtctgctaacatgcggtgacgtcgaggagaatcctggacct

SEQ ID NO: 46 - protein, 21 aa, Thoseaasigna virus 2A
GSGEGRGSLLTCGDVEENPGP

SEQ ID NO: 47 - DNA, 69 bp, Equine rhinitis A virus
ggaagcggacagtgtactaattatgctctcttgaaattggctggagatgttgagagcaaccctggacct

SEQ ID NO: 48 - protein, 23 aa, Equine rhinitis A virus
GSGQCTNYALLKLAGDVESNPGP

SEQ ID NO: 49 - DNA, 75 bp, Foot and Mouth Disease Virus
ggaagcggagtgaaacagactttgaattttgaccttctcaagttggcgggagacgtggagtccaaccctggacct

SEQ ID NO: 50 - protein, 25 aa, Foot and Mouth Disease Virus
GSGVKQTLNFDLLKLAGDVESNPGP

SEQ ID NO: 51 - DNA, 3686 bp, Homo sapiens
cttcagatagattatatctggagtgaagaatcctgccacctatgtatctggcatagtattctgtgtagtgggatgagcagagaacaaaaacaaaataatccagtgagaaaagcccgtaaataaaccttcagaccagagatctattctctagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataaaaaaaaaaaaaaaaaaaaaaaaaaa

SEQ ID NO: 52 - protein, 352 aa, Homo sapiens [sequence omitted]

SEQ ID NO: 53 - DNA, 2207 bp, Homo sapiens
cacttcctccccagacaggggtagtgcgaggccgggcacagccttcctgtgtggttttaccgcccagagagcgtcatggacctggggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaaaaaaaaa

SEQ ID NO: 54 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 55 - DNA, 3050 bp, Homo sapiens
tttttttgcttctgccccagatctttcctggacagtgcgtctcagcagttcagatccgggggcccccagctgacagagggcgtggggggttaaggcattaacccctcccagcctcttcctgaagaaaccacccagccttggcgcggcgctgggtgacttcgcgtagcaggcagggaactggccgcggcgagcgggactggccattggagtgctccgctgcggagggaggggaccccgactcgagtaagtttgcgagagcactacgcagtcagtcgggggcagcagcaagatgcgaagcgagccgtacagatcccgggctctccgaacgcaacttcgccctgcttgagcgaggctgcggtttccgaggccctctccagccaaggaaaagctacacaaaaagcctggatcactcatcgaaccacccctgaagccagtgaaggctctctcgcctcgccctctagcgttcgtctggagtagcgccaccccggcttcctggggacacagggttggcaccatggggcccaccagcgtcccgctggtcaaggcccaccgcagctcggtctctgactacgtcaactatgatatcatcgtccggcattacaactacacgggaaagctgaatatcagcgcggacaaggagaacagcattaaactgacctcggtggtgttcattctcatctgctgctttatcatcctggagaacatctttgtcttgctgaccatttggaaaaccaagaaattccaccgacccatgtactattttattggcaatctggccctctcagacctgttggcaggagtagcctacacagctaacctgctcttgtctggggccaccacctacaagctcactcccgcccagtggtttctgcgggaagggagtatgtttgtggccctgtcagcctccgtgttcagtctcctcgccatcgccattgagcgctatatcacaatgctgaaaatgaaactccacaacgggagcaataacttccgcctcttcctgctaatcagcgcctgctgggtcatctccctcatcctgggtggcctgcctatcatgggctggaactgcatcagtgcgctgtccagctgctccaccgtgctgccgctctaccacaagcactatatcctcttctgcaccacggtcttcactctgcttctgctctccatcgtcattctgtactgcagaatctactccttggtcaggactcggagccgccgcctgacgttccgcaagaacatttccaaggccagccgcagctctgagaagtcgctggcgctgctcaagaccgtaattatcgtcctgagcgtcttcatcgcctgctgggcaccgctcttcatcctgctcctgctggatgtgggctgcaaggtgaagacctgtgacatcctcttcagagcggagtacttcctggtgttagctgtgctcaactccggcaccaaccccatcatttacactctgaccaacaaggagatgcgtcgggccttcatccggatcatgtcctgctgcaagtgcccgagcggagactctgctggcaaattcaagcgacccatcatcgccggcatggaattcagccgcagcaaatcggacaattcctcccacccccagaaagacgaaggggacaacccagagaccattatgtcttctggaaacgtcaactcttcttcctagaactggaagctgtccacccaccggaagcgctctttacttggtcgctggccaccccagtgtttggaaaaaaatctctgggcttcgactgctgccagggaggagctgctgcaagccagagggaggaagggggagaatacgaacagcctggtggtgtcgggtgttggtgggtagagttagttcctgtgaacaatgcactgggaagggtggagatcaggtcccggcctggaatatattttctacccccctggagctttgattttgcactgagccaaaggtctagcattgtcaagctcctaaagggttcatttggcccctcctcaaagactaatgtccccatgtgaaagcgtctctttgtctggagctttgaggagatgttttccttcactttagtttcaaacccaagtgagtgtgtgcacttctgcttctttagggatgccctgtacatcccacaccccaccctcccttcccttcatacccctcctcaacgttcttttactttatactttaactacctgagagttatcagagctggggttgtggaatgatcgatcatctatagcaaataggctatgttgagtacgtaggctgtgggaagatgaagatggtttggaggtgtaaaacaatgtccttcgctgaggccaaagtttccatgtaagcgggatccgttttttggaatttggttgaagtcactttgatttctttaaaaaacatcttttcaatgaaatgtgttaccatttcatatccattgaagccgaaatctgcataaggaagcccactttatctaaatgatattagccaggatccttggtgtcctaggagaaacagacaagcaaaacaaagtgaaaaccgaatggattaacttttgcaaaccaagggagatttcttagcaaatgagtctaacaaatatgacatctgtctttggcacttttgttgatgtttatttcagaatgttgtgtgattcatttcaagcaacaacatggttgtattttgttgtgttaaaagtacttttcttgatttttgaatgtatttgtttcagcagaagtcattttattggatttttctaacccgtgttaacaccattgaatgtgtatttcttaagaaaataccaccctcttgtgcccttaaaagcattactttaactggtagggaacgccagaaacttttcagtccagctattcattagatagtaattgaagatatgtataaatattacaaagaataaaaatatattactgtctctttagtatggttttcagtgcaattaaaccgagagatgtcttgtttttttaaaaagaatagtatttaataggtttctgacttttgtggatcattttgcacatagctttatcaacttttaaacattaataaactgatttttttaaagatc

SEQ ID NO: 56 - protein, 382 aa, Homo sapiens
MGPTSVPLVKAHRSSVSDYVNYDIIVRHYNYTGKLNISADKENSIKLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNNFRLFLLISACWVISLILGGLPIMGWNCISALSSCSTVLPLYHKHYILFCTTVFTLLLLSIVILYCRIYSLVRTRSRRLTFRKNISKASRSSEKSLALLKTVIIVLSVFIACWAPLFILLLLDVGCKVKTCDILFRAEYFLVLAVLNSGTNPIIYTLTNKEMRRAFIRIMSCCKCPSGDSAGKFKRPIIAGMEFSRSKSDNSSHPQKDEGDNPETIMSSGNVNSSS

SEQ ID NO: 57 - DNA, 3589 bp, Homo sapiens
cggccgccctggggacgcagacgccaaggcccctccggccagggccgggagccgggccggcctagccagttctgaaagccccatggccccagcaggcctctgagccccaccatgggcagcttgtactcggagtacctgaaccccaacaaggtccaggaacactataattataccaaggagacgctggaaacgcaggagacgacctcccgccaggtggcctcggccttcatcgtcatcctctgttgcgccattgtggtggaaaaccttctggtgctcattgcggtggcccgaaacagcaagttccactcggcaatgtacctgtttctgggcaacctggccgcctccgatctactggcaggcgtggccttcgtagccaataccttgctctctggctctgtcacgctgaggctgacgcctgtgcagtggtttgcccgggagggctctgccttcatcacgctctcggcctctgtcttcagcctcctggccatcgccattgagcgccacgtggccattgccaaggtcaagctgtatggcagcgacaagagctgccgcatgcttctgctcatcggggcctcgtggctcatctcgctggtcctcggtggcctgcccatccttggctggaactgcctgggccacctcgaggcctgctccactgtcctgcctctctacgccaagcattatgtgctgtgcgtggtgaccatcttctccatcatcctgttggccatcgtggccctgtacgtgcgcatctactgcgtggtccgctcaagccacgctgacatggccgccccgcagacgctagccctgctcaagacggtcaccatcgtgctaggcgtctttatcgtctgctggctgcccgccttcagcatcctccttctggactatgcctgtcccgtccactcctgcccgatcctctacaaagcccactactttttcgccgtctccaccctgaattccctgctcaaccccgtcatctacacgtggcgcagccgggacctgcggcgggaggtgcttcggccgctgcagtgctggaggccgggggtgggggtgcaaggacggaggcggggcgggaccccgggccaccacctcctgccactccgcagctccagctccctggagaggggcatgcacatgcccacgtcacccacgtttctggagggcaacacggtggtctgagggtgggggtggaccaacaaccaggccagggcagaggggttcatggagaggccactgggtgaccccagatagagacttggggctactgagccagatgcccccgccccacagacctgggtgatgttgcaaatatttcacacctggaaaggccagataaggcactgactagtcacatagcagtgttgcagtgcggtcctgagggccagtccagtggctagtgtgacccctttagaactggatcctggggaggccagggcaggggacctgtgaagagccagggtgagggcaggcagcatttaaggggagctcagggcaggagcactttaccacctggtacaaaggatttttttttttttttgagacggaatcttgcactgctgcccaggctggagtgcagtggcgtgatctcggctcaccgcaagctccgcctcctgggttcatgtcgttctcctgcctcagcctcccaagtagctgggactataggcgcctgccaccacacctggctaattttttgtacctttagtagagatggggtttcaccgtgttagccaggatggtcttgatctcctgacctcgtgatccgcccgcctcggcctcccaaagtgctgggattacaggcgtgagccaccgtgcccggctttttttttttttttttttttttttttttttttttttgagatgaagtctcgctctgttgcccaggctggagagtgcagtggtacggtctcagctcactgcaacctccacctcccaggttcaagcgattctccagcctgagcctcctgagtagctgggattacaggtgcctaccaccacgcccaggtaattttttttttttttgtatttttagtagagacggggtttcaccatgttggccaggctggtctcgaactcctgacctcatgatccgcccgtgttggcctcccaaagtgtgggattacaggcgtaagccacctcacctggcggtacaaagaatttctgcattttcttccctggcccctagtcctgcaccgatttctccttttcgaatgtattcctcctgccaccttctctgggcaacttcgtgcgactacagaaccactgtcctgaggagctagaggcctcctctctgaccatccagagcccaaatccacagcttccccaaatttcatcagctgccacttgacgacttctccccgtctctctgaggcccggaaaccacggctggaggtggggaggggatggcggctgaggtccattcctcattctcagacctcattgctcagttgcactatttggggcacagaataatcaccaaaagtgagaaaaacgagtttgggtggctggggaggactttgggactcttgatgcaaggcgcaacttgagaaaattctgggtgtgatatttgcacagacaccctcctttcaaaaacagccaccccccaagctattctcagctccacacctgcagccccagctaaggtaccaggtctcctgagcaaggcagagagaagccttgagccttctctgtgtcttctttcaagaaccccgctgtgtcttctttcaagattttttttttgagacagtttcaagatttttgttttgtttttgagatggagtctcactgtgtcacccaggctgaggtggcagtggttcaatctccgttcactgccacctccacctcccgggttcaagcgattctcctgcttcagcctctcgagtagctgggactacaggcacctgccaccatgtctggctaatttttgtatttttagtagagacagggtttcactacgttggccaggctggtctcaaactcctgacctcaagtgatccgcccgcctcggcctccccaattgctgggattacaggcgtgagccactgtgcccggccttcttctttcaagttatatagaatggagcatgggggtggcagtggctagggacatttcctggggacactctcccctaaccccccagaaggacttcacaaaaacctgtggataatggaagggatgttacggtacaaacgtatatttatgtgtgtgtgtgtgtatgtgtgtgcgcgcgcgcgtgtgcacataggcgtgatgtctgtgaccctcctctcctcgtcacatttcccccagaatgaatgctgtcctgtctgctcatgtttgtgttgaagctgccaaagtcggggagctctggtcctgcccagacccctttggaattgctggcccatcctcccactggagagctggggtgcagctcaccttggggaaggaaacctcatgcctcagagtaatttcttgtgaatgcaaagcctgggggagcgggtctttggggggcaaggagccagtcaggggcttgtttcccctcatagagctccccagacgtgcctccgcaatgcctgaaacccagacctaggctaataaacggttcaatttctgttaaaaa

SEQ ID NO: 58 - protein, 353 aa, Homo sapiens
MGSLYSEYLNPNKVQEHYNYTKETLETQETTSRQVASAFIVILCCAIVVENLLVLIAVARNSKFHSAMYLFLGNLAASDLLAGVAFVANTLLSGSVTLRLTPVQWFAREGSAFITLSASVFSLLAIAIERHVAIAKVKLYGSDKSCRMLLLIGASWLISLVLGGLPILGWNCLGHLEACSTVLPLYAKHYVLCVVTIFSIILLAIVALYVRIYCVVRSSHADMAAPQTLALLKTVTIVLGVFIVCWLPAFSILLLDYACPVHSCPILYKAHYFFAVSTLNSLLNPVIYTWRSRDLRREVLRPLQCWRPGVGVQGRRRGGTPGHHLLPLRSSSSLERGMHMPTSPTFLEGNTVV

SEQ ID NO: 59 - DNA, 54 bp, Homo sapiens
atgggctgcatcaagagcaagcgcaaggacaacctgaacgacgacgaggccgcc

SEQ ID NO: 60 - DNA, 672 bp, Homo sapiens
ttagaattgatggcctcaccgttgacccgctttctgtcgctgaacctgctgctgctgggtgagtcgattatcctggggagtggagaagctaagccacaggcacccgaactccgaatctttccaaagaaaatggacgccgaacttggtcagaaggtggacctggtatgtgaagtgttggggtccgtttcgcaaggatgctcttggctcttccagaactccagctccaaactcccccagcccaccttcgttgtctatatggcttcatcccacaacaagataacgtgggacgagaagctgaattcgtcgaaactgttttctgccatgagggacacgaataataagtacgttctcaccctgaacaagttcagcaaggaaaacgaaggctactatttctgctcagtcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccactaa

SEQ ID NO: 61 - DNA, 24 bp, Artificial Sequence
  misc_feature: Synthetic oligonucleotide
tgagcataataaccataaatacta

SEQ ID NO: 62 - DNA, 24 bp, Artificial Sequence
  misc_feature: Synthetic oligonucleotide
tctgtgggactgtgttgactgtgg

SEQ ID NO: 63 - DNA, 20 bp, Artificial Sequence
  misc_feature: Synthetic oligonucleotide
agcaaatgtcctaaatgaat

SEQ ID NO: 64 - DNA, 24 bp, Artificial Sequence
  misc_feature: Synthetic oligonucleotide
tctgtgggactgtgttgactgtgg

SEQ ID NO: 65 - DNA, 24 bp, Artificial Sequence
  misc_feature: Synthetic oligonucleotide
tgagcataataaccataaatacta

SEQ ID NO: 66 - DNA, 24 bp, Artificial Sequence
  misc_feature: Synthetic oligonucleotide
gaggccaatgatgaagaggaagat

SEQ ID NO: 67 - DNA, 20 bp, Artificial Sequence
  misc_feature: Synthetic oligonucleotide
agcaaatgtcctaaatgaat

SEQ ID NO: 68 - DNA, 20 bp, Artificial Sequence
  misc_feature: Synthetic oligonucleotide
ctctggctgaaggtgctgtg

SEQ ID NO: 69 - DNA, 24 bp, Artificial Sequence
  misc_feature: Synthetic oligonucleotide
atcactttgagatccatgtttgca

SEQ ID NO: 70 - DNA, 25 bp, Artificial Sequence
  misc_feature: Synthetic oligonucleotide
aatccagggtttcgttctcatgcgc

SEQ ID NO: 71 - DNA, 25 bp, Artificial Sequence
  misc_feature: Synthetic oligonucleotide
agctgaagtgatcctgtctgcgctt

SEQ ID NO: 72 - DNA, 49 bp, Artificial Sequence
  misc_feature: Synthetic oligonucleotide
ccggagatctaggatcacttcagctcatctgcgcatgagaacgaaaccc

SEQ ID NO: 73 - DNA, 41 bp, Artificial Sequence
  misc_feature: Synthetic oligonucleotide
ccttgctcaccatgctaccagtcacagtcggagggttgttc

SEQ ID NO: 74 - DNA, 41 bp, Artificial Sequence
  misc_feature: Synthetic oligonucleotide
ccctccgactgtgactggtagcatggtgagcaagggcgagg

SEQ ID NO: 75 - DNA, 33 bp, Artificial Sequence
  misc_feature: Synthetic oligonucleotide
ccggctcgagctacttgtacagctcgtccatgc

(19) United States
(12) Patent Application Publication
(10) Pub. No.: US 2015/0065376 A1
(43) Pub. Date: Mar. 5, 2015

(54) EXPRESSION CONSTRUCTS ENCODING G PROTEIN COUPLED RECEPTORS AND METHODS OF USE THEREOF

(71) Applicants: Holger Knaut, New York, NY (US); Stephen Lewellis, New York, NY (US); Gayatri Venkiteswaran, Carteret, NJ (US)

(72) Inventors: Holger Knaut, New York, NY (US); Stephen Lewellis, New York, NY (US); Gayatri Venkiteswaran, Carteret, NJ (US)

(21) Appl. No.: 14/457,406

(22) Filed: Aug. 12, 2014

Related U.S. Application Data
(60) Provisional application No. 61/864,869, filed on Aug. 12, 2013.

(51) Int. Cl. G01N 33/50; C07K 14/705

(52) U.S. Cl. CPC G01N 33/5041; C07K 14/705; G01N 2333/705; G01N 2500/04; G01N 2500/10
USPC 506/9; 435/320.1

(57) ABSTRACT

Expression vectors comprising a first nucleic acid sequence encoding a G protein coupled receptor (GPCR), wherein the GPCR encoded thereby is expressed as a fusion protein with a first detectable marker/signal, and a second nucleic acid sequence encoding a second polypeptide that is or comprises a second detectable marker/signal, wherein the second polypeptide is expressed as a fusion protein with a membrane localizing sequence are encompassed herein. The first nucleic acid sequence encoding the GPCR and the second nucleic acid sequence encoding the second polypeptide are under the transcriptional control of the same promoter and are operably linked via, for example, an internal ribosomal entry site (IRES). The first and second detectable markers, moreover, emit distinct detectable signals. Cells comprising these expression vectors are also encompassed herein, as are methods of using same to screen for G protein coupled receptor modulators.

### CROSS REFERENCE TO RELATED APPLICATIONS

This application claims priority under 35 USC §119(e) from U.S. Provisional Application Ser. No. 61/864,869, filed Aug. 12, 2013, which application is herein specifically incorporated by reference in its entirety.

### GOVERNMENTAL SUPPORT

The research leading to the present invention was funded in part by Grant No. R01NS069839 awarded by the National Institutes of Health. The United States government has certain rights in the invention.

### FIELD OF THE INVENTION

The present invention pertains to the fields of molecular biology, G protein coupled receptors (GPCRs), methods for screening to identify GPCR ligands, and methods and uses for GPCR ligands identified thereby for therapeutic purposes. More specifically, the invention relates to constructs comprising a nucleic acid sequence encoding a GPCR, wherein the GPCR encoded thereby is expressed as a fusion protein with a first detectable marker/signal, and a nucleic acid sequence encoding a second polypeptide that is or comprises a second detectable marker/signal, wherein the second polypeptide is expressed as a fusion protein with a membrane localizing sequence. As described herein, the nucleic acid sequence encoding the GPCR and the nucleic acid sequence encoding the second polypeptide are under the transcriptional control of the same promoter and may be operably linked via an internal ribosomal entry site (IRES) and are thus, co-transcribed from the same promoter. The first and second detectable markers/signals, moreover, emit distinct detectable signals.

### BACKGROUND OF THE INVENTION

Several publications and patent documents are referenced in this application in order to more fully describe the state of the art to which this invention pertains. The disclosure of each of these publications and documents is incorporated by reference herein.

During animal development, homeostasis and disease, cells must move from one location to another to form tissues, assemble into organs, chase a pathogen or, in the case of cancer, populate sites of metastasis. Depending on the process, cells migrate as single cells, chains of cells or as tissue-like collectives of a few to hundreds of cells. In order to move in the correct direction, migrating cells need guidance cues. Studies over the last few decades have revealed the identity of many guidance cues. These guidance cues are often secreted from the target tissue and form an attractant gradient, from which migrating cells derive directional information (Parent, 1999; Rørth, 2011; Swaney et al., 2010). Migrating cells can be guided by long-range attractant gradients emanating from a source at the target tissue (for example (Montell, 2003)), shifting expression domains of the attractant (for example (Affolter and Caussinus, 2008)) or the graded distribution of an immobilized attractant (for example (Weber et al., 2013)). In the simplest model, attractants are secreted from a local source and degraded by a local sink, generating a linear gradient at steady-state. Francis Crick showed in 1970 that this source-sink model can generate stable, linear gradients over several hundreds of μm (Crick, 1970).

The classic example for single cell migration is the slime mold Dictyostelium (reviewed in (Parent, 1999)). Dictyostelium cells are attracted by cyclic adenosine monophosphate (cAMP) and move towards higher cAMP concentrations. The cells are about 10 μm in diameter and sense differences in cAMP concentration of as low as 1% across themselves, but migrate most efficiently when this difference is 3% (Fisher et al., 1989). Intriguingly, Dictyostelium migrate towards higher cAMP concentrations within pre-steady-state gradients with temporally increasing cAMP concentration as well as stable, steady-state gradients (Fisher et al., 1989). It is thought that Dictyostelium achieves sensitivity to concentration differences of cAMP and robustness to fluctuations in cAMP concentration by integrating and reinforcing information about local cAMP concentrations sensed by the cAMP receptors on the cell surface (Cai and Devreotes, 2011).

GPCRs comprise a super family of seven transmembrane-spanning proteins that are activated by a diverse array of extracellular ligands, including biogenic amines, amino acids, ions, small and large peptides, and bioactive lipids. GPCRs are expressed in virtually all tissues and signaling through these receptors is known to contribute to a multiplicity of cellular and physiological processes, including chemotaxis, inflammation, neurotransmission, cell proliferation, cardiac and smooth muscle contractility, and visual and chemosensory perception. See, for example, Cacace et al. Drug Discovery Today 8:785-792, 2003; Bockaert et al. Int Rev Cytol 212:63-132, 2002; Pierce et al. Nat Rev Mol Cell Biol 3:639-650, 2002.

### SUMMARY OF INVENTION

In a first aspect, a synthetic expression vector or cassette comprising a single transcriptional unit is described herein, wherein the single transcriptional unit comprises a first nucleic acid sequence encoding a G protein coupled receptor operably linked to a first detectable marker and a second nucleic acid sequence encoding a second detectable marker operably linked to a membrane localizing domain, wherein the first nucleic acid sequence is operably linked to the second nucleic acid sequence via an internal ribosomal entry site (IRES) or nucleic acid sequences encoding a viral 2A peptide sequence and the first and second detectable markers emit distinct detectable signals.

In embodiments wherein the expression vector or cassette comprises an IRES, bicistronic transgenes facilitate the simultaneous expression of the GPCR fusion protein (GPCR-first detectable marker) and the membrane localized second detectable marker. In embodiments wherein the expression vector or cassette comprises nucleic acid sequences encoding a viral 2A peptide sequence, the viral 2A peptide sequence facilitates multiprotein expression from a single open reading frame (ORF). In either case, operably linking the first and second nucleic acid sequences via an IRES or nucleic acid sequences encoding a viral 2A peptide sequence confers stoichiometric expression of the GPCR fusion protein and the membrane localized second detectable marker. The structural features of the expression vector or cassette described herein, therefore, confer the functional property of stoichiometric expression of polypeptides encoded thereby, which in turn makes these expression vectors or cassettes well suited for use as GPCR signaling sensors. Absent stoichiometric expression of the encoded polypeptides, the engagement of a GPCR by ligand and subsequent internalization does not allow for quantitative measurements and detection of small changes, especially in cases where the GPCR is localized both to the membrane and to the inside of the cell.

In an aspect thereof, an expression vector comprising a single transcriptional unit is envisioned, wherein the single transcriptional unit comprises two independent open reading frames, wherein a first open reading frame comprises a first nucleic acid sequence encoding a G protein coupled receptor operably linked to a first detectable marker and a second open reading frame comprises a second nucleic acid sequence encoding a second detectable marker operably linked to a membrane localizing domain, wherein the first open reading frame is operably linked to the second open reading frame via an internal ribosomal entry site (IRES) and the first and second detectable markers emit distinct detectable signals.

In a particular embodiment, the G protein coupled receptor is CXCR1 (MIM 146929), CXCR2 (MIM 146928), CXCR3 (MIM 300574), CXCR4 (MIM 162643), CXCR5 (MIM 601613), CXCR6 (MIM 605163), CXCR7 (MIM 610376), CCR1 (MIM 601159), CCR2 (MIM 601267), CCR3 (MIM 601268), CCR4 (MIM 604836; See also SEQ ID NOs: 1-4 of FIG. 8), CCR5 (MIM 601373; SEQ ID NOs: 51-52), CCR6 (MIM 601835), CCR7 (MIM 600242; SEQ ID NOs: 53-54), CCR8 (MIM 601834), CCR9 (MIM 604738), CCR10 (MIM 600240), CX3CR1 (MIM 601470), GPR15 (MIM 601166; See also SEQ ID NOs: 5-8 of FIGS. 9 and 10), FPR (MIM 136537), D6 (MIM 602648), DARC/Duffy (MIM 613665), CCX-CKR (MIM 606065), PAR1 (MIM 187930), PAR2 (MIM 600933), PAR3 (MIM 601919), PAR4 (MIM 602779), ADRB1 (MIM 109630), ADRB2 (MIM 109690), ADRB3 (MIM 109691), ADRA1B (MIM 104220), ADRA1D (MIM 104219), ADRA1A (MIM 104221), ADRA2A (MIM 104210), S1PR1 (MIM 601974; SEQ ID NO: 55-56), S1PR2 (MIM 605111; SEQ ID NO: 57-58), S1PR3 (MIM 601965), S1PR4 (MIM 603751) or S1PR5 (MIM 605146). All of the aforementioned sequences are available to the public and accessible via the Online Catalog of Human Genes and Genetic Disorders of the worldwide web, which comprises a plurality of Mendelian Inheritance in Man (MIM) entries corresponding to human genes. The entire content of each of the aforementioned MIM entries is incorporated herein by reference.

In another particular embodiment, wherein the first and second nucleic acid sequences are operably linked via an IRES, the IRES may be a viral IRES or a cellular IRES. In a more particular embodiment, the viral IRES is the EMCV-R IRES, CrPV IGR IRES, HCV type 1a IRES, FMDV type C IRES, HAV HM175 IRES, PV type 1 Mahoney IRES, PV type 3 Leon IRES, AEV IRES, CSFV IRES, or ERAV 245-961 IRES. In yet another particular embodiment, the cellular IRES is the APAF-1 IRES, BCL2 IRES, ELH IRES, FMR1 IRES, HSP70, Kv1.4 1.2 IRES, LEF1 IRES, MTG8a IRES, MYB IRES, or URE2 IRES.

It is to be understood that any transmembrane domain can be used to tether the second detectable marker to the membrane. Exemplary membrane localizing domains include the CaaX motif, the transmembrane domain of the receptor tyrosine kinase lyn, and the transmembrane domain of CD8 protein.

In an embodiment, the expression vector comprises a single promoter that drives expression of the first and second nucleic acid sequences.

In a particular embodiment, each of the first and second detectable markers is a fluorescent marker. In a more particular embodiment, the first detectable marker is red fluorescent protein. In another particular embodiment, the second detectable marker is green fluorescent protein.

In another aspect, a method for screening to identify a modulator of a G protein coupled receptor is presented, the method comprising providing a plurality of cells comprising an expression vector or cassette described herein; contacting the plurality of cells with at least one candidate compound or agent; and measuring detectable signals of the first and second detectable markers in the presence or absence of the at least one candidate compound or agent to determine if the presence of the at least one candidate compound or agent causes a change in the detectable signals of the first and second detectable markers, wherein a change in detectable signals in the presence of the at least one candidate compound or agent identifies the at least one candidate compound or agent as a G protein coupled receptor modulator.

In an embodiment of the method, the change in detectable signals reflects relocalization of the first detectable signal, wherein the relocalization is detected by a reduction in co-localization of detectable signals of the first and second detectable markers. In a further embodiment, the first detectable signal relocalizes from the plasma membrane to early endosomes or endosomal vesicles of the plurality of cells.

In a particular embodiment of the method, the first and second detectable markers are fluorescent markers. In such an embodiment, the change in detectable signals reflects relocalization of the first detectable signal and a reduction in colocalization of immunofluorescence signals of the first and second detectable markers in the presence of the at least one candidate compound or agent identifies the at least one candidate compound or agent as a G protein coupled receptor modulator. In a more particular embodiment, the first detectable marker is red fluorescent protein. In yet another particular embodiment, the second detectable marker is green fluorescent protein.

The method may further comprise incubation of the cells under conditions that stimulate G protein coupled receptor activity, such as, for example, the addition of a known ligand or agonist of the G protein coupled receptor that results in an increase in G protein coupled receptor activity. Such conditions facilitate screening to identify an antagonist of the G protein coupled receptor. Under activated conditions, the presence of a GPCR antagonist would be reflected in an increase in co-localization of the two distinct detectable markers/signals.

In an embodiment of the method, the at least one candidate compound or agent is a component of a library of compounds or agents or a component of a tissue extract. Such libraries may comprise small molecules, small peptides, large peptides, biogenic amines, amino acids, ions, or bioactive lipids. Small chemical and small molecule libraries, as well as RNAi libraries are envisioned herein for use in screening methods. Various chemical agents predicted to be potential modulators based on biological activity studies are also encompassed herein for use in screening methods.

In a particular embodiment of the method, the method is a computer-assisted method.

In another aspect, a method for screening to identify a modulator of a G protein coupled receptor is described, the method comprising: providing a plurality of cells comprising an expression vector or cassette comprising a first and a second nucleic acid sequence, wherein the first nucleic acid sequence encodes a G protein coupled receptor operably linked to a first detectable marker and the second nucleic acid sequence encodes a second detectable marker operably linked to a membrane localizing domain, wherein the first nucleic acid sequence is operably linked to the second nucleic acid sequence via an internal ribosomal entry site (IRES) or a peptide 2A sequence and the first and second detectable markers emit distinct detectable signals; contacting the plurality of cells with at least one candidate compound or agent; and measuring detectable signals from the first and second detectable markers in the presence or absence of the at least one candidate compound or agent to determine if the presence of the at least one candidate compound or agent causes a change in the detectable signals from the first and second detectable markers, wherein a change in detectable signals in the presence of the at least one candidate compound or agent identifies the at least one candidate compound or agent as a G protein coupled receptor modulator.

In a particular embodiment of the method, the method is a computer-assisted method.

In an embodiment of the method, the G protein coupled receptor is CXCR1 (MIM 146929), CXCR2 (MIM 146928), CXCR3 (MIM 300574), CXCR4 (MIM 162643), CXCR5 (MIM 601613), CXCR6 (MIM 605163), CXCR7 (MIM 610376), CCR1 (MIM 601159), CCR2 (MIM 601267), CCR3 (MIM 601268), CCR4 (MIM 604836; See also SEQ ID NOs: 1-4 of FIG. 8), CCR5 (MIM 601373; SEQ ID NOs: 51-52), CCR6 (MIM 601835), CCR7 (MIM 600242; SEQ ID NOs: 53-54), CCR8 (MIM 601834), CCR9 (MIM 604738), CCR10 (MIM 600240), CX3CR1 (MIM 601470), GPR15 (MIM 601166; See also SEQ ID NOs: 5-8 of FIGS. 9 and 10), FPR (MIM 136537), D6 (MIM 602648), DARC/Duffy (MIM 613665), CCX-CKR (MIM 606065), PAR1 (MIM 187930), PAR2 (MIM 600933), PAR3 (MIM 601919), PAR4 (MIM 602779), ADRB1 (MIM 109630), ADRB2 (MIM 109690), ADRB3 (MIM 109691), ADRA1B (MIM 104220), ADRA1D (MIM 104219), ADRA1A (MIM 104221), ADRA2A (MIM 104210), S1PR1 (MIM 601974; SEQ ID NO: 55-56), S1PR2 (MIM 605111; SEQ ID NO: 57-58), S1PR3 (MIM 601965), S1PR4 (MIM 603751) or S1PR5 (MIM 605146).

In a further embodiment of the method, wherein the first and second nucleic acid sequences are operably linked via an IRES, the IRES may be a viral IRES or a cellular IRES. In a more particular embodiment, the viral IRES is the EMCV-R IRES, CrPV IGR IRES, HCV type 1a IRES, FMDV type C IRES, HAV HM175 IRES, PV type 1 Mahoney IRES, PV type 3 Leon IRES, AEV IRES, CSFV IRES, or ERAV 245-961 IRES. In yet another embodiment, the cellular IRES is the APAF-1 IRES, BCL2 IRES, ELH IRES, FMR1 IRES, HSP70, Kv1.4 1.2 IRES, LEF1 IRES, MTG8a IRES, MYB IRES, or URE2 IRES.

As understood in the art, essentially any transmembrane domain can be used to tether the second detectable marker to the membrane. Exemplary membrane localizing domains include the CaaX motif, the transmembrane domain of the receptor tyrosine kinase lyn, and the transmembrane domain of CD8 protein.

In an embodiment of the method, the change in detectable signals reflects relocalization of the first detectable signal, wherein the relocalization is detected by a reduction in co-localization of detectable signals of the first and second detectable markers. In a particular embodiment thereof, the first detectable signal relocalizes from plasma membranes to early endosomes or endosomal vesicles of the plurality of cells. Under circumstances wherein the screening method detects a reduction of or decrease in co-localization of the first and second detectable markers in the presence of a candidate agent or molecule, the candidate agent or molecule is identified as potential ligand or agonist of the GPCR.

In a particular embodiment of the method, the first and second detectable markers are fluorescent markers. Accordingly, a change in detectable immunofluorescent signals reflects relocalization of the first detectable signal and a reduction in colocalization of immunofluorescent signals of the first and second detectable markers in the presence of the at least one candidate compound or agent identifies the at least one candidate compound or agent as a G protein coupled receptor modulator (e.g., a ligand or agonist).

In an embodiment of the method, the expression vector comprises a single promoter that drives expression of the first and second nucleic acid sequences.

In another embodiment of the method, the first detectable marker is red fluorescent protein. In a further embodiment thereof, the second detectable marker is green fluorescent protein.

In a particular embodiment, the method further comprises incubation of the cells under conditions that stimulate G protein coupled receptor activity, such as, for example, the addition of a known ligand or agonist of the G protein coupled receptor that results in an increase in G protein coupled receptor activity. Such conditions facilitate screening to identify an antagonist of the G protein coupled receptor. Under activated conditions, the presence of a GPCR antagonist would be reflected in an increase in co-localization of the two distinct detectable markers/signals.

In another embodiment of the method, the at least one candidate compound or agent is a component of a library of compounds or agents or a component of a tissue extract. Such libraries may comprise small molecules, small peptides, large peptides, biogenic amines, amino acids, ions, or bioactive lipids.

As indicated herein with regard to expression vectors or cassettes, it is to be understood that any transmembrane domain can be used to tether the second detectable marker to the membrane in methods described herein. Exemplary membrane localizing domains include the CaaX motif, the transmembrane domain of the receptor tyrosine kinase lyn, and the transmembrane domain of CD8 protein.

In a particular embodiment, expression vectors or cassettes described herein or used in methods described herein comprise the CaaX motif sequence, which serves as the membrane localization sequence that tethers the second detectable marker to the membrane. Cells comprising such vectors and methods using same are also encompassed herein. Nucleic (SEQ ID NO: 41) and amino acid (SEQ ID NO: 42) sequences for the CaaX motif are presented below:

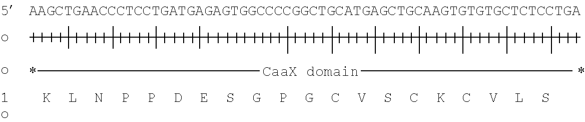

In alternative embodiments, the membrane localization sequence that tethers the second detectable marker to the membrane is the transmembrane domain from the receptor tyrosine kinase lyn or the transmembrane protein CD8. The nucleotide sequence of the lyn transmembrane domain is:

(SEQ ID NO: 59)
ATGGGCTGCATCAAGAGCAAGCGCAAGGACAACCTGAACGACG
ACGAGGCCGCC.

The nucleotide sequence of the transmembrane protein CD8 is as follows:

(SEQ ID NO: 60)
TTAGAATTGATGGCCTCACCGTTGACCCGCTTTCTGTCGCTGA
ACCTGCTGCTGCTGGGTGAGTCGATTATCCTGGGGAGTGGAGA
AGCTAAGCCACAGGCACCCGAACTCCGAATCTTTCCAAAGAAA
ATGGACGCCGAACTTGGTCAGAAGGTGGACCTGGTATGTGAAG
TGTTGGGGTCCGTTTCGCAAGGATGCTCTTGGCTCTTCCAGAA
CTCCAGCTCCAAACTCCCCCAGCCCACCTTCGTTGTCTATATG
GCTTCATCCCACAACAAGATAACGTGGGACGAGAAGCTGAATT
CGTCGAAACTGTTTTCTGCCATGAGGGACACGAATAATAAGTA
CGTTCTCACCCTGAACAAGTTCAGCAAGGAAAACGAAGGCTAC
TATTTCTGCTCAGTCATCAGCAACTCGGTGATGTACTTCAGTT
CTGTCGTGCCAGTCCTTCAGAAAGTGAACTCTACTACTACCAA
GCCAGTGCTGCGAACTCCCTCACCTGTGCACCCTACCGGGACA
TCTCAGCCCCAGAGACCAGAAGATTGTCGGCCCCGTGGCTCAG
TGAAGGGGACCGGATTGGACTTCGCCTGTGATATTTACATCTG
GGCACCCTTGGCCGGAATCTGCGTGGCCCTTCTGCTGTCCTTG
ATCATCACTCTCATCTGCTACCACTAA.

Other sequences and/or motifs that may be used to tether proteins to the membrane are described in, for example, Zlatkine et al. (1997, J Cell Science 110:673-679), which describes S-acylation.

In another embodiment, the expression vector or cassette described herein comprises nucleic acid sequences encoding a viral 2A peptide sequence which serves to operably link the first nucleic acid sequence to the second nucleic acid sequence. The nucleic acid sequences for P2A, T2A, E2A, and F2A listed below are designated SEQ ID NOs: 43, 45, 47, and 49, respectively. In a more particular embodiment, examples for the viral 2A peptide sequences are as follows:

(P2A; SEQ ID NO: 44)
GSGATNFSLLKQAGDVEENPGP,
(T2A; SEQ ID NO: 46)
GSGEGRGSLLTCGDVEENPGP,
(E2A; SEQ ID NO: 48)
GSGQCTNYALLKLAGDVESNPGP
and
(F2A; SEQ ID NO: 50)
GSGVKQTLNFDLLKLAGDVESNPGP.

| | |
|---|---|
| P2A | GGA AGC GGA GCT ACT AAC TTC AGC CTG CTG AAG CAG |
| | G S G A T N P S L L K Q |
| | GCT GGA GAC GTG GAG GAG AAC CCT GGA CCT |
| | A G D V E E N P G P |
| T2A | GGA ACC GGA GAG [symbol] GC AGA GGA AGT CTG CTA ACA TGC |
| | G S G E G R G S L L T C |
| | GCT GAC GTC GAG GAG AAT CCT GGA CCT |
| | G D V E E N P G P |
| E2A | GGA AGC GGA CAG TGT ACT AAT TAT GCT CTC TTG AAA |
| | G S G Q C T N Y A L L K |
| | TTG GCT GGA GAT GTT GAG AGC AAC CCT GGA CCT |
| | L A G D V E S N P G P |
| F2A | GGA AGC GGA GTG AAA CAG ACT TTG AAT TTT GAC CTT |
| | G S G V K Q T L N F D L |
| | CTC AAG TTG GCG GGA GAC GTG GAG TGC AAC CCT GGA |
| | L K L A G D V E S N P G |
| | CCT |
| | P |

[symbol]

indicates data missing or illegible when filed

See, for example, Kim et al. 2011, PLoS ONE 6(4): e18556. doi:10.1371/journal.pone.0018556; and Szymczak-Workman et al. Cold Spring Harbor Protoc; 2012; doi:10.1101/pdb.ip067876, the entire content of each of which is incorporated herein by reference, for reviews relating to 2A peptide sequences.

Also encompassed herein are methods directed to using expression vectors or cassettes described herein that comprise nucleic acid sequences encoding a viral 2A peptide sequence.

Other features and advantages of the invention will be apparent from the following description of the preferred embodiments thereof, and from the claims.

### BRIEF DESCRIPTION OF THE DRAWINGS

FIG. 1A-D. Expression and requirement of Sdf1a and its receptors Cxcr4b, Cxcr7a and Cxcr7b during primordium migration. (A) Live images of embryos of the indicated stage and genotype expressing GFP from the cldnB promoter in the migrating primordium (arrow) and deposited neuromasts (arrowheads). (B) Fluorescent in situ hybridization against cxcr4b, cxcr7a or cxcr7b (middle row) and antibody staining against GFP (bottom row) in tg(cldnB:lynlynGFP) embryos at 36 19 hours post fertilization (hpf). Scale bar 50 μm. (C) In situ hybridization against sdf1a mRNA (red) labeling the stripe of chemokine expression along the migration route of the primordium (green) in a tg(cldnB:lynlynGFP) embryo at 36 hpf. Anterior is to the left and posterior is to the right. (D) Quantification of primordium migration at 48 hpf in wild-type, sdf1a−/− and cxcr7 deficient embryos. The vertical bars represent the average position of the primordium in somites, the error bars represent SD and the circles represent the positions of individual primordia. 48 hpf embryo schematic adapted from reference (Kimmel et al., 1995).

FIG. 2A-F. Cxcr7 is required for Sdf1a-GFP sequestration by the primordium. (A, B) Average fluorescence intensity of Sdf1a-GFP protein along the stripe of chemokine producing cells underneath primordia of wild-type embryos (A) and tg(hsp70;cxcr7b) embryos (B) at 36 hpf normalized to the mean (A) or the mean of average intensities of equal numbers of heat-shocked wild-type control embryos (B, inset). (C) Single sections along the medial-lateral and anterior-posterior axis from 36 hpf embryos of indicated genotypes immunostained for Claudin-B and GFP. Scale bar corresponds to 10 μm. The inset represents the section bounded by the yellow box in the overlay. Arrowheads indicate Sdf1a-GFP puncta inside the primordium. (D) Distribution of intensities of Sdf1a-GFP puncta from 12 primordia of indicated genotypes. (E) Distribution of intensities of Sdf1a-GFP puncta from 12 primordia along the anterior-posterior axis. Each dot (D and E) represents an individual punctum (red indicates cxcr7b−/−; cxcr7a morphant primordia; black indicates wild-type primordia). In cxcr7 deficient embryos the Sdf1a-GFP puncta inside the primordia are shifted towards the posterior because the primordia are shorter and wider than in wild-type embryos. 0 μm represents the front of each primordium in A-E. (F) Fraction of Sdf1a-GFP found inside the primordium of the total Sdf1a-GFP (sum of Sdf1a-GFP inside the primordium and outside along chemokine expressing cells) in the indicated genotype. Each dot represents the fraction of Sdf1a-GFP in an individual primordium. Horizontal bars represent the mean+/−SEM. (See also FIG. S1)

FIG. 3A-E. A quantitative signaling reporter for Sdf1. (A, B) Schematic of Sdf1-signaling sensor construct (A) and concept (B). (C) Left: Mean FmemRed/FmemGreen ratio along the anterior-posterior axis of n≧20 primordia from embryos injected with the indicated amount of translation blocking morpholino against sdf1a. 0 μm represents the front of each primordium, circles are mean ratios and error bars represent SEM. Open circles indicate ratios greater than the mean FmemRed/FmemGreen ratio in Sdf1a−/− primordia. Right: Average shift of the FmemRed/FmemGreen ratio curves of the embryos injected with the indicated amount of morpholino. The error bars represent SD. (D) Single confocal slices through the primordium in live 36 hpf embryos of the indicated genotypes at the indicated stages, all carrying the Sdf1-signaling sensor. The FmemRed/FmemGreen images are inverted heat maps of the ratio. (E) Mean FmemRed/FmemGreen along the anterior-posterior axis of n≧10 primordia with 0 μm representing the front of each primordium. Red circles indicate the mean FmemRed/FmemGreen in embryos of the indicated genotype; black circles, where present, indicate the mean FmemRed/FmemGreen of wild-type embryos or heat-shocked control embryos. Grey bars indicate SEM. Anterior is to the left and dorsal is up in D. Scale bar is 20 μm.

FIG. 4A-B. Cxcr7 generates the Sdf1-signaling gradient across the primordium. (A) Single confocal slices through the primordium in live 36 hpf embryos of the indicated genotypes, all carrying the Sdf1-signaling sensor. The FmemRed/FmemGreen images are inverted heat maps of the ratio. (B) Mean FmemRed/FmemGreen along the anterior-posterior axis of n≧10 primordia with 0 μm representing the front of each primordium. Red circles indicate the mean FmemRed/FmemGreen in embryos of the indicated genotype; black circles, where present, indicate the mean FmemRed/FmemGreen of wild-type embryos or heat-shocked control embryos. Grey bars indicate SEM. Y-axes are scaled identical with the exception of tg(hsp70:cxcr7b). Anterior is to the left and dorsal is up in A. Scale bar is 20 μm.

FIG. 5A-C. Cxcr7 modifies the Sdf1-signaling gradient across the primordium at the tissue-level. (A) Single confocal slices through mosaic primordia in live 36 hpf embryos of the indicated genotypes. (B) Quantification of mean FmemRed/FmemGreen of host cells (black dots, grey bars SEM) and donor cells (red dots, light red bars SEM) across the anterior-posterior axis of primordia shown in A. (C) FmemRed/FmemGreen ratio on the host cells only across wild-type-wild-type and cxcr7 deficient-wild-type chimeric primordia containing the Sdf1-signaling sensor imaged at 36 hpf. The front of the primordium is at 0 μm. The grey bars indicate SEM. There is overlap between the 95% confidence interval for the slopes, P=0.06.

FIG. 6A-H. Kinetics of Sdf1-signaling gradient formation. (A-C) Mean FmemRed/FmemGreen along the anterior-posterior axis in tg(hsp70:sdf1a) (black dots, n=8) and tg(hsp70:sdf1a);cxcr7b−/− (red dots, n=2) at the indicated number of minutes after induction of a pulse of global Sdf1a expression imaged at the same time. The front of the primordium is at 0 μm. Grey bars indicate SEM. (D-F) Relationships of the slope of the gradient, speed of the primordium, and time (n=8). In D and E, solid black circles indicate mean, grey bars indicate SEM. In F, the grey line connects the data points in chronological order, as indicated by red arrows. (G, H) Sdf1-signaling gradient formation in vivo (G) and signaling competent Sdf1a protein gradient formation predicted in silico (H). 0 mins (post heat-shock) in H roughly corresponds to 360 mins in G.

FIG. 7A-F. Model for the evolution of chemokine gradient with different values of velocity (u) and effective diffusion coefficient (D). (A) Schematic of the geometry of the model, in which the primordium sits on top of a thin reservoir of chemokine of thickness L (typically <1 μm) formed above the stripe. Region of primordium absorbing chemokine (sink) with flux J₀ is at b>x>0, y=L where b=20 μm here. Cytokine is produced by a row of cells in the stripe below y=0 forming a source with flux J₁ at x>0, y=0. The gradient is sensed over the forward surface of the primordium which occupies a>x>b, y=L where a is typically 100 μm and there is no flux over this region. Chemokine is degraded throughout the reservoir with rate constant k. Two zones are defined for purposes of calculation: Zone A represents b>x₁>0 and Zone B represents a>x₂>b. In panels (B-E) the colored solid lines correspond to the gradients calculated with u=0 and the colored dashed lines are calculated with the specified u. Each gradient is calculated at the time indicated in the legend, assuming that C/C₀=1 at t=0. The dotted black line that coincides with the gradient for t=200 min calculated assuming steady-state conditions. The degradation rate constant is fixed at k=0.0003 s⁻¹. In each panel R=J₀/J₁ is the ratio of flux values chosen so that the steady-state baseline value of C/C₀ corresponds to the measured value of 0.14. (B) The value of u is the measured value and D is the free diffusion value (Veldkamp, 2005). The curves representing the effect of the moving primordium are indistinguishable from the curves when the primordium is stationary. (C) The value of u is set at 20× the measured value and D remains at D_{free}. The gradients generated by the moving primordium are steeper and reach a steady-state earlier than those from the stationary primordium. (D) The velocity is back to the measured value but D=D_{free}/4. The gradients are now steeper but the movement has negligible influence. (E) The velocity remains at the measured value but D=D_{free}/20. The gradients continue to become steeper and the movement has some influence on the curves. The gradients also begin to visibly depart from straight lines. The gradient at 200 min most resembles the experimental results and suggests that the effective value of D is less than D_{free}. (F) Model for how the migrating primordium generates a chemokine-signaling gradient across itself. In the pre-steady-state, sequestration of Sdf1a protein by Cxcr7 decreases Sdf1a protein selectively beneath the trailing half of the primordium resulting in reduced chemokine signaling in the rear. Diffusion from areas of higher Sdf1a protein concentration equilibrates the chemokine distribution across the primordium, resulting in a linear, stable signaling gradient.

FIG. 8A-D. Nucleic and amino acid sequences for CXCR4 variants. Nucleic (A) and amino (B) acid sequences of Homo sapiens chemokine (C-X-C motif) receptor 4 (CXCR4), transcript variant 2, mRNA; NCBI Reference Sequence: NM_{—}003467.2. Nucleic (C) and amino (D) acid sequences of Homo sapiens chemokine (C-X-C motif) receptor 4 (CXCR4), transcript variant 1, mRNA; NCBI Reference Sequence: NM_{—}001008540.1

FIG. 9A-D. Nucleic and amino acid sequences for human and mouse GPR15. Nucleic (A) and amino (B) acid sequences of Homo sapiens GPR15. Nucleic (C) and amino (D) acid sequences of mouse GPR15.

FIG. S1. Schematic overview of the sdf1a:sdf1a-GFP transgene.

FIG. S2. Schematic overview of the cxcr4b:cxcr4b-kate2-IRES-GFP-CaaX transgene.

FIG. S3A-D. The Sdf1-signaling sensor measurements are independent of the expression levels of the Sdf1-signaling sensor. (A) Derivation of equation for reversible binding of Sdf1 to Cxcr4b (equation 4) using the definition of the dissociation constant (equation 1) for the reversible reaction Cxcr4b_{eq}+Sdf1_{eq} □ Cxcr4b-Sdf1_{eq}, where Cxcr4b_{eq} is free receptor, Sdf1_{eq} is free ligand, and Cxcr4b-Sdf1_{eq} is receptor-ligand complex, and mass balance for Sdf1 (equation 2) and Cxcr4b (equation 3). (B) Graph of the fraction of free Cxcr4b versus free Sdf1 with a dissociation constant of 4 nM REF. (C) Mean FmemGreen/FmemRed ratio values across 100 μm beginning at the front of the primordium in 36 hpf wild type embryos with increasing levels of expression of the signaling sensor indicated in D. Note the two signaling sensor lines used have Cxcr4b fused to GFP followed by an IRES that drives expression of membrane tethered kate2 (tg(cxcr4b:cxcr4b-GFP-IRES-Kate2-Caax)). (D) Mean FmemRed intensity values across 100 μm beginning at the front of the primordium in 36 hpf embryos carrying different combinations and copy number of the signaling sensor transgenes (blue: tg(cxcr4b:cxcr4b-GFP-IRES-Kate2-Caax)p1/+, n=14, green: tg(cxcr4b:cxcr4b-GFP-IRES-Kate2-Caax)p7/+, n=17, yellow: tg(cxcr4b:cxcr4b-GFP-IRES-Kate2-Caax)p1/tg(cxcr4b:cxcr4b-GFP-IRES-Kate2-Caax)p7, n=10, red: tg(cxcr4b:cxcr4b-GFP-IRES-Kate2-Caax)p7/tg(cxcr4b:cxcr4b-GFP-IRES-Kate2-Caax)p7, n=16). In C and D the front of the primordium is at 0 μm. The colored and grey bars indicate SEM in C and D, respectively.

FIG. S4A-D. Recombinant human SDF1 activates the human version of the Sdf1-signaling sensor. (A and B) Flp-In™ T-REx™ cells expressing the human version of the Sdf1-signaling sensor from a tetracycline-inducible promoter (Invitrogen) before (A) and 40 min after (B) vehicle treatment. (C and D) Flp-In™ T-REx™ cells expressing the human version of the Sdf1-signaling sensor from a tetracycline-inducible promoter (Invitrogen) before (C) and 40 min after (D) addition of recombinant human SDF1. Arrows indicate internalized CXCR4-RFP.

### DETAILED DESCRIPTION OF THE INVENTION

GPCRs comprise the largest family of signaling receptors in humans (about 800 genes) and mediate many biological responses ranging from immune cell trafficking to neuronal communication. Therefore, GPCRs are common targets for drug screening and development. As detailed herein, expression vectors and methods for screening to identify ligands for GPCRs utilizing these vectors are well suited to drug screening and development of GPCR modulatory agents and compounds. Indeed, the expression vectors and methods described herein are applicable to the identification of GPCR ligands in general, at least in part because the expression vectors can be adapted to incorporate nucleic acid sequences encoding essentially any GPCR and detection of ligand-GPCR interaction in the present screening methods is largely independent of GPCR downstream signaling pathways, which may differ based on the particular GPCR under investigation.

As set forth herein, the present inventors developed a model system in which to explore the role of two GPCRs, Cxcr4b and Cxcr7b, in migration of the primordium during zebrafish development. This system is described in Venkiteswaran et al. (2013, Cell 155:674-687), the entire content of which is incorporated herein by reference. One of the major objectives of the present study was to determine the shape (linear or non-linear), dynamics (presteady-state or steady-state) and generation mechanisms of an attractant gradient in a living animal. Recent descriptions of the distribution of signaling molecules in living animals using overexpression of fluorescently tagged proteins (Entchev et al., 2000; Kicheva et al., 2007; Muller et al., 2012; Teleman and Cohen, 2000; Yu et al., 2009) reported the distribution of the total population of signaling molecules using over expression of tagged molecules, but did not differentiate between the pool of signaling competent and signaling non-competent molecules. To address such unanswered issues, the present inventors sought to assess the distribution of physiological levels of untagged, signaling competent signaling molecules in vivo.

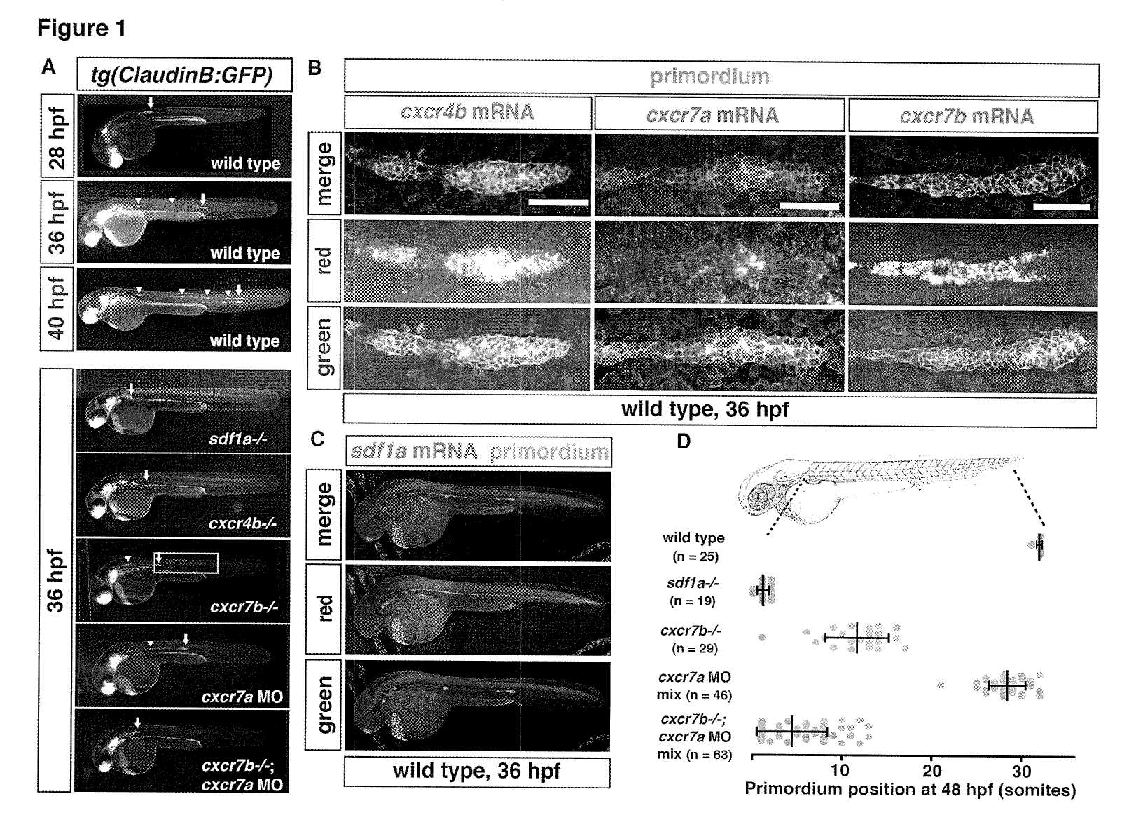

The posterior lateral line primordium in zebrafish is an excellent model for studying the guidance of migrating cells through an attractant (Aman and Piotrowski, 2010). The primordium is composed of about 200 epithelial-like cells that are born behind the ear around 19 hours post fertilization (hpf). Over the next 20 hours, these cells migrate collectively along the body of the fish until they reach the tip of the tail around 40 hpf (FIG. 1A). During this migration period, the primordium deposits 5 to 7 cell clusters bilaterally along the trunk and tail of the embryo (Ghysen and Dambly-Chaudière, 2007). Each of these clusters differentiates into a neuromast, which is a specialized organ that senses water flow around the embryo. The primordium requires the chemokine Sdf1a and its two receptors, Cxcr4b and Cxcr7b, for proper migration (FIG. 1A). The cells of the primordium express cxcr4b (uniformly, FIG. 1B) and cxcr7b (specifically in the rear, FIG. 1B) and migrate over a narrow and uniform stripe of sdf1a mRNA-expressing cells located along the trunk and tail of the embryo (FIG. 1C) (Dambly-Chaudière et al., 2007; David et al., 2002; Valentin et al., 2007). Although chemokine signaling is required for proper migration, it remains unclear how a stripe of uniform sdf1a expression can provide directional guidance to the primordium throughout its journey.

As described herein, the present inventors developed quantitative reporters for Sdf1a protein and Sdf1-signaling and employ quantitative imaging and mathematical modeling to examine the distribution of total Sdf1a protein and signaling competent Sdf1a protein. The present inventors determined that total Sdf1a protein is distributed uniformly along the stripe of chemokine producing cells and underneath the primordium. In contrast, signaling competent Sdf1 protein forms a linear gradient across the primordium throughout its migration with a slope of 7% per cell. Upon abrogation, this gradient re-emerges and reaches steady-state again within 200 minutes. Mathematical modeling shows that the observed gradient kinetics are inconsistent with freely diffusing Sdf1a protein and suggest that the chemokine is hindered in its diffusivity, probably by binding to extracellular molecules.

To determine how the primordium converts a uniform source of Sdf1a protein into an Sdf1-signaling gradient, the expression of Sdf1a protein was analyzed within the primordium. This analysis revealed that the rear of the primordium sequesters 1% of the total Sdf1a protein. This corresponds to 3% or less of the Sdf1a protein available to the primordium. Although controversial (Rajagopal et al., 2009), CXCR7—an alternative receptor for SDF1—has been proposed to act as a chemokine clearance receptor (Boldajipour et al., 2008; Sánchez-Alcañiz et al., 2011). The two CXCR7 orthologs, Cxcr7a and Cxcr7b, are expressed in the rear of the primordium. The present inventors have determined that the two orthologs are required for Sdf1a protein uptake in the rear of the primordium, Sdf1-signaling gradient formation across the primordium, and primordium migration. These observations demonstrate that the primordium sequesters Sdf1a protein in its rear through Cxcr7-mediated chemokine uptake to generate an attractant gradient across itself. This self-generated attractant gradient then provides directional guidance to the migrating primordium. Mathematical modeling shows that this scenario is similar to a dynamic version of Francis Crick's source-sink model, in which a sink moves across a stripe source that provides a constant attractant concentration.

In summary, the present inventors conclude that the signaling competent pool of Sdf1a protein constitutes a small fraction of the total Sdf1a protein, the Sdf1-signaling gradient operates at steady state, and localized Sdf1 protein clearance generates an attractant gradient across the migrating primordium. It is likely that these biophysical and mechanistic insights also apply to other cell migration phenomena during animal development, homeostasis, and disease.

In light of results set forth herein, the present inventors envision GPCR expression constructs and methods of using same to screen for ligands, both native ligands and artificial ligands, capable of modulating GPCR activity. Such GPCR expression constructs comprise a first nucleic acid sequence encoding a GPCR fusion protein comprising a GPCR and a first detectable marker/signal and a second nucleic acid sequence encoding a second polypeptide that is or comprises a second detectable marker/signal and further comprises a membrane localizing sequence. The nucleic acid sequence encoding the GPCR fusion protein and the nucleic acid sequence encoding the second polypeptide are under the transcriptional control of the same promoter and, in one embodiment, are operably linked via an internal ribosomal entry site (IRES). The first and second detectable markers/signals emit distinct detectable signals. Cells encompassing the GPCR expression constructs are also encompassed herein as are methods using same.

Exemplary G protein coupled receptors include, without limitation, CXCR1 (MIM 146929), CXCR2 (MIM 146928), CXCR3 (MIM 300574), CXCR4 (MIM 162643), CXCR5 (MIM 601613), CXCR6 (MIM 605163), CXCR7 (MIM 610376), CCR1 (MIM 601159), CCR2 (MIM 601267), CCR3 (MIM 601268), CCR4 (MIM 604836), CCR5 (MIM 601373), CCR6 (MIM 601835), CCR7 (MIM 600242), CCR8 (MIM 601834), CCR9 (MIM 604738), CCR10 (MIM 600240), CX3CR1 (MIM 601470), GPR15 (MIM 601166), FPR (MIM 136537), D6 (MIM 602648), DARC/Duffy (MIM 613665), CCX-CKR (MIM 606065), PAR1 (MIM 187930), PAR2 (MIM 600933), PAR3 (MIM 601919), PAR4 (MIM 602779) (See, for example, Neel et al., 2005), ADRB1 (MIM 109630), ADRB2 (MIM 109690), ADRB3 (MIM 109691), ADRA1B (MIM 104220), ADRA1D (MIM 104219), ADRA1A (MIM 104221), ADRA2A (MIM 104210) (See, for example, Barak et al., 1994), S1PR1 (MIM 601974), S1PR2 (MIM 605111), S1PR3 (MIM 601965), S1PR4 (MIM 603751), and S1PR5 (MIM 605146) (See, for example, Verzijl et al, 2010). The nucleic and amino acid sequences of the aforementioned GPCRs are all publicly available and can be accessed via the corresponding Online Mendelian Inheritance in Man (MIM) numbers indicated above, the entire content of each of which sequences is incorporated herein by reference. See also Barak et al. (1994). J Biol Chem 269, 2790-2795; Neel et al. (2005). Cytokine & Growth Factor Reviews 16, 637-658; and Verzijl et al. (2010). Mol Cells 29, 99-104 for additional information pertaining to GPCRs, the entire content of each of which is incorporated herein by reference.

Exemplary viral IRESs include, without limitation, the EMCV-R IRES (Jang et al., 1998, J Virol. 62:2636-2643), CrPV IGR IRES (Wilson et al., 2000, Molecular and Cellular Biology 20:4990-4999), HCV type 1a IRES (Tsukiyama-Kohara et al., 1992, Journal of General Virology 73:2313-2318), FMDV type C IRES (Belsham and Brangwyn, 1990, Journal of Virology, 64:5389-5395; Kuhn et al., 1990, J Virol, 64:4625-4631), HAV HM175 IRES (Brown et al., 1994, J Virol, 68:1066-1074), PV type 1 Mahoney IRES (Dorner et al., 1984, J Virol 50:507-514; Pelletier and Sonenberg, 1988, Nature 334:320-325), PV type 3 Leon IRES (Dorner et al., 1984, J Virol 50:507-514; Pelletier and Sonenberg, 1988, Nature 334:320-325), AEV IRES (Bakhshesh et al., Journal of Virology, 2008, 82:1993-2003), CSFV IRES (Rijnbrand et al., 1997, Journal of Virology, 71:451-457), and ERAV 245-961 IRES (Hinton et al., 2000, Journal of Virology, 74:11708-11716). The entire content of each of which is incorporated herein by reference.

Exemplary cellular IRESs include, without limitation, the APAF-1 IRES (Coldwell et al. 2000, Oncogene, 19:899-905; Andreev et al., 2009, Nucleic Acids Res, 37:6135-6147), BCL2 IRES (Van Eden et al., 2004, J Biol Chem, 279:29066-29074), ELH IRES (Dyer et al., 2003, Nat Neurosci, 6:219-220), FMR1 IRES (Chiang et al., 2001, J Biol Chem 276:37916-37921), HSP70 (Rubtsova et al., 2003, The Journal of Biological Chemistry, 278:22350-22356; Andreev et al., 2009, Nucleic Acids Res, 37:6135-6147), Kv1.4 1.2 IRES (Negulescu et al., 1998, The Journal of Biological Chemistry, 273:20109-20113; Kim et al., 2004, Molecular and Cellular Biology, 24:7878-7890), LEF1 IRES (Jiminez et al., 2005, RNA, 11:1385-1399), MTG8a IRES (Mitchell et al., 2005, Genes & Development, 19:1556-1571), MYB IRES (Casini et al., 1995, Oncogene, 11:1019-1026, Mitchell et al., 2005, Genes & Development, 19:1556-1571), and URE2 IRES (Komar et al., 2003, EMBO J, 22:1199-1209). The entire content of each of which is incorporated herein by reference.

All publications referenced above with regard to viral and cellular IRESs are cited in Mokrej\check{s} et al. (2009). IRESite—a tool for the examination of viral and cellular internal ribosome entry sites. Nucleic Acids Research 2010 January; 38(Database issue):D131-136. doi: 10.1093/nar/gkp981. Epub 2009 Nov. 16, the entire content of which is incorporated herein by reference.

Nucleotide sequences of viral and cellular IRESs are as follows:

EMCV-R (SEQ ID NO: 9):
CCCCCCCTCT CCCTCCCCCC CCCCTAACGT TACTGGCCGA
AGCCGCTTGG AATAAGGCCG GTGTGCGTTT GTCTATATGT
TATTTTCCAC CATATTGCCG TCTTTTGGCA ATGTGAGGGC
CCGGAAACCT GGCCCTGTCT TCTTGACGAG CATTCCTAGG
GGTCTTTCCC CTCTCGCCAA AGGAATGCAA GGTCTGTTGA
ATGTCGTGAA GGAAGCAGTT CCTCTGGAAG CTTCTTGAAG
ACAAACAACG TCTGTAGCGA CCCTTTGCAG GCAGCGGAAC
CCCCCACCTG GCGACAGGTG CCTCTGCGGC CAAAAGCCAC
GTGTATAAGA TACACCTGCA AAGGCGGCAC AACCCCAGTG
CCACGTTGTG AGTTGGATAG TTGTGGAAAG AGTCAAATGG
CTCTCCTCAA GCGTATTCAA CAAGGGGCTG AAGGATGCCC
AGAAGGTACC CCATTGTATG GGATCTGATC TGGGGCCTCG
GTGCACATGC TTTACATGTG TTTAGTCGAG GTTAAAAAAC
GTCTAGGCCC CCCGAACCAC GGGGACGTGG TTTTCCTTTG
AAAAACACGA TGATAA
Source: worldwide web site pertaining to
IRES sequences: id = 140.
CrPV IGR (SEQ ID NO: 10):
AAAGCAAAAA TGTGATCTTG CTTGTAAATA CAATTTTGAG
AGGTTAATAA ATTACAAGTA GTGCTATTTT TGTATTTAGG
TTAGCTATTT AGCTTTACGT TCCAGGATGC CTAGTGGCAG
CCCCACAATA TCCAGGAAGC CCTCTCTGCG GTTTTTCAGA
TTAGGTAGTC GAAAAACCTA AGAAATTTAC CT
Source: worldwide web site pertaining to
IRES sequences: id = 40
HCV type 1a (SEQ ID NO: 11):
GCCAGCCCCC TGATGGGGGC GACACTCCAC CATGAATCAC
TCCCCTGTGA GGAACTACTG TCTTCACGCA GAAAGCGTCT
AGCCATGGCG TTAGTATGAG TGTCGTGCAG CCTCCAGGAC
CCCCCCTCCC GGGAGAGCCA TAGTGGTCTG CGGAACCGGT
GAGTACACCG GAATTGCCAG GACGACCGGG TCCTTTCTTG
GATAAACCCG CTCAATGCCT GGAGATTTGG GCGTGCCCCC
GCAAGACTGC TAGCCGAGTA GTGTTGGGTC GCGAAAGGCC
TTGTGGTACT GCCTGATAGG GTGCTTGCGA GTGCCCCGGG
AGGTCTCGTA GACCGTGCAC CATGAGCACG AATCCTAAAC
CTCAAAGAAA AACCAAACGT AAC
Source: worldwide web site pertaining to
IRES sequences: id = 222
FMDV type C (SEQ ID NO: 12):
AGCAGGTTTC CCCAACTGAC ACAAAACGTG CAACTTGAAA
CTCCGCCTGG TCTTTCCAGG TCTAGAGGGG TAACACTTTG
TACTGTGTTT GGCTCCACGC TCGATCCACT GGCGAGTGTT
AGTAACAGCA CTGTTGCTTC GTAGCGGAGC ATGACGGCCG
TGGGAACTCC TCCTTGGTAA CAAGGACCCA CGGGGCCAAA
AGCCACGCCC ACACGGGCCC GTCATGTGTG CAACCCCAGC
ACGGCGACTT TACTGCGAAA CCCACTTTAA AGTGACATTG
AAACTGGTAC CCACACACTG GTGACAGGCT AAGGATGCCC
TTCAGGTACC CCGAGGTAAC ACGCGACACT CGGGATCTGA
GAAGGGGACT GGGGCTTCTA TAAAAGCGCT CGGTTTAAAA
AGCTTCTATG CCTGAATAGG TGACCGGAGG TCGGCACCTT
TCCTTTACAA TTAATGACCC T
Source: worldwide web site pertaining to
IRES sequences: id = 321
HAV HM175 (SEQ ID NO: 13):
TAATTCCTGC AGGTTCAGGG TTCTTAAATC TGTTTCTCTA
TAAGAACACT CATTTTTCAC GCTTTCTGTC TTCTTTCTTC
CAGGGCTCTC CCCTTGCCCT AGGCTCTGGC CGTTGCGCCC
GGCGGGGTCA ACTCCATGAT TAGCATGGAG CTGTAGGAGT
CTAAATTGGG GACACAGATG TTTGGAACGT CACCTTGCAG
TGTTAACTTG GCTTTCATGA ATCTCTTTGA TCTTCCACAA
GGGGTAGGCT ACGGGTGAAA CCTCTTAGGC TAATACTTCT
ATGAAGAGAT GCCTTGGATA GGGTAACAGC GGCGGATATT
GGTGAGTTGT TAAGACAAAA ACCATTCAAC GCCGGAGGAC
TGACTCTCAT CCAGTGGATG CATTGAGTGG ATTGACTGTC
AGGGCTGTCT TTAGGCTTAA TTCCAGACCT CTCTGTGCTT
AGGGCAAACA TCATTTGGCC TTAAATGGGA TTCTGTGAGA
GGGGATCCCT CCATTGACAG CTGGACTGTT CTTTGGGGCC
TTATGTGGTG TTTGCCTCTG AGGTACTCAG GGGCATTTAG
GTTTTTCCTC ATTCTTAAAT AATA
Source: worldwide web site pertaining to
IRES sequences: id = 42
PV type 1 Mahoney (SEQ ID NO: 14):
ATGAGTCTGG ACATCCCTCA CCGGTGACGG TGGTCCAGGC
TGCGTTGGCG GCCTACCTAT GGCTAACGCC ATGGGACGCT
AGTTGTGAAC AAGGTGTGAA GAGCCTATTG AGCTACATAA
GAATCCTCCG GCCCCTGAAT GCGGCTAATC CCAACCTCGG
AGCAGGTGGT CACAAACCAG TGATTGGCCT GTCGTAACGC
GCAAGTCCGT GGCGGAACCG ACTACTTTGG GTGTCCGTGT
TTCCTTTTAT TTTATTGTGG CTGCTTATGG TGACAATCAC
AGATTGTTAT CATAAAGCGA ATTGGATTGG CC
Source: worldwide web site pertaining to
IRES sequences: id = 242
PV type 3 Leon (SEQ ID NO: 15):
TTAAAACAGC TCTGGGGTTG TTCCCACCCC AGAGGCCCAC
GTGGCGGCTA GTACACTGGT ATCACGGTAC CTTTGTACGC
CTGTTTTATA CTCCCTCCCC CGCAACTTAG AAGCATACAA
TTCAAGCTCA ATAGGAGGGG GTGCAAGCCA GCGCCTCCGT
GGGCAAGCAC TACTGTTTCC CCGGTGAGGC CGCATAGACT
GTTCCCACGG TTGAAAGTGG CCGATCCGTT ATCCGCTCAT
GTACTTCGAG AAGCCTAGTA TCGCTCTGGA ATCTTCGACG
CGTTGCGCTC AGCACTCAAC CCCGGAGTGT AGCTTGGGCC
GATGAGTCTG GACAGTCCCC ACTGGCGACA GTGGTCCAGG
CTGCGCTGGC GGCCCACCTG TGGCCCAAAG CCACGGGACG
CTAGTTGTGA ACAGGGTGTG AAGAGCCTAT TGAGCTACAT
GAGAGTCCTC CGGCCCCTGA ATGCGGCTAA TCCTAACCAT
GGAGCAGGCA GCTGCAACCC AGCAGCCAGC CTGTCGTAAC
GCGCAAGTCC GTGGCGGAAC CGACTACTTT GGGTGTCCGT
GTTTCCTTTT ATTCTTGAAT GGCTGCTTAT GGTGACAATC
ATAGATTGTT ATCATAAAGC GAGTTGGATT GGCCATCCAG
TGTGAATCAG ATTAATTACT CCCTTGTTTG TTGGATCCAC
TCCCGAAACG TTTTACTCCT TAACTTATTG AAATTGTTTG
AAGACAGGAT TTCAGTGTCA CA
Source: worldwide web site pertaining to
IRES sequences: id = 598
AEV (SEQ ID NO: 16):
TTTGAAAGAG GCCTCCGGAG TGTCCGGAGG CTCTCTTTCG
ACCCAACCCA TACTGGGGGG TGTGTGGGAC CGTACCTGGA
GTGCACGGTA TATATGCATT CCCGCATGGC AAGGGCGTGC
TACCTTGCCC CTTGACGCAT GGTATGCGTC ATCATTTGCC
TTGGTTAAGC CCCATAGAAA CGAGGCGTCA CGTGCCGAAA
ATCCCTTTGC GTTTCACAGA ACCATCCTAA CCATGGGTGT
AGTATGGGAA TCGTGTATGG GGATGATTAG GATCTCTCGT
AGAGGGATAG GTGTGCCATT CAAATCCAGG GAGTACTCTG
GCTCTGACAT TGGGACATTT GATGTAACCG GACCTGGTTC
AGTATCCGGG TTGTCCTGTA TTGTTACGGT GTATCCGTCT
TGGCACACTG AAAGGGTATT TTTGGGTAAT CCTTTCCTAC
TGCCTGATAG GGTGGCGTGC CCGGCCACGA GAGATTAAGG
GTAGCAATTT AAAC
Source: worldwide web site pertaining to
IRES sequences: id = 416
CSFV (SEQ ID NO: 17):
GTATACGAGG TTAGTTCATT CTCGTATACA CGATTGGACA
AATCAAAATT ATAATTTGGT TCAGGGCCTC CCTCCAGCGA
CGGCCGAACT GGGCTAGCCA TGCCCATAGT AGGACTAGCA
AAACGGAGGG ACTAGCCATA GTGGCGAGCT CCCTGGGTGG
TCTAAGTCCT GAGTACAGGA CAGTCGTCAG TAGTTCGACG
TGAGCAGAAG CCCACCTCGA GATGCTACGT GGACGAGGGC
ATGCCAAGAC ACACCTTAAC CCTAGCGGGG GTCGCTAGGG
TGAAATCACA CCACGTGATG GGAGTACGAC CTGATAGGGC
GCTGCAGAGG CCCACTATTA GGCTAGTATA AAAATCTCTG
CTGTACATGG CAC
Source: worldwide web site pertaining to
IRES sequences: id = 148
ERAV (SEQ ID NO: 18):
GAGAGGAGCC CGTTTTCGGG CACTTGTCTC CTAAACAATG
TTGGCGCGCA TTTGCGCGCC CCCCCCCTTT TTCAGCCCCC
TGTCATTGAC TGGTCGAAGC GTTCGCAATA AGACTGGTCG
TCACTTGGCT GTTCTATCGT TTCAGGCTTT AGCGCGCCCT
TGCGCGGCGG GCCGTCAAGC CCGTGCGCTG TATAGCGCCA
GGTAACCGGA CAGCGGCGTG CTGGATTTTC CCGGTGCCAT
TGCTCTGGAT GGTGTCACCA AGCTGACAAA TGCGGAGTGA
ACCTCACAAA GCGACACGCC TGTGGTAGCG CTGCCCAAAA
GGGAGCGGAA CTCCCCGCCG AGGCGGTCCT CTCTGGCCAA
AAGCCCAGCG TTGATAGCGC CTTTTGGGAT GCAGGAACCC
CACCTGCCAG GTGTGAAGTG GAGTGAGCGG ATCTCCAATT
TGGTCTGTTC TGAACTACAC CATTTACTGC TGTGAAGAAT
GCCCTGGAGG CAAGCTGGTT ACAGCCCTGA CCAGGCCCTG
CCCGTGACTC TCGACCGGCG CAGGGTCAAA AATTGTCTAA
GCAGCAGCAG GAACGCGGGA GCGTTTCTTT TCCTTTTGTA
CTGACATGAT GGCGGCGTCT AAGGTGTATA GAGTTTGCGA
GCAGACTCTG CTGGCAGGTG CCGTTCGCAT GATGGACAAA
TTCTTGCAAA AGAGAACTGT TTTTGTCCCC CA
Source: worldwide web site pertaining to
IRES sequences: id = 286
APAF-1 (SEQ ID NO: 19):
CGGCTTGAGG CAGAGACCAG GAGGCAGCTA GAGGAGCAGA
CGTCTCACTC CGCTCGCGGA AGGGTGTGAG AGGGGTGTGT
GGGGGTCGGC AGCGAGGGGT GTGTGCCATC AGCCACCGGC
GACGATCTGA GACAGTCGCA GCGGCTTTCC GAGCGGCGTC
CGCTTCCCGC CCGGGCAGCT CCCGCCAGAG GGGTGAAGCG
GCGACTGGAG TGGCCGTGCT TTTGTGCCCT GGGTCCCGGT
ACCCTCCCCT GGTGCGGCCC GAGGCAAGCC CACCGAGGTG
ACCACCCCTC GACGCCGCTT GGAGATCCCG GGCATCCACC
CTGCGCCCCG AGCAGCTGAT ACCCAGGGAG GTGTCAGGAC
CTGCCCGGGG CGCGGGGTCG CCGGAAGCCA GGCGGGAGCC
CCGGCTGCTT TCTGGCAATC TAGTCTCATA AGTGACCCTC
CCTGGGCTGC TTTCTTTCGA TTATCATCAG TGACCCTACC
CCGGCTGCTC TTCCCAGCAC AACTCCGGTG CAAAGGCTTG
GGCATCCTGG TGCTTTGCCT CTAGCCCATG CTCCACAGCG
AGGAGAGAGA AAACCCTGAG GCA
Source: worldwide web site pertaining to
IRES sequences: id = 342
BCL2 (SEQ ID NO: 20):
AAAAAATAAA ACCCTCCCCC ACCACCTCCT TCTCCCCACC
CCTCGCCGCA CCACACACAG CGCGGGCTTC TAGCGCTCGG
CACCGGCGGG CCAGGCGCGT CCTGCCTTCA TTTATCCAGC
AGCTTTTCGG AAAATGCATT TGCTGTTCGG AGTTTAATCA
GAAGACGATT CCTGCCTCCG TCCCCGGCTC CTTCATCGTC
CCATCTCCCC TGTCTCTCTC CTGGGGAGGC GTGAAGCGGT
CCCGTGGATA GAGATTCATG CCTGTGTCCG CGCGTGTGTG
CGCGCGTATA AATTGCCGAG AAGGGGAAAA CATCACAGGA
CTTCTGCGAA TACCGGACTG AAAATTGTAA TTCATCTGCC
GCCGCCGCTG CCAAAAAAAA ACTCGAGCTC TTGAGATCTC
CGGTTGGGAT TCCTGCGGAT TGACATTTCT GTGAAGCAGA
AGTCTGGGAA TCGATCTGGA AATCCTCCTA ATTTTTACTC
CCTCTCCCCC CGACTCCTGA TTCATTGGGA AGTTTCAAAT
CAGCTATAAC TGGAGAGTGC TGAAGATTGA TGGGATCGTT
GCCTTATGCA TTTGTTTTGG TTTTACAAAA AGGAAACTTG
ACAGAGGATC ATGCTGTACT TAAAAAATAC AAGTAAGTCT
CGCACAGGAA ATTGGTTTAA TGTAACTTTC AATGGAAACC
TTTGAGATTT TTTACTTAAA GTGCATTCGA GTAAATTTAA
TTTCCAGGCA GCTTAATACA TTGTTTTTAG CCGTGTTACT
TGTAGTGTGT ATGCCCTGCT TTCACTCAGT GTGTACAGGG
AAACGCACCT GATTTTTTAC TTATTAGTTT GTTTTTTCTT
TAACCTTTCA GCATCACAGA GGAAGTAGAC TGATATTAAC
AATACTTACT AATAATAACG TGCCTCATGA AATAAAGATC
CGAAAGGAAT TGGAATAAAA ATTTCCTGCG TCTCATGCCA
AGAGGGAAAC ACCAGAATCA AGTGTTCCGC GTGATTGAAG
ACACCCCCTC GTCCAAGAAT GCAAAGCACA TCCAATAAAA
TAGCTGGATT ATAACTCCTC TTCTTTCTCT GGGGGCCGTG
GGGTGGGAGC TGGGGCGAGA GGTGCCGTTG GCCCCCGTTG
CTTTTCCTCT GGGAAGG
Source: worldwide web site pertaining to
IRES sequences: id = 103
ELH (SEQ ID NO: 21):
CTTTACCGAC AACGTCCAGC GGTCCAGTCG AGAAGTAAGT
AGTCCGCGCT GCGAGCATAA CTCTACCAGA GAAACGCGCA
CTTTATTGGT CAAGACAGAC GGAAGCCGGC GGTTGGACGC
AAAGGTTGAC TCGGAAGCTG AGAGATTGTT GAGTAACACG
CAGAAGTCTT GAAAGTATCA AGTATTGAAC ATATTTCAAG
GGACTTGGTT TCGGTGAAGT CGTCAATTCC TTTTATCGTC
AACGTTTCCA CAGCCCTCAG AATAGAAATT TCCAACAAGC
CAAAGCCTAC GTAATGAAGC GCCCCAATAA CCGGCCGAC
Source: worldwide web site pertaining to
IRES sequences: id = 241
FMR1 (SEQ ID NO: 22):
GGCCTCAGTC AGGCCTCAGC TCCGTTTCGG TTTCACTTCC
GGTGGAGGGC CGCCTCTGAG CGGGCGGCGG GCCGACGGCG
AGCGCGGGCG GCGGCGGTGA CGGAGGCGCC GCTGCCAGGG
GGCGTGCGGC AGCGCGGCGG CGGCGGCGGC GGCGGCGGCG
GCGGCGGCGG CGGCGGCGGC GGCTGGGCCT CGAGCGCCCG
CAGCCCACCT CTCGGGGGCG GGCTCCCGGC GCTAGCAGGG
CTGAAGAGAA CA
Source: worldwide web site pertaining to
IRES sequences: id = 122
HSP70 (SEQ ID NO: 23):
CTGCGACAGT CCACTACCTT TTTCGAGAGT GACTCCCGTT
GTCCCAAGGC TTCCCAGAGC GAACCTGTGC GGCTGCAGGC
ACCGGCGCGT CGAGTTTCCG GCGTCCGGAA GGACCGAGCT
CTTCTCGCGG ATCCAGTGTT CCGTTTCCAG CCCCCAATCT
CAGAGCGGAG CCGACAGAGA GCAGGGAACC GGC
Source: worldwide web site pertaining to
IRES sequences: id = 118
Kv1.4 1.2 (SEQ ID NO: 24):
ACTGGGATG GATACTGGAG AAGGAATGCA GGCTTAACAA
GTGATCGCTG CTGTCTAGGA TTTTGAGTCT TTTTCGGAGA
ACCTTGACTT CCGTTCCCAG CCCATGTCTG CTGTGCCGAA
CTCCAGAGGA ACCAGAAATC TCCGGGGTCT ACCTTGGGGC
GTCCCCAATC TCCACCTCTG GGCTCCAGTA ACGAGGACTC
TGCAATACCC CCTAGCCCCC TGGCCAAGAC AACCGAACTT
GTTCCGTGGA TATTTGGGAT CCTCCACCTG CCAAACCTGA
GCGATTTTTT TTGTACTGCG CCCCCACCCC CAATGATTCT
GCCCCTCCTC CAGCTGTTGC AGCGTGGAAA AGGGGAAACA
AATCACCGGG GGGGATTTTT TTCGTCTATT TTTATTTTTC
GCACTTGCTG GGAATGGTGA AGTGCTTCTT GTGAATGATT
CTAGCCAAAG GATGCTCTTC ATTTCCTGCT TTCTATGGAG
ACCTCAGTGT TGAGTTTGCC TCTGCTGGAA CTGCGTCTAC
CACCTTCTCT ACCTTCCAAG GTCTTTGCCT CTATCTACAA
CCTGGCATTG TCTGTGGGTC CATGAAGGCT TGGATGACCT
TAGAGGGAAG GTCTGGGAGT CCACCCTCAT AGACTAAGCA
GCAATGGCTG GGCATATTTT AAGCCGCATT TTAACATGGG
TCAAGCCATC AGTAGAAGGC AAGTGCTAAG ACTAAAGACT
TATTTGAATT TTATTTAAAT TAGATGGACT GGGCCTTGGC
CAATTTCCAT GCAAGAAAAA GTATATTTCA TTTTCTAGGC
ACAACTTCTG AGTGTCAGAT ACTTGCTGTC TTTGAGTCTT
GTGGCGTCAT CACCGGACAG CATCCCAGAC AGACTTCCAG
ATTTGAACAT CTACCCCCCA ACACGTAGGT GTATGGGAGA
CCACATCATT TCATGACTTA TGTTTGAGGA ACACTAGGCT
GTTGTCTAGA CGAGGCAAGC TCTGGAAAGC AACGCCGAGT
CTCTGAGAAG AGGGAGCATA GGCTGTGCTG ATTTAAAAAC
AGAAAATGCA AAGTTGGACT GAAAATATCC CACGTCTTCT
AAGCAATCTG CTTAAGGCTT CCAAACTTAC CTTAATTTGG
TAAGAAAATA AGCTGCCCTA TTTTTCTTTC TTCTTCTCTT
ACAACTGGAA GCAGCCATTT CCCCAAACCA CCACCAT
Source: worldwide web site pertaining to
IRES sequences: id = 124
LEF1 (SEQ ID NO: 25):
GCGCGGGAGG AGGAGAAGCA GTGGGGAGGC
GCAGCCGCTC ACCTGCGGGG CAGGGCGCGG AGGAGGGACC
CGGTGCGCGC TCTCGGGCCG AGGAACCAGG ACGCGCCCGG
AGCCTCGCAC GCGGCCAAGC TCGGGCGTCC CCTCCCCTCG
GCCGGGCGAA CTCAAGGGGC GCAGCTCTTT GCTTTGACAG
AGCTGGCCGG CGGAGGCGTG CAGAGCGGCG AGCCGGCGAG
CCAGGCTGAG AAACTCGACC CGGGAACAAA GAGGGGTCGG
ACTGAGTGTG TGTGTCGGCT CGAGCTCCGG GAGAGGCATT
TGCCCGAGGC CCCGCTGTGA CTCCCCGAGA CTCCGCAGTG
CCCTCCACTG GGAGTCCCCG CGCTTGCCGG AAAAACTTTA
TTCTTGGCAA ACTTCTCTTT CTCTTCCCCT CCTCCTCGGC
CCCCATCTTC TGCTCCTCCT CCTTCTCTAG CAGATTAAAT
GAGCCTCGAG AAGAAAAACC GAAGCGAAAG GGAAGAAAAT
AAGAAGATCT AAAACGGACA TCTCCAGCGT GGGTGGCTCC
TTTTTCTTTT TCTTTTTTTC CCACCCTTCA GGAAGTGGAC
GTTTCGTTAT CTTCTGATCC TTGCACCTTC TTTTGGGGCA
AACGGGGCCC TTCTGCCCAG ATCCCCTCTC TTTTCTCGGA
AAACAAACTA CTAAGTCGGC ATCCGGGGTA ACTACAGTGG
AGAGGGTTTC CGCGGAGACG CGCCGCCGGA CCCTCCTCTG
CACTTTGGGG AGGCGTGCTC CCTCCAGAAC CGGCGTTCTC
CGCGCGCAAA TCCCGGCGAC GCGGGGTCGC GGGGTGGCCG
CCGGGGCAGC CTCGTCTAGC GCGCGCCGCG CAGACGCCCC
CGGAGTCGCC AGCTACCGCA GCCCTCGCCG CCCAGTGCCC
TTCGGCCTCG GGGCGGGCGC CTGCGTCGGT CTCCGCGAAG
CGGGAAAGCG CGGCGGCCGC CGGGATTCGG GCGCCGCGGC
AGCTGCTCCG GCTGCCGGCC GGCGGCCCCG CGCTCGCCCG
CCCCGCTTCC GCCCGCTGTC CTGCTGCACG AACCCTTCCA
ACTCTCCTTT CCTCCCCCAC CCTTGAGTTA CCCCTCTGTC
TTTCCTGCTG TTGCGCGGGT GCTCCCACAG CGGAGCGGAG
ATTACAGAGC CGCCGGG
Source: worldwide web site pertaining to
IRES sequences: id = 220
MTG8a (SEQ ID NO: 26):
GCAATCCGGA GTGTCAGCTC TCCATCTGTC TCTGCCTGGC
AGGCGCACGC GCCCAGCACC CTGCCTCCGG CGATGCCGCC
CCAGCCCCTC TGATGGCCCT CCTCTCTGCT GCCACTCATT
CCAGAACAGG AGGCATGAGC CCGGAACGCG CTTGCTTTTA
GGAGACAGCC ACTTTCTGTG TGGTACGCTG GATTCAAGG
Source: worldwide web site pertaining to
IRES sequences: id = 474
MYB (SEQ ID NO: 27):
ATATCAACCT GTTTCCTCCT CCTCCTTCTC CTCCTCCTCC
GTGACCTCCT CCTCCTCTTT CTCCTGAGAA ACTTCGCCCC
AGCGGTGCGG AGCGCCGCTG CGCAGCCGGG GAGGGACGCA
GGCAGGCGGCGGGCAGCGGGAGGCGGCAGC
Source: worldwide web site pertaining to
IRES sequences: id = 471
URE2 (SEQ ID NO: 28):
AGCCAAAATA ATGATAACGA GAATAATATC AAGAATACCT
TAGAACAACA TCGACAACAA CAACAGGCAT TTTCGGATAT
GAGTCACGTG GAGTATTCCA GAATTACAAA ATTTTTTCAA
GAACAACCAC TGGAGGGATA TACCCTTTTC TCTCACAGGT
CTGCGCC
Source: worldwide web site pertaining to
IRES sequences: id = 115

Suitable fluorescent marker pairs comprise those that are distinguishable and, more particularly, distinguishable by fluorescence in assay systems described herein. Essentially any combination will work as long as they are distinguishable by fluorescence, for example, cerulean fluorescent protein (CFP) and yellow fluorescent protein (YFP), YFP and CFP, green fluorescent protein (GFP) and red fluorescent protein (RFP), RFP and GFP, RFP and near-infrared fluorescent protein (iRFP). The aforementioned fluorescent proteins and others are known in the art and described in, for example, Shaner et al. 2005, Nature Methods 2:905-909; Matz et al. 1999, Nat Biotechnol 17: 969-973: Kallal et al. Trends in Pharmacology 2000, 21:175-180; Filonov et al. 2011, Nature Biotechnology 29:757-761, the entire content of each of which is incorporated herein by reference.

For each of the following fluorescent proteins, the nucleic acid sequence is followed by the amino acid sequence.

CFP (SEQ ID NO: 29):
ATGTCTCTTTCAAAGCATGGCATCACACAAGAAATGCCGACGAAATACCATATGAAAG
GCAGTGTCAATGGCCATGAATTCGAGATCGAAGGTGTAGGAACTGGACACCCTTACGA
AGGGACACACATGGCCGAATTAGTGATCATAAAGCCTGCGGGAAAACCCCTTCCATTCT
CCTTTGACATACTGTCAACAGTCATTCAATACGGAAACAGATGCTTCACTAAGTACCCT
GCAGACCTGCCTGACTATTTCAAGCAAGCATACCCAGGTGGAATGTCATATGAAAGGTC
ATTTGTGTATCAGGATGGAGGAATTGCTACAGCGAGCTGGAACGTTGGTCTCGAGGGAA
ATTGCTTCATCCACAAATCCACCTATCTTGGTGTAAACTTTCCTGCTGATGGACCCGTAA
TGACAAAGAAGACAATTGGCTGGGATAAAGCCTTTGAAAAAATGACTGGGTTCAATGA
GGTGTTAAGAGGTGATGTGACTGAGTTTCTTATGCTCGAAGGAGGTGGTTACCATTCAT
GCCAGTTTCACTCCACTTACAAACCAGAGAAGCCGGTCGAACTGCCCCCGAATCATGTC
ATAGAACATCACATTGTGAGGACCGACCTTGGCAAGACTGCAAAAGGCTTCATGGTCA
AGCTGGTACAACATGCTGCGGCTCATGTTAACCCTTTGAAGGTTCAATAA
CFP (SEQ ID NO: 30):
MSLSKHGITQEMPTKYHMKGSVNGHEFEIEGVGTGHPYEGTHMA
ELVIIKPAGKPLPFSFDILSTVIQYGNRCFTKYPADLPDYFKQAYP
GGMSYERSFVYQDGGIATASWNVGLEGNCFIHKSTYLGVNFPAD
GPVMTKKTIGWDKAFEKMTGFNEVLRGDVTEFLMLEGGGYHSC
QFHSTYKPEKPVELPPNHVIEHHIVRTDLGKTAKGFMVKLVQHA
AAHVNPLKVQ
YFP (SEQ ID NO: 31):
ATGGTGAGCAAGGGCGAGGAGCTGTTCACCGGGGTGGTGCCCATCCTGGTCGAGCTGG
ACGGCGACGTAAACGGCCACAAGTTCAGCGTGTCCGGCGAGGGCGAGGGCGATGCCAC
CTACGGCAAGCTGACCCTGAAGTTCATCTGCACCACCGGCAAGCTGCCCGTGCCCTGGC
CCACCCTCGTGACCACCTTCGGCTACGGCCTGCAGTGCTTCGCCCGCTACCCCGACCAC
ATGAAGCAGCACGACTTCTTCAAGTCCGCCATGCCCGAAGGCTACGTCCAGGAGCGCA
CCATCTTCTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGACGGCAAC
ATCCTGGGGCACAAGCTGGAGTACAACTACAACAGCCACAACGTCTATATCATGGCCG
ACAAGCAGAAGAACGGCATCAAGGTGAACTTCAAGATCCGCCACAACATCGAGGACGG
CAGCGTGCAGCTCGCCGACCACTACCAGCAGAACACCCCCATCGGCGACGGCCCCGTG
CTGCTGCCCGACAACCACTACCTGAGCTACCAGTCCGCCCTGAGCAAAGACCCCAACGA
GAAGCGCGATCACATGGTCCTGCTGGAGTTCGTGACCGCCGCCGGGATCACTCTCGGCA
TGGACGAGCTGTACAAGTAA
YFP (SEQ ID NO: 32):
MVSKGEELFTGVVPILVELDGDVNGHKFSVSGEGEGDATYGKLT
LKFICTTGKLPVPWPTLVTTFGYGLQCFARYPDHMKQHDFFKSA
MPEGYVQERTIFFKDDGNYKTRAEVKFEGDTLVNRIELKGIDFKE
DGNILGHKLEYNYNSHNVYIMADKQKNGIKVNFKIRHNIEDGSV
QLADHYQQNTPIGDGPVLLPDNHYLSYQSALSKDPNEKRDHMVL
LEFVTAAGITLGMDELYK
GFP (SEQ ID NO: 33):
ATGGTGAGCAAGGGCGAGGAGCTGTTCACCGGGGIGGTGCCCATCCTGGTCGAGCTGG
ACGGCGACGTAAACGGCCACAAGTTCAGCGTGTCCGGCGAGGGCGAGGGCGATGCCAC
CTACGGCAAGCTGACCCTGAAGTTCATCTGCACCACCGGCAACTGCCCGTGCCCTGGCC
CACCCTCGTGACCACCCTGACCTACGGCGTGCAGTGCTTCAGCCGCTACCCCGACCACA
TGAAGCAGCACGACTTCTTCAAGTCCGCCATGCCCGAAGGCTACGTCCAGGAGCGCACC
ATCTTCTTCAAGGACGACGGCAACTACAAGACCCGCGCCGAGGTGAAGTTCGAGGGCG
ACACCCTGGTGAACCGCATCGAGCTGAAGGGCATCGACTTCAAGGAGGACGGCAACAT
CCTGGGGCACAAGCTGGAGTACAACTACAACAGCCACAACGTCTATATCATGGCCGAC
AAGCAGAAGAACGGCATCAAGGTGAACTTCAAGATCCGCCACAACATCGAGGACGGCA
GCGTGCAGCTCGCCGACCACTACCAGCAGAACACCCCCATCGGCGACGGCCCCGTGCT
GCTGCCCGACAACCACTACCTGAGCACCCAGTCCGCCCTGAGCAAAGACCCCAACGAG
AAGCGCGATCACATGGTCCTGCTGGAGTTCGTGACCGCCGCCGGGATCACTCTCGGCAT
GGACGAGCTGTACAAGTAA
GFP (SEQ ID NO: 34):
MVSKGEELFTGVVPILVELDGDVNGHKFSVSGEGEGDATYGKLT
LKFICTTGKLPVPWPTLVTTLTYGVQCFSRYPDHMKQHDFFKSA
MPEGYVQERTIFFKDDGNYKTRAEVKFEGDTLVNRIELKGIDFKE
DGNILGHKLEYNYNSHNVYIMADKQKNGIKVNFKIRHNIEDGSV
QLADHYQQNTPIGDGPVLLPDNHYLSTQSALSKDPNEKRDHMVL
LEFVTAAGITLGMDELYK
RFP (SEQ ID NO: 35):
ATGGTGAGCAAGGGCGAGGAGGATAACATGGCCATCATCAAGGAGTTCATGCGCTTCA
AGGTGCACATGGAGGGCTCCGTGAACGGCCACGAGTTCGAGATCGAGGGCGAGGGCGA
GGGCCGCCCCTACGAGGGCACCCAGACCGCCAAGCTGAAGGTGACCAAGGGTGGCCCC
CTGCCCTTCGCCTGGGACATCCTGTCCCCTCAGTTCATGTACGGCTCCAAGGCCTACGTG
AAGCACCCCGCCGACATCCCCGACTACTTGAAGCTGTCCTTCCCCGAGGGCTTCAAGTG
GGAGCGCGTGATGAACTTCGAGGACGGCGGCGTGGTGACCGTGACCCAGGACTCCTCC
CTGCAGGACGGCGAGTTCATCTACAAGGTGAAGCTGCGCGGCACCAACTTCCCCTCCGA
CGGCCCCGTAATGCAGAAGAAGACCATGGGCTGGGAGGCCTCCTCCGAGCGGATGTAC
CCCGAGGACGGCGCCCTGAAGGGCGAGATCAAGCAGAGGCTGAAGCTGAAGGACGGC
GGCCACTACGACGCTGAGGTCAAGACCACCTACAAGGCCAAGAAGCCCGTGCAGCTGC
CCGGCGCCTACAACGTCAACATCAAGTTGGACATCACCTCCCACAACGAGGACTACACC
ATCGTGGAACAGTACGAACGCGCCGAGGGCCGCCACTCCACCGGCGGCATGGACGAGC
TGTACAAGTAA
RFP (SEQ ID NO: 36):
MVSKGEEDNMAIIKEFMRFKVHMEGSVNGHEFEIEGEGEGRPYE
GTQTAKLKVTKGGPLPFAWDILSPQFMYGSKAYVKHPADIPDYL
KLSFPEGFKWERVMNFEDGGVVTVTQDSSLQDGEFIYKVKLRGT
NFPSDGPVMQKKTMGWEASSERMYPEDGALKGEIKQRLKLKDG
GHYDAEVKTTYKAKKPVQLPGAYNVNIKLDITSHNEDYTIVEQY
ERAEGRHSTGGMDELYK
iRFP (SEQ ID NO: 37):
ATGGCGGAAGGATCCGTCGCCAGGCAGCCTGACCTCTTGACCTGCGACGATGAGCCGA
TCCATATCCCCGGTGCCATCCAACCGCATGGACTGCTGCTCGCCCTCGCCGCCGACATG
ACGATCGTTGCCGGCAGCGACAACCTTCCCGAACTCACCGGACTGGCGATCGGCGCCCT
GATCGGCCGCTCTGCGGCCGATGTCTTCGACTCGGAGACGCACAACCGTCTGACGATCG
CCTTGGCCGAGCCCGGGGCGGCCGTCGGAGCACCGATCACTGTCGGCTTCACGATGCGA
AAGGACGCAGGCTTCATCGGCTCCTGGCATCGCCATGATCAGCTCATCTTCCTCGAGCT
CGAGCCTCCCCAGCGGGACGTCGCCGAGCCGCAGGCGTTCTTCCGCCGCACCAACAGC
GCCATCCGCCGCCTGCAGGCCGCCGAAACCTTGGAAAGCGCCTGCGCCGCCGCGGCGC
AAGAGGTGCGGAAGATTACCGGCTTCGATCGGGTGATGATCTATCGCTTCGCCTCCGAC
TTCAGCGGCGAAGTGATCGCAGAGGATCGGTGCGCCGAGGTCGAGTCAAAACTAGGCC
TGCACTATCCTGCCTCAACCGTGCCGGCGCAGGCCCGTCGGCTCTATACCATCAACCCG
GTACGGATCATTCCCGATATCAATTATCGGCCGGTGCCGGTCACCCCAGACCTCAATCC
GGTCACCGGGCGGCCGATTGATCTTAGCTTCGCCATCCTGCGCAGCGTCTCGCCCGTCC
ATCTGGAATTCATGCGCAACATAGGCATGCACGGCACGATGTCGATCTCGATTTTGCGC
GGCGAGCGACTGTGGGGATTGATCGTTTGCCATCACCGAACGCCGTACTACGTCGATCT
CGATGGCCGCCAAGCCTGCGAGCTAGTCGCCCAGGTTCTGGCCTGGCAGATCGGCGTGA
TGGAAGAG
iRFP (SEQ ID NO: 38):
MAEGSVARQPDLLTCDDEPIHIPGAIQPHGLLLALAADMTIVAGS
DNLPELTGLAIGALIGRSAADVFDSETHNRLTIALAEPGAAVGAPI
TVGFTMRKDAGFIGSWHRHDQLIFLELEPPQRDVAEPQAFFRRTN
SAIRRLQAAETLESACAAAAQEVRKITGFDRVMIYRFASDFSGEV
IAEDRCAEVESKLGLHYPASTVPAQARRLYTINPVRIIPDINYRPV
PVTPDLNPVTGRPIDLSFAILRSVSPVHLEFMRNIGMHGTMSISIL
RGERLWGLIVCHHRTPYYVDLDGRQACELVAQVLAWQIGVMEE
Kate2, which is also known as TagRFP2 and is an
RFP variant (SEQ ID NO: 39):
ATGGTGTCTAAGGGCGAAGAGCTGATTAAGGAGAACATGCACATGAAGCTGTACATGG
AGGGCACCGTGAACAACCACCACTTCAAGTGCACATCCGAGGGCGAAGGCAAGCCCTA
CGAGGGCACCCAGACCATGAGAATCAAGGCCGTCGAGGGCGGCCCTCTCCCCTTCGCCT
TCGACATCCTGGCTACCAGCTTCATGTACGGCAGCAAAACCTTCATCAACCACACCCAG
GGCATCCCCGACTTCTTTAAGCAGTCCTTCCCTGAGGGCTTCACATGGGAGAGAGTCAC
CACATACGAAGACGGGGGCGTGCTGACCGCTACCCAGGACACCAGCCTCCAGGACGGC
TGCCTCATCTACAACGTCAAGATCAGAGGGGTGAACTTCCCATCCAACGGCCCTGTGAT
GCAGAAGAAAACACTCGGCTGGGAGGCCTCCACCGAGACCCTGTACCCCGCTGACGGC
GGCCTGGAAGGCAGAGCCGACATGGCCCTGAAGCTCGTGGGCGGGGGCCACCTGATCT
GCAACTTGAAGACCACATACAGATCCAAGAAACCCGCTAAGAACCTCAAGATGCCCGG
CGTCTACTATGTGGACAGAAGACTGGAAAGAATCAAGGAGGCCGACAAAGAGACCTAC
GTCGAGCAGCACGAGGTGGCTGTGGCCAGATACTGCGACCTCCCTAGCAAACTGGGGC
ACAAACTTAATTGA
Kate2, which is also known as TagRFP2 and is an
RFP variant (SEQ ID NO: 40):
MVSKGEELIKENMHMKLYMEGTVNNHHFKCTSEGEGKPYEGTQTMRIKAVEGGPLPFAFD
ILATSFMYGSKTFINHTQGIPDFFKQSFPEGFTWERVTTYEDGGVLTATQDTSLQDGCLI
YNVKIRGVNFPSNGPVMQKKTLGWEASTETLYPADGGLEGRADMALKLVGGGHLICNL
KTTYRSKKPAKNLKMPGVYYVDRRLERIKEADKETYVEQHEVAVARYCDLPSKLGHKLN

Exemplary promoters envisioned for incorporation into GPCR expression constructs are known in the art and include, without limitation, the simian virus 40 early promoter (SV40), cytomegalovirus immediate-early promoter (CMV), human Ubiquitin C promoter (UBC), human elongation factor 1α promoter (EF1A), mouse phosphoglycerate kinase 1 promoter (PGK), chicken β-Actin promoter coupled with CMV early enhancer (CAGG), human β-Actin promoter (ACTB), and human phosphoglycerate kinase promoter (PGK). These promoters are commonly used in mammalian systems. Two promoters that are commonly used in Drosophila systems are the copia transposon promoter (COPIA) and actin 5C promoter (ACT5C). See also Qin et al. (2010, PLoS ONE 5:e10611 and Norrman et al. (2010, PLoS ONE 5:e12413), the entire content of each of which is incorporated herein by reference.

Exemplary libraries for screening using methods described herein include, without limitation, Chembridge Corporation Screening compounds database, Sigma-Aldrich Bioactive small molecules Database, Calbiochem Inhibitors, NIH molecular libraries, Broad Institute small molecule Profiling, Selleckchem library, and libraries available from Otava, Phoenix Pharmaceuticals, Inc., and Funxional Therapeutics.

In order to more clearly set forth the parameters of the present invention, the following definitions are used:

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure.

The term “complementary” refers to two DNA strands that exhibit substantial normal base pairing characteristics. Complementary DNA may, however, contain one or more mismatches. The term “hybridization” refers to the hydrogen bonding that occurs between two complementary DNA strands.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 60%, but often, more than 85%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

In accordance with methods described herein, a mutant GPCR may be used in the screening assays described herein to identify an inhibitor, activator, or ligand of the mutant GPCR. In a particular scenario, a mutant GPCR having abnormal GPCR activity that is associated with a disease or condition can be used in screening assays described herein to identify a compound or agent that partially or completely restores GPCR activity to the mutant GPCR. In a particular aspect, screening assays may be performed in parallel using the wildtype GPCR, so as to identify a compound that acts predominantly on the mutant GPCR and does not alter corresponding wildtype GPCR activity. In a further scenario, if a mutant GPCR has impaired or reduced GPCR activity relative to the corresponding wildtype GPCR, screening assays can be performed to identify a compound or agent that activates GPCR activity of the mutant GPCR.

Mutations in GPCRs that result in gain or loss of function, moreover, are associated with certain human diseases. GPCR polymorphisms, for example, have been linked to hypertension, idiopathic cardiomyopathy (endothelin receptor), autosomal dominant hypocalcemia and familial hypocalciuric hypercalcemia (calcium-sensing receptor), follicular maturation arrest and suppression of spermatogenesis (follicle stimulating hormone receptor), and bronchodilator desensitization and nocturnal asthma (β₂-adrenoreceptors). See, for example, Cacace et al. Drug Discovery Today 8:785-792, 2003; Huhtaniemi et al. Hormone Res 53:9-16, 2000; Perez et al. Receptors Channels 8:57-64, 2002.

Other examples of diseases or disease resistance associated with mutant GPCRs include: WHIM syndrome (inability to downregulate CXCR4 after stimulation), retinitis pigmentosa and congenital night blindness (consitutively active Rhodopsin), Leydig's cell hyperplasia (constitutively active Luteininzing hormone/chorionic gonadotropin receptor), ovarian dysgenesis (Follicle-stimulating hormone receptor has decreased affinity for ligand), monogenic form of binge eating resulting in morbid obesity (Melanocortin 4 receptor), Hirschsprung's disease (reduced G_{q} coupling to Endothelin receptor, type B in vitro), isolated glucocorticoid deficiency (Adrenocorticotropin receptor altered/loss of function), adrenocortical tumors (reduced expression of Adrenocorticotropin receptor), idiopathic hypogonadotropic hypogonadism (reduction or loss of Gonadotropin-releasing hormone receptor function), Jansen's metaphyseal chondrodysplasia (constitutively active Gonadotropin-releasing hormone receptor), Blomstrand's chondrodysplasia (no accumulation of cAMP in response to Parathyroid hormone receptor engagement), endochondromatosis (inactivating mutations in Parathyroid hormone receptor), autoimmune thyroid disease (altered function/conformation of Thyroid-stimulating hormone receptor), Grave's disease (Thyroid-stimulating hormone receptor populations studies), toxic multinodular goiter and hyperfunctioning thyroid adenomas (constitutive activation of adenylyl cyclase by Thyroid-stimulating hormone receptor), nephrogenic diabetes insipidus (decreased ligand binding or reduced expression of Arginine vasopressor receptor 2), asthma (GPR154/GPRA), partial resistance to HIV infection, reduced AIDS progression, reduced risk of Non-Hodgkin's lymphoma (altered binding affinity of Chemokine, cc motif, receptor 5), various cancers in which GPCR activity is involved, and bleeding disorder (disrupted Gᵢ/Gₒ-mediated inhibition of cAMP accumulation via Purinergic receptor, P2Y, G-protein coupled, 12). Additionally, GPR15 has been shown to control specific homing of regulatory T cells to the large intestine lamina propria, indicating that GPR15 may play a role in mucosal immune tolerance. Thus, mutant forms of GPR15 resulting in reduced or loss of function may be involved in the pathogenesis of inflammatory bowel disease. See also Hernandez et al. (2003, Nature Genetics 34:70-74), Thompson et al. (2008, Methods Mol Biol 448:109-137), and Kim et al. (2013, Science 340:1456-1459), the entire content of each of which is incorporated herein by reference.

The terms “percent similarity”, “percent identity” and “percent homology” when referring to a particular sequence are used as set forth in the University of Wisconsin GCG software program and are known in the art.

A “fragment” or “portion” of a GPCR polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. A “derivative” of a GPCR or a fragment thereof means a polypeptide modified by varying the amino acid sequence of the protein, e.g. by manipulation of the nucleic acid encoding the protein or by altering the protein itself Such derivatives of the natural amino acid sequence may involve insertion, addition, deletion or substitution of one or more amino acids, and may or may not alter the essential activity of the original GPCR.

Different “variants” of GPCRs exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acid residues are substituted with conservative or non-conservative amino acids, (b) variants in which one or more amino acids are added to the GPCR, (c) variants in which one or more amino acids include a substituent group, and (d) variants in which GPCR is fused with another peptide or polypeptide such as a fusion partner, a protein tag or other chemical moiety that may confer useful properties to the GPCR, such as, for example, an epitope for an antibody, a polyhistidine sequence, a biotin moiety and the like. Other GPCRs include variants in which amino acid residues from one species are substituted for the corresponding residue in another species, either at conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to a person having ordinary skill in the art.

To the extent such allelic variations, analogues, fragments, derivatives, mutants, and modifications, including alternative nucleic acid processing forms and alternative post-translational modification forms result in derivatives of a GPCR that retain any of the biological properties of the GPCR, they are included within the scope of this invention.

The term “functional” as used herein implies that the nucleic or amino acid sequence is functional for the recited assay or purpose.

The term “functional fragment” as used herein implies that the nucleic or amino acid sequence is a portion or subdomain of a full length polypeptide and is functional for the recited assay or purpose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism. As used herein, it refers to a synthetic expression vector, which comprises polypeptide coding sequences operably linked by the hand of man and arranged in a pattern not found in nature.

As used herein, the term “operably linked” may be used to refer to a regulatory sequence capable of mediating the expression of a coding sequence, which is placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule whereby the first and second nucleic acid sequences are co-transcribed on a single transcript, but are translated independently of each other (e.g., via an IRES or viral 2A peptide sequence).

The term “oligonucleotide,” as used herein refers to primers and probes and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers may be labeled fluorescently with 6-carboxyfluorescein (6-FAM). Alternatively primers may be labeled with 4,7,2′,7′-Tetrachloro-6-carboxyfluorescein (TET). Other alternative DNA labeling methods are known in the art and are contemplated to be within the scope of the invention.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like). “Mature protein” or “mature polypeptide” shall mean a polypeptide possessing the sequence of the polypeptide after any processing events that normally occur to the polypeptide during the course of its genesis, such as proteolytic processing from a polypeptide precursor. In designating the sequence or boundaries of a mature protein, the first amino acid of the mature protein sequence is designated as amino acid residue 1.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties to the sequence, particularly with regard to methods relating to the detection or isolation of the sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

The terms “transform”, “transfect”, and “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited by progeny cells or organisms of the recipient cell or organism. In other applications, the introduced nucleic acid may exist in the recipient cell or host organism only transiently.

A “clone” or “clonal cell population” is a population of cells derived from a single cell or common ancestor by mitosis.

A “cell line” is a clone of a primary cell or cell population that is capable of stable growth in vitro for many generations.

The compositions containing the agents or compounds identified using screening methods described herein can be administered for therapeutic purposes. In therapeutic applications, compositions are administered to a patient already suffering from a disease/disorder associated with aberrant GPCR activity in an amount sufficient to cure or at least partially arrest the symptoms of the disease/disorder and its complications. An amount adequate to accomplish this is defined as a “therapeutically effective amount or dose.” Amounts effective for this use will depend on the severity of the disease and the weight and general state of the patient.

As used herein, an “agent”, “candidate compound”, or “test compound” may be used to refer to, for example, nucleic acids (e.g., DNA and RNA), carbohydrates, lipids, including bioactive lipids, proteins, small and large peptides, amino acids, biogenic amines, peptidomimetics, ions, small molecules and other drugs. It is to be understood that screening against an agent may be used herein to refer to screening against a plurality of agents. Such agents may be provided in isolated form and then combined in a composition or the like in a suitable buffer (i.e., compatible with the screening assay) or provided as a mixture isolated/derived from a natural source, such as a tissue extract. Also encompassed herein are strategies involving overexpression of secreted proteins (secretome) or expression of the extracellular region of transmembrane proteins and use of same in screening assays.

Further to the above, a number of GPCR ligands have been identified using the “orphan receptor strategy” or “tissue-extract based approach”, wherein orphan GPCRs are exposed to tissue extracts instead of purified ligands. Positive extracts are then fractionated until the active component is isolated and characterized. See, for example, Reinscheid et al. 1995, Science 270:792-794; Civelli et al. 1998, Crit Rev Neurobiol 12:163-176; Hinuma et al. 1998, Nature 393:272-276; Kojima et al. 1999, Nature 402:656-660; as reviewed in Levoye et al. 2008, Drug Discovery Today 13:52-58, the entire content of each of which is incorporated herein by reference.

The term “control substance”, “control agent”, or “control compound” as used herein refers a molecule that is inert or has no activity relating to an ability to modulate a biological activity. With respect to the present invention, such control substances are inert with respect to an ability to modulate GPCR activity. Exemplary controls include, but are not limited to, solutions comprising physiological salt concentrations.

The term “GPCR modulatory agent” as used herein refers to an agent that is capable of modulating (e.g., increasing or decreasing) an activity attributable to a GPCR. Methods for screening/identifying such agents are presented herein below.

The term “agonist” as used herein refers to a molecule or substance that binds to or otherwise interacts with a receptor or enzyme to increase activity of that receptor or enzyme. Agonist as used herein encompasses both full agonists and partial agonists.

The term “antagonist” as used herein refers to a molecule that binds to or otherwise interacts with a receptor to block (e.g., inhibit) the activation of that receptor or enzyme by an agonist.

The term “ligand” as used herein refers to a naturally occurring or synthetic compound that binds to a protein receptor. Upon binding to a receptor, ligands generally lead to the modulation of activity of the receptor. The term is intended to encompass naturally occurring compounds, synthetic compounds and/or recombinantly produced compounds. As used herein, this term can encompass agonists, antagonists, and inverse agonists.

The term “functional interaction” as used herein refers to an interaction between a receptor and ligand that results in modulation of a cellular response. These may include changes in membrane potential, secretion, action potential generation, activation of enzymatic pathways and long term structural changes in cellular architecture or function.

The terms “G protein coupled receptors” and “GPCRs” are used interchangeably herein and include all subtypes of the opioid, muscarinic, dopamine, adrenergic, adenosine, rhodopsin, angiotensin, serotonin, thyrotropin, gonadotropin, substance K, substance P and substance R receptors, melanocortin, metabotropic glutamate receptors, or any other GPCR known to couple via G proteins. This term also includes orphan receptors that are known to couple to G proteins, but for which no specific ligand is known.

The term “GPCR activity” may be used herein to refer to the capacity for initiating downstream signaling in response to a given level of cognate ligand binding.

The term “aberrant GPCR activity” may be used herein to refer to altered capacity for initiating downstream signaling in response to a given level of cognate ligand binding relative to the wild type form of the GPCR.

The term “disease/disorder associated with aberrant GPCR activity” may be used herein to refer to a disease or disorder in which the pathogenesis can be traced to the altered capacity of a GPCR to initiate downstream signaling in response to a given level of cognate ligand binding relative to the wild-type form of the GPCR.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

All publications mentioned herein are incorporated herein by reference to disclose and described the methods and/or materials in connection with which the publications are cited.

### Aspects of the Invention

Before the present discovery and methods of use thereof are described, it is to be understood that this invention is not limited to particular assay methods, or test compounds and experimental conditions described, as such methods and compounds may vary. It is also to be understood that the terminology used herein is for the purpose of describing particular embodiments only, and is not intended to be limiting, since the scope of the present invention will be limited only the appended claims.

The available screening methods designed to identify endogenous/native ligands and non-native/novel surrogate ligands for GPCRs are largely confined to assaying signaling events downstream of GPCR engagement by such ligands. Such assays frequently measure/detect GPCR ligand engagement by measuring calcium influx. It is, however, noteworthy that calcium influx is not reliably quantitative, is subject to desensitization, and is not a universal feature of GPCR activation. The present inventors, therefore, sought to develop a more universal screening method that could be used to screen for GPCR modulators (e.g., ligands, agonists, or antagonists), irrespective of the specific downstream signaling pathways utilized by a particular GPCR. In so doing, the present inventors have developed a GPCR signaling sensor and methods of using same that overcome the aforementioned limitations of traditional GPCR ligand screening methods and therefore, can be employed for genetic and small molecule screens for GPCR modulators for the vast majority of GPCRs identified to date. See also Southern et al. (2013, J Biomol Screen DOI: 10.1177/1087057113475480; the entire content of which is incorporated herein by reference) with regard to terminology pertaining to endogenous and novel surrogate GPCR ligands.

Further to the above, the present inventors developed a quantitative sensor for Sdf1 chemokine signaling through the GPCR CXCR4. This sensor is based on the fact that binding of Sdf1 to CXCR4 induces internalization and degradation of CXCR4. The transgene for the sensor is comprised of two transcriptional units: CXCR4 fused to a red fluorescent protein (CXCR4-RFP) and a green fluorescent protein tethered to the cell membrane by a CaaX domain (memGFP). The two proteins are expressed from the same promoter and transcript; CXCR4-RFP is directly driven from the promoter and memGFP is expressed from an internal ribosomal entry site (IRES). This arrangement ensures that the amount of memGFP produced is directly proportional to the amount of CXCR4-RFP produced by a cell. Thus, memGFP serves two purposes: first, it marks cell membranes, allowing for easy visualization of the cells of interest in live or fixed preparations; and second, it serves as an internal reference against which levels of CXCR4-RFP on the cell membrane can be measured. Therefore, the ratio of red to green fluorescence on the cell membrane linearly reports CXCR4 signaling levels.

Accordingly, it is envisioned that the CXCR4 signaling sensor can be used in cell culture based screens to identify small molecules that activate or inactivate CXCR4 signaling. The principle on which the CXCR4 signaling sensor was developed can, furthermore, be adapted to generate a signaling sensor for essentially any GPCR. As described herein, cell culture based screens utilizing such GPCR sensors may be used to identify agents (e.g., small molecules) that can activate or inactivate GPCR signaling. With respect to orphan GPCRs (GPCRs for which natural ligands have not as yet been identified), GPCR signaling sensors and methods of using same may also be used to identify potential ligands.

With regard to the expression vectors and cassettes described herein, inclusion of the second ORF driving the expression of a membrane-tethered fluorescent protein (as mediated by the presence of an IRES) or multiprotein expression from a single ORF (as mediated by a viral 2A peptide sequence) provides at least three advantages. First, it provides an internal reference for the expression levels of the GPCR fusion protein by providing a second, different fluorescent protein, since the IRES or viral 2A peptide sequence provides expression of the second membrane-tethered fluorescent protein at stochiometric ratios to the GPCR fusion protein. This is important because theoretical considerations for reversible ligand-receptor binding and measurements presented herein show that the unbound ligand concentration is independent of the total amount of the receptor if one measures GPCR-FP/memFP ratios. See, for example, FIG. S3 and the description thereof. This is noteworthy because it demonstrates that differences in promoter activity/GPCR-FP expression levels are irrelevant to the method. In other words, the presence of the internal reference enables one to differentiate between a bona fide change in GPCR signaling and a fluctuation due to altered GPCR-FP expression from the promoter driving its expression. Second, the GPCR-FP/memFP ratio is a quantitative and linear measurement of the unbound ligand concentration. Third, the memFP allows for automated membrane thresholding, which is important to distinguish the cytoplasmic membrane bound GPCR-FP pool from the internalized GPCR-FP pool.

Support for the above described features conferred by the instant expression vectors or cassettes is presented in FIG. S3. Briefly, in order to test the effect of the levels of expression of the Sdf1-signaling sensor on the ratio of FmemRed/FmemGreen, the present inventors measured the ratios across primordia in embryos expressing different levels of the sensor. The results revealed that altering the levels of expression of the signaling sensor (FIG. S3D) does not change the ratios across the primordium (FIG. S3C). This is consistent with the theoretical equations for reversible ligand-receptor binding (FIGS. S3A and B).

Importantly, quantification GPCR-FP fluorescence in the absence of an internal reference (i.e., mem-FP) yields measurements that are not related to the free ligand concentration and do not allow for quantification of GPCR signaling.

Accordingly, expression vectors are envisioned comprising an isolated nucleic acid sequence that encodes a GPCR comprising, for example, SEQ ID NO: 2 or a functional fragment thereof, wherein the nucleic acid sequence encoding the GPCR is arranged in tandem with a nucleic acid sequence that encodes a first detectable signal (e.g., RFP), such that the GPCR is expressed as a fusion protein with the first detectable signal; and an isolated nucleic acid sequence that encodes a second detectable signal, wherein the isolated nucleic acid sequence that encodes a second detectable signal is arranged in tandem with a nucleic acid sequence that encodes a membrane localizing signal; wherein the GPCR fusion protein and membrane localized second detectable signal are expressed by the same promoter and nucleic acid sequences encoding each of which are linked by an internal ribosomal entry sequence (IRES) or a viral A2 peptide sequence. In an exemplary tandem arrangement, nucleic acid sequences encoding the GPCR fusion protein are positioned 5′ to nucleic acid sequences encoding the membrane localized second detectable signal. Cells comprising these expression vectors are also envisioned, as are screening assays utilizing same.

In another aspect, expression vectors are envisioned comprising an isolated GPCR-encoding nucleic acid sequence comprising, for example, SEQ ID NO: 1 or a functional fragment thereof, arranged in tandem with a first detectable signal-encoding nucleic acid sequence, wherein the GPCR-encoding nucleic acid sequence and the first detectable signal-encoding nucleic acid sequence form a single open reading frame and thus encode a fusion protein comprising the GPCR and the first detectable signal; and an isolated a second detectable signal-encoding nucleic acid sequence, wherein the isolated nucleic acid sequence that encodes a second detectable signal is arranged in tandem as a single open reading frame with a membrane localizing signal-encoding nucleic acid sequence; wherein the expression construct comprises a single promoter which drives expression of the GPCR fusion protein and the membrane localized second detectable signal and nucleic acid sequences encoding each of which are linked by an internal ribosomal entry sequence (IRES). In an exemplary tandem arrangement, nucleic acid sequences encoding the GPCR fusion protein are positioned 5′ to nucleic acid sequences encoding the membrane localized second detectable signal. Cells comprising these expression vectors are also envisioned, as are screening assays utilizing same.

In another embodiment, the expression construct comprises a single promoter which drives expression of the GPCR fusion protein and the membrane localized second detectable signal and nucleic acid sequences encoding each of which are linked by a viral A2 peptide sequence. Viral Al peptide sequence, for example, induces ribosomal skipping such that the peptide bond between two consecutive amino acids is not formed. This results in the translation of two peptides from one transcriptional unit.

Exemplary nucleic and amino acid sequences GPCRs are known in the art and accessible via a various information depositories, including Online MIM and GENBANK®, the National Institutes of Health genetic sequence database, which is an annotated collection of all publicly available sequences.

### Preparation of GPCR-Encoding Nucleic Acid Molecules and GPCR Polypeptides

Nucleic Acid Molecules: Nucleic acid molecules encoding polypeptides described herein, such as GPCR fusion proteins may be prepared by two general methods: (1) Synthesis from appropriate nucleotide triphosphates; or (2) Isolation from biological sources. Both methods utilize protocols well known in the art.

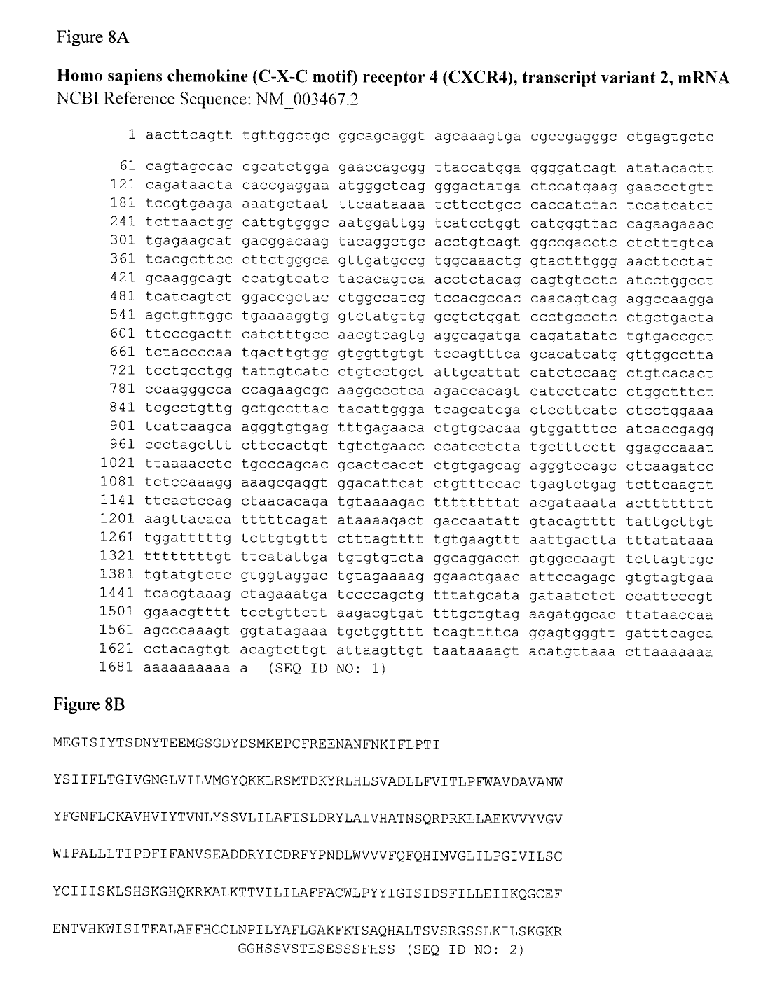

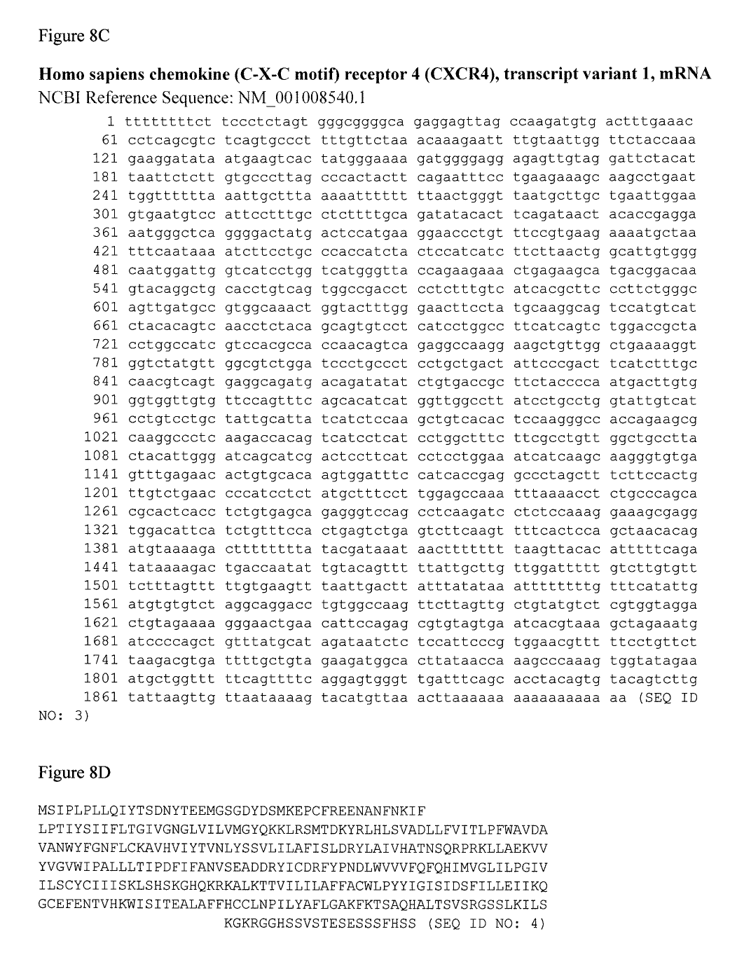

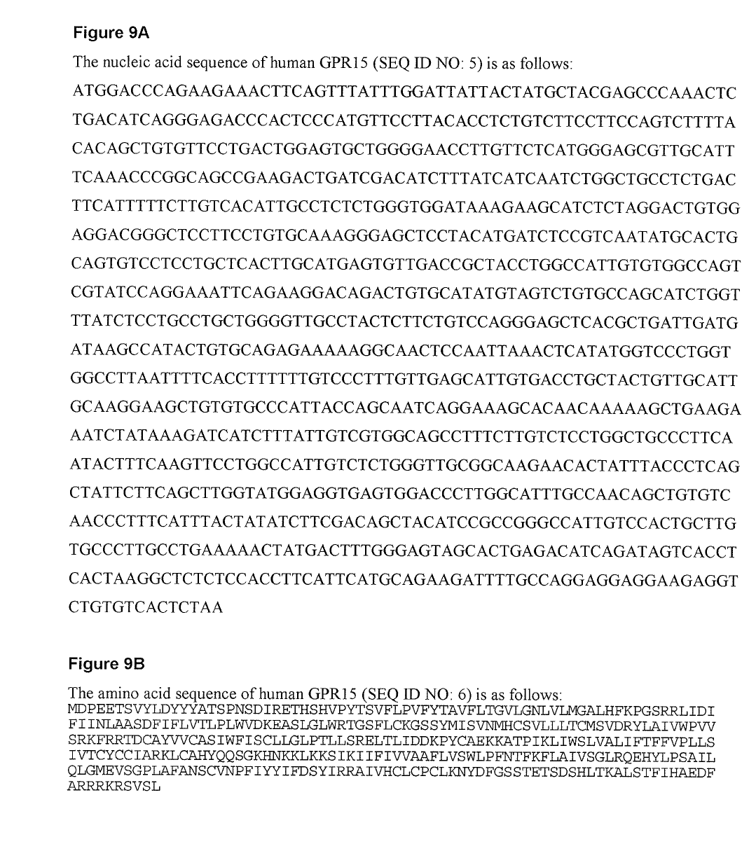

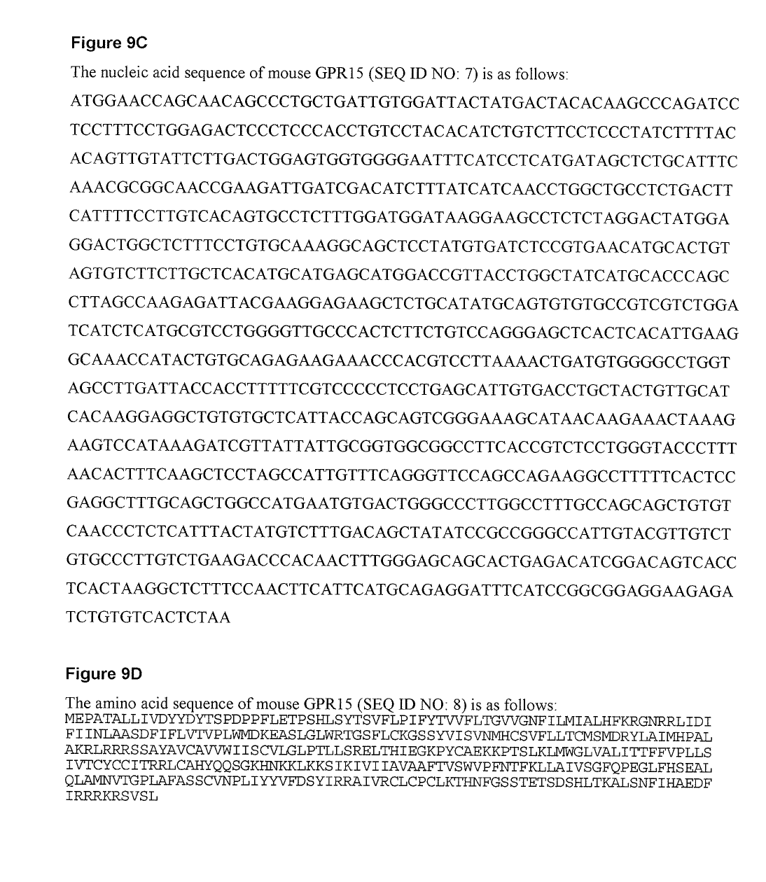

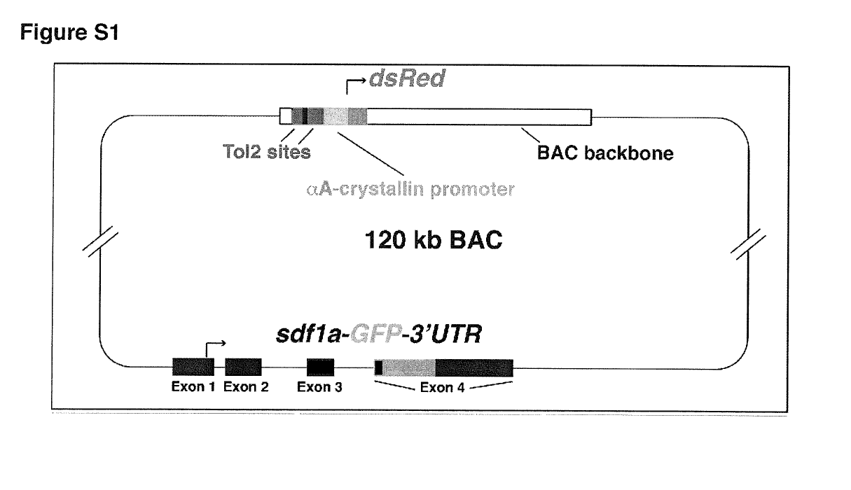

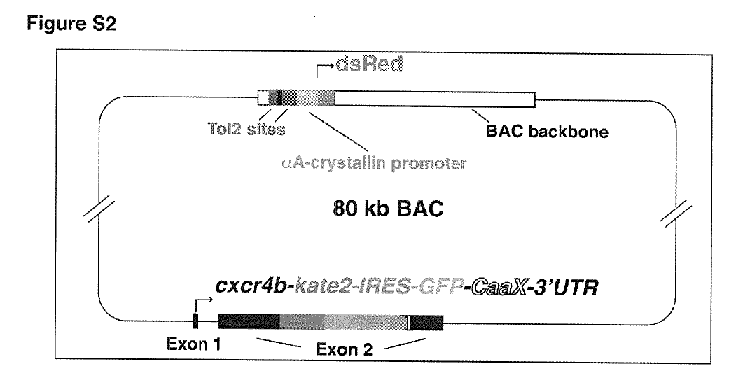

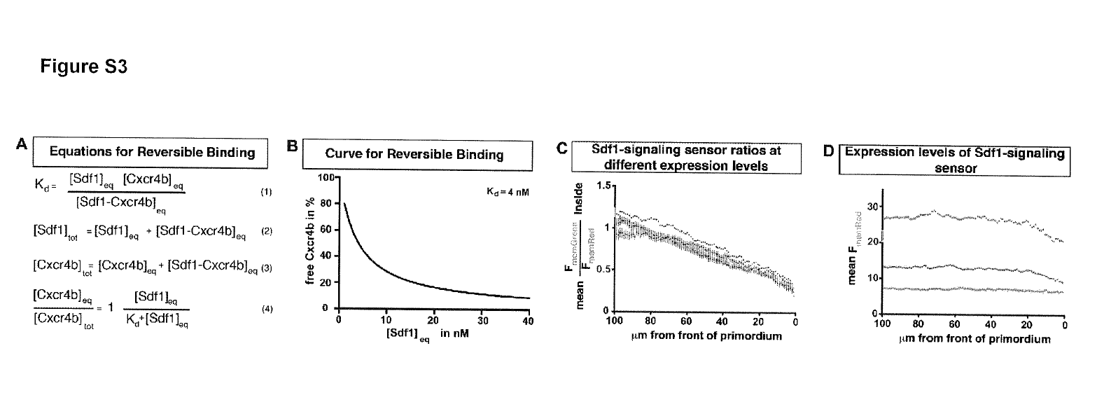

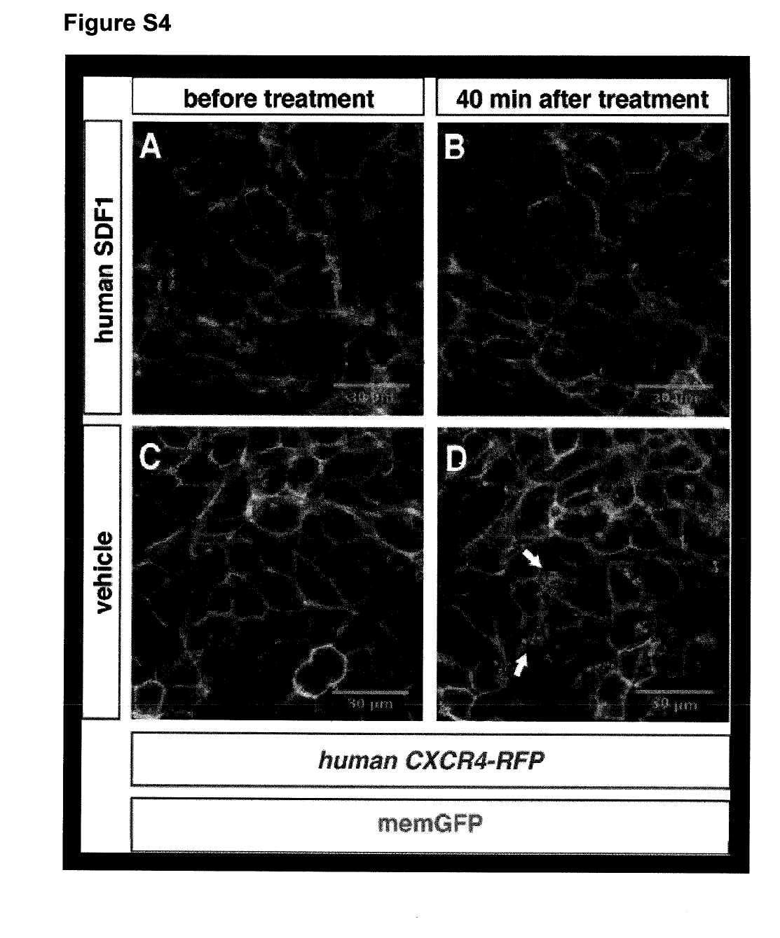

The availability of nucleotide sequence information, such as a full length cDNA of SEQ ID NO: 1 (See FIG. 8), enables preparation of an isolated nucleic acid molecule of the invention by oligonucleotide synthesis. Synthetic oligonucleotides may be prepared by the phosphoramidite method employed in the Applied Biosystems 380A DNA Synthesizer or similar devices. The resultant construct may be purified according to methods known in the art, such as high performance liquid chromatography (HPLC). Long, double-stranded polynucleotides, such as a DNA molecule of the present invention, must be synthesized in stages, due to the size limitations inherent in current oligonucleotide synthetic methods. Synthetic DNA molecules constructed by such means may then be cloned and amplified in an appropriate vector. Nucleic acid sequences encoding a GPCR may be isolated from appropriate biological sources using methods known in the art. In a preferred embodiment, a cDNA clone is isolated from a cDNA expression library of bacterial origin. In an alternative embodiment, utilizing the sequence information provided by the cDNA sequence, genomic clones encoding a GPCR may be isolated.

In accordance with the present invention, nucleic acids having the appropriate level of sequence homology with the protein coding region of SEQ ID NO: 1 may be identified by using hybridization and washing conditions of appropriate stringency. For example, hybridizations may be performed using a hybridization solution comprising: 5×SSC, 5× Denhardt's reagent, 0.5-1.0% SDS, 100 micrograms/ml denatured, fragmented salmon sperm DNA, 0.05% sodium pyrophosphate and up to 50% formamide. Hybridization is generally performed at 37-42° C. for at least six hours. Following hybridization, filters are washed as follows: (1) 5 minutes at room temperature in 2×SSC and 0.5-1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is (Sambrook et al., 1989): Tₘ=81.5° C. 16.6 Log [Na+]+0.41(% G+C)−0.63 (% formamide)−600/#bp in duplex. As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

As can be seen from the above, the stringency of the hybridization and wash depend primarily on the salt concentration and temperature of the solutions. In general, to maximize the rate of annealing of the two nucleic acid molecules, the hybridization is usually carried out at 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 micrograms/ml denatured salmon sperm DNA at 42° C. and wash in 2×SSC and 0.5% SDS at 55° C. for 15 minutes. A high stringency hybridization is defined as hybridization in 6×SSC, 5× Denhardt's solution, 0.5% SDS and 100 micrograms/ml denatured salmon sperm DNA at 42° C. and wash in 1×SSC and 0.5% SDS at 65° C. for 15 minutes. A very high stringency hybridization is defined as hybridization in 6×SSC, 5× Denhardt's solution, 0.5% SDS and 100 micrograms/ml denatured salmon sperm DNA at 42° C. and wash in 0.1×SSC and 0.5% SDS at 65° C. for 15 minutes.

Nucleic acids described herein may be maintained as DNA in any convenient cloning vector. In a preferred embodiment, clones are maintained in a plasmid cloning/expression vector, such as pBluescript (Stratagene, La Jolla, Calif.), which is propagated in a suitable E. coli host cell. Genomic clones of the invention encoding a GPCR gene may be maintained in lambda phage FIX II (Stratagene).

GPCR-encoding nucleic acid molecules of the invention include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule described herein, such as selected segments of a cDNA of SEQ ID NO: 1. Such oligonucleotides are useful, for example, as probes for detecting GPCR expression levels.

It will be appreciated by persons skilled in the art that variants (e.g., allelic variants) of GPCR sequences exist within species, and must be taken into account when designing and/or utilizing oligonucleotides of the invention. Accordingly, it is within the scope of the present invention to encompass such variants, with respect to GPCR sequences disclosed herein or the oligonucleotides targeted to specific locations on the respective genes or RNA transcripts. With respect to the inclusion of such variants, the term “natural allelic variants” is used herein to refer to various specific nucleotide sequences and variants thereof that would occur in a given DNA population. Genetic polymorphisms giving rise to conservative or neutral amino acid substitutions in the encoded protein are examples of such variants. Additionally, the term “substantially complementary” refers to oligonucleotide sequences that may not be perfectly matched to a target sequence, but the mismatches do not materially affect the ability of the oligonucleotide to hybridize with its target sequence under the conditions described.

Thus, the coding sequence may be that shown in, for example, SEQ ID NO: 1, or it may be a mutant, variant, derivative or allele of this sequence. The sequence may differ from that shown by a change which is one or more of addition, insertion, deletion and substitution of one or more nucleotides of the sequence shown. Changes to a nucleotide sequence may result in an amino acid change at the protein level, or not, as determined by the genetic code.

Thus, nucleic acid according to the present invention may include a sequence different from the sequence shown in SEQ ID NO: 1, but which encodes a polypeptide with the same amino acid sequence.

On the other hand, the encoded polypeptide may comprise an amino acid sequence which differs by one or more amino acid residues from the amino acid sequence shown in SEQ ID NO: 2. See FIG. 8. Nucleic acid encoding a polypeptide which is an amino acid sequence mutant, variant, derivative or allele of the sequence shown in SEQ ID NO: 2 is further provided by the present invention. Nucleic acid encoding such a polypeptide may show greater than 60% identity with the coding sequence shown in SEQ ID NO: 1, greater than about 70% identity, greater than about 80% identity, greater than about 90% identity or greater than about 95% identity.

The present invention provides a method of obtaining a nucleic acid of interest, the method including hybridization of a probe having part or all of the sequence shown in SEQ ID NO: 1, or a complementary sequence thereto, to target the nucleic acid. Successful hybridization leads to isolation of nucleic acid which has hybridized to the probe, which may involve one or more steps of polymerase chain reaction (PCR) amplification.

In certain embodiments, oligonucleotides described herein are fragments of the sequences shown in SEQ ID NO: 1, or any allele associated with GPCR activity, are at least about 10 nucleotides in length, more particularly at least 15 nucleotides in length, more particularly at least about 20 nucleotides in length. Fragments and other oligonucleotides may be used as primers or probes as discussed but may also be generated (e.g. by PCR) in methods concerned with determining the presence in a test sample of a sequence encoding a homolog or ortholog of a GPCR.

### Polypeptides:

A full-length GPCR protein of the present invention may be prepared in a variety of ways, according to known methods. The protein may be purified from appropriate sources. This is not, however, a preferred method due to the low amount of protein likely to be present in a given cell type at any time. The availability of nucleic acid molecules encoding GPCRs enables production of this protein using in vitro expression methods known in the art. For example, a cDNA or gene may be cloned into an appropriate in vitro transcription vector, such as pSP64 or pSP65 for in vitro transcription, followed by cell-free translation in a suitable cell-free translation system, such as wheat germ or rabbit reticulocyte lysates. In vitro transcription and translation systems are commercially available, e.g., from Promega Biotech, Madison, Wis. or BRL, Rockville, Md.

Alternatively, according to a preferred embodiment, larger quantities of a GPCR may be produced by expression in a suitable prokaryotic or eukaryotic system. For example, part or all of a DNA molecule, such as a cDNA of SEQ ID NO: 1, may be inserted into a plasmid vector adapted for expression in a bacterial cell, such as E. coli. Such vectors comprise regulatory elements necessary for expression of the DNA in a host cell (e.g. E. coli) positioned in such a manner as to permit expression of the DNA in the host cell. Such regulatory elements required for expression include promoter sequences, transcription initiation sequences and, optionally, enhancer sequences.

Polypeptides which are amino acid sequence variants, alleles, derivatives or mutants are also encompassed herein. A polypeptide which is a variant, allele, derivative, or mutant may have an amino acid sequence that differs from that given in a wildtype GPCR protein (e.g., SEQ ID NO: 2) by one or more of addition, substitution, deletion and insertion of one or more amino acids. Preferred such polypeptides retain GPCR function, that is to say have one or more of the following properties: an ability to trigger downstream signaling pathways in a manner consistent with the wildtype GPCR; immunological cross-reactivity with an antibody reactive with the wildtype GPCR polypeptide; and sharing an epitope with the wildtype GPCR polypeptide (as determined for example by immunological cross-reactivity between the two polypeptides.

A polypeptide which is an amino acid sequence variant, allele, derivative or mutant of the amino acid sequence shown in, for example, SEQ ID NO: 2 may comprise an amino acid sequence which shares greater than about 35% sequence identity with the sequence shown, greater than about 40%, greater than about 50%, greater than about 60%, greater than about 70%, greater than about 80%, greater than about 90% or greater than about 95%. Particular amino acid sequence variants may differ from that shown in SEQ ID NO: 2 by insertion, addition, substitution or deletion of 1 amino acid, 2, 3, 4, 5-10, 10-20, 20-30, 30-40, 40-50, 50-100, 100-150, or more than 150 amino acids. For amino acid “homology”, this may be understood to be identity or similarity (according to the established principles of amino acid similarity, e.g., as determined using the algorithm GAP (Genetics Computer Group, Madison, Wis.). GAP uses the Needleman and Wunsch algorithm to align two complete sequences that maximizes the number of matches and minimizes the number of gaps. Generally, the default parameters are used, with a gap creation penalty=12 and gap extension penalty=4. Use of GAP may be preferred but other algorithms may be used including without limitation, BLAST (Altschul et al. (1990 J. Mol. Biol. 215:405-410); FASTA (Pearson and Lipman (1998) PNAS USA 85:2444-2448) or the Smith Waterman algorithm (Smith and Waterman (1981) J. Mol. Biol. 147:195-197) generally employing default parameters. Use of either of the terms “homology” and “homologous” herein does not necessarily imply any evolutionary relationship between the compared sequences. The terms are used similarly to the phrase “homologous recombination”, i.e., the terms merely require that the two nucleotide sequences are sufficiently similar to recombine under appropriate conditions.

Insertion of the DNAs encoding GPCR polypeptide or a fragment thereof into a vector is easily accomplished when the termini of both the DNAs and the vector comprise compatible restriction sites. It may, however, be necessary to modify the termini of the DNAs and/or vector to achieve compatibility. This can be achieved by a variety of methods, including digesting single-stranded DNA overhangs generated by restriction endonuclease cleavage to produce blunt ends or filling in single-stranded termini with an appropriate DNA polymerase. Such methods are a matter of routine practice.

Alternatively, desired sites may be produced, e.g., by ligating nucleotide sequences (linkers) onto the termini. Such linkers may comprise specific oligonucleotide sequences that define desired restriction sites. Restriction sites can also be generated by the use of the polymerase chain reaction (PCR). See, e.g., Saiki et al., Science 239:487 (1988). The cleaved vector and the DNA fragments may also be modified if required by homopolymeric tailing.

Recombinant expression vectors described herein are typically self-replicating DNA or RNA constructs comprising nucleic acids encoding a GPCR or a functional fragment thereof, usually operably linked to suitable genetic control elements that are capable of regulating expression of the nucleic acids in compatible host cells. Genetic control elements may include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. Expression vectors also may contain an origin of replication that allows the vector to replicate independently of the host cell.

Vectors that could be used in this invention include microbial plasmids, viruses, bacteriophage, DNA fragments capable of integration, and other vehicles that may facilitate integration of the nucleic acids into the genome of the host. Plasmids are the most commonly used form of vector but all other forms of vectors which serve an equivalent function and which are, or become, known in the art are suitable for use herein. See, e.g., Pouwels et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

Expression of nucleic acids encoding a GPCR or a functional fragment thereof can be carried out by conventional methods in either prokaryotic or eukaryotic cells. Although strains of E. coli are employed most frequently in prokaryotic systems, many other bacteria such as various strains of Pseudomonas and Bacillus are know in the art and can be used as well.

Prokaryotic expression control sequences typically use include promoters, including those derived from the B-lactamase and lactose promoter systems (Chang et al., Nature, 198:1056 (1977)), the tryptophan (tip) promoter system (Goeddel et al., Nucleic Acids Res. 8:4057 (1980)), the lambda P_{L} promoter system (Shimatake et al., Nature, 292:128 (1981)) and the tac promoter (De Boer et al., Proc. Natl. Acad. Sci. USA 292:128 (1983)). Numerous expression vectors containing such control sequences are known in the art and available commercially.

Suitable host cells for expressing nucleic acids encoding a GPCR or a functional fragment thereof include prokaryotes and higher eukaryotes. Prokaryotes include both gram negative and positive organisms, e.g., E. coli and B. subtilis. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a wide variety of vectors for many different species. As used herein, E. coli and its vectors will be used generically to include equivalent vectors used in other prokaryotes. A representative vector for amplifying DNA is pBR322 or many of its derivatives. Vectors that can be used to express a GPCR or a functional fragment thereof include but are not limited to those containing the lac promoter (pUC-series); trp promoter (pBR322-trp); Ipp promoter (the pIN-series); lambda-pP or pR promoters (pOTS); or hybrid promoters such as ptac (pDR540). See Brosius et al., “Expression Vectors Employing Lambda-, trp-, lac-, and Ipp-derived Promoters”, in Rodriguez and Denhardt (eds.) Vectors: A Survey of Molecular Cloning Vectors and Their Uses, 1988, Buttersworth, Boston, pp. 205-236.

Higher eukaryotic tissue culture cells are preferred hosts for the recombinant production of a GPCR or a functional fragment thereof. Although any higher eukaryotic tissue culture cell line might be used, including insect baculovirus expression systems, mammalian cells are preferred. Transformation or transfection and propagation of such cells have become a routine procedure. Examples of useful cell lines include HeLa cells, HEK293 cells (human embryonic kidney 293 cell line), Chinese hamster ovary (CHO) cell lines, baby rat kidney (BRK) cell lines, insect cell lines (such as S2 cells), bird cell lines, and monkey (COS) cell lines.

Expression vectors for such cell lines usually include an origin of replication, a promoter, a translation initiation site, RNA splice sites (if genomic DNA is used), a polyadenylation site, and a transcription termination site. These vectors also usually contain a selection gene or amplification gene. Suitable expression vectors may be plasmids, viruses, or retroviruses carrying promoters derived, e.g., from such sources as adenovirus, SV40, parvoviruses, vaccinia virus, or cytomegalovirus. Representative examples of suitable expression vectors include pCR®3.1, pcDNA1, pCD (Okayama et al., Mol. Cell. Biol. 5:1136 (1985)), pMClneo Poly-A (Thomas et al., Cell 51:503 (1987)), pUC19, pREP8, pSVSPORT and derivatives thereof, and baculovirus vectors such as pAC 373 or pAC 610.

The basic molecular biology techniques used to practice the methods of the invention are well known in the art, and are described for example in Sambrook et al., 1989, Molecular Cloning: A Laboratory Manual, Cold Spring Harbor Laboratory, New York; Ausubel et al., 1988, Current Protocols in Molecular Biology, John Wiley & Sons, New York; and Ausubel et al., 2002, Short Protocols in Molecular Biology, John Wiley & Sons, New York).

As described herein below, a GPCR polypeptide or a fragment thereof, and more particularly, fusion proteins thereof, may be used in screening assays for molecules which affect or modulate GPCR activity or function. Such molecules may be useful for research purposes.

### Screening Assays:

In accordance with this embodiment, cells expressing a GPCR fusion protein comprising a full length GPCR or a functional fragment thereof fused to a first detectable signal and a membrane localized second detectable signal, wherein the GPCR fusion protein and the membrane localized second detectable signal are expressed by the same promoter and on the same transcript, are contacted with a candidate compound or a control compound and the ability of the candidate compound to interact with the GPCR component of the GPCR fusion protein is determined as described herein. If desired, this assay may be used to screen a plurality (e.g. a library) of candidate compounds.

The cell, for example, can be of prokaryotic origin (e.g., E. coli) or eukaryotic origin (e.g., yeast or mammalian). Exemplary cells and cell lines that can be used in cell-based assays include, but are not limited to, U2OS osteosarcoma cells, yeast, Xenopus melanophores, S2 cells, HEK 293 cells, COS cells, CHO cells, HeLa cells, mouse tail fibroblasts (129TF), mouse embryonic fibroblasts (MEF), mouse myoblasts (C2C12), rat mesenchymal stem cells (MSC), human fibroblasts (MRC5), human fibrosarcoma cells (HT1080), human embryonic kidney cells (293T), and rhesus macaque mammary tumor cells (CMMT). See also Qin et al. (2010, PLoS ONE 5:e10611, the entire content of which is incorporated herein by reference.

In a particular embodiment, stably expressing cell lines are envisioned for use in the screening methods described herein. Typically, stably expressing cell lines exhibit less noise and thus may have greater sensitivity. In a particular embodiment, lentiviral or retroviral transduction or the flip in system are utilized to generate stably expressing cell lines for use in screening methods described herein. An exemplary stably expressing cell line generated using the flip in system is depicted in FIG. S4, results pertaining to which are described below.

As described in the Life Technologies website, the Flp-In™ System allows stable integration and expression of a gene of interest to deliver single-copy isogenic cell lines. Flp-In™ expression involves introduction of a Flp Recombination Target (FRT) site into the genome of the mammalian cell line of choice. An expression vector containing the gene of interest is then integrated into the genome via Flp recombinase—mediated DNA recombination at the FRT site. A selection of Flp-In™ products, including expression vectors and systems, as well as parental cell lines with a stably integrated FRT site are available from Life Technologies. These products are designed for rapid generation of stable cell lines and ensure high-level expression of a protein of interest from a Flp-In™ expression vector. See also the Invitrogen website directed to Protein-Expression-and-Analysis/Protein-Expression/Mammalian-Expression/flip-in-and-jump-in-systems/flp-in-system for additional details.

In order to test whether the human version of the Sdf1-signaling sensor responds to human SDF1 in a similar fashion to that of the fish version of the Sdf1-signaling sensor, the present inventors generated a Flp-In™ T-REx™ 293 cell line that expresses human CXCR4 fused to the RFP variant Kate2 followed by an IRES and a membrane-tethered GFP from a tetracycline-inducible promoter. Because the flippin technology allows one to generate a single copy transgene in a specific genomic location of HEK 293 cells, expression levels from the human Sdf1-signaling sensor are fairly uniform (FIGS. S4A and C). Treating the Flp-In™ T-REx™ 293 cell line carrying the human Sdf1-signaling sensor with recombinant human SDF1 induces internalization of CXCR4-Kate2 within 40 minutes (FIG. S4D) but not in vehicle treated controls (FIG. S4B). This observation indicates that the human Sdf1-signaling sensor responds to human SDF1 and is suitable for screening for CXCR4 agonists, antagonists and molecular factors regulating CXCR4 internalization using chemical libraries and shRNA libraries, respectively.

In embodiments wherein the first and second detectable signals are each fluorescent proteins (such as, for example GFP and RFP), the pair of first and second detectable signals are chosen based on their distinct fluorescent emissions upon excitation with an appropriate wavelength of light. In a particular embodiment, the first detectable signal is GFP and thus, the GPCR fusion protein comprises GFP and the second detectable signal is RFP, which is localized to the membrane via a membrane localization signal. In the absence of ligand engagement and/or high levels of constitutive activity, most of the fluorescent signal emitted by each of the GPCR fusion protein and the membrane localized RFP co-localizes at the membrane and can thus be detected as co-localized immunofluorescence. In this particular embodiment, the GFP exhibits bright green fluorescence when exposed to light in the blue to ultraviolet range and the RFP exhibits a red fluorescence when exposed to light of ˜558 nm and co-localization of the two signals in merged images appears yellow. Upon ligand engagement, however, the GPCR fusion protein (comprising GFP) is internalized and this change in cellular localization can be detected by tracking the GFP emissions intracellularly and by measuring co-immunofluorescence of GPCR-GFP fusion protein and RFP in cells contacted with ligand relative to controls.

In a particular embodiment, the screening assay is performed in a tissue culture dish, and more particularly in multi-well plates (e.g., a 96-well or 384-well plate). Methods for performing such GPCR screening assays in the context of 384-well plates are described by Ross et al. 2008, J Biomolecular Screening 13:449-455, the entire content of which is incorporated herein by reference. It is to be understood that cells comprising the expression vectors described herein that express a GPCR fusion protein comprising a GPCR or functional fragment thereof fused to a first detectable signal and a membrane localized second detectable signal may be selected for expression of the first or second detectable signal and/or a different selectable marker introduced into the cells via recombineering in advance of plating in the aforementioned multi-well plates. Such selection processes may involve fluorescence activated cell sorting (FACS) in embodiments wherein the cells are recombineered to express fluorescent proteins, such as, e.g., RFP or GFP.

In accordance with the above, cells comprising the expression vectors described herein that express a GPCR fusion protein comprising a GPCR or functional fragment thereof fused to a first detectable signal (e.g., GFP) and a membrane localized second detectable signal (e.g., RFP) are contacted with a plurality of candidate ligands, which may comprise, e.g., small molecules, peptides, proteins, lipids or libraries thereof, or tissue extracts, to determine if contact with any of the candidate ligands causes a change in membrane localization of the GPCR fusion protein (i.e., internalization of the GPCR fusion protein) as detected by a decrease in co-localization of the first and second detectable signals. It is to be understood that the initial screening steps may be performed using pools of candidate ligands and that following identification of positive pools (with whom contact results in a decrease in co-localization of the first and second detectable signals), subsequent rounds of screening against sub-pools thereof and/or eventually single compounds serves to identify a single compound as a bona fide GPCR ligand or agonist.

In an alternative embodiment, screening assays described herein may be used to identify antagonists of GPCR activity. In accordance with this objective, the screening assay may include a known ligand or agonist of the GPCR or assay conditions can be established and maintained wherein a low level basal GPCR signaling activity is detectable (i.e., wherein some GPCR fusion protein internalization is detected that exceeds control levels) and candidate antagonists or pools thereof can be screened to determine if contact with a particular candidate antagonist or pool of candidate antagonists reduces internalization of the GPCR fusion protein, as detected by a increase in co-localization of the first and second detectable signals.

In a particular embodiment, the primary screens described herein are performed using 1-100 μM concentration of candidate compounds. In more particular embodiments, primary screens are performed using 1-50 μM, 1-25 μM, 1-15 μM, 10-20 μM, or 12 μM concentration of candidate compounds. See also Ross et al. (2008) J Biomolecular Screening 13:449-455, the entire content of which is incorporated herein by reference.

The screening assay may be performed by scanning live cells in multi-well plates using high throughput techniques.

In an alternative embodiment, the screening assay may be performed using gated FACS to sort recombinant cells that exhibit differential localization of GPCR fusion protein and membrane localized second detectable signal in the presence of a potential ligand, agonist, or antagonist. Such an approach would be applicable under circumstances wherein the GPCR is targeted for degradation after ligand stimulation

A number of review articles describe model systems for the identification of GPCR ligands, agonists, and antagonists, including Wise et al. Annu Rev Pharmacol Toxicol 2004, 44:43-66; Civelli et al. Pharmacology & Therapeutics 2006, 110:525-532; Frang et al. Assay and Drug Development Technologies 2003, 1:275-280; Cacace et al. Drug Discovery Today 2003, 8:785-792; Levoye et al. Drug Discovery Today 2008, 13:52-58; Ross et al. 2008, J Biomolecular Screening 13:449-455; Schneider et al. Methods in Enzymology 2010, 485:459-480; the entire content of each of which is incorporated herein. Also encompassed herein are U.S. Pat. Nos. 8,426,138; 7,927,821; and 7,872,101, the content of each of which is incorporated herein by reference in its entirety.

Once identified in a primary screen, a GPCR ligand, agonist, or antagonist may be further characterized against other GPCRs to evaluate its activity and selectivity profile prior to secondary studies using additional cell-based assays, tissue-based assays, and potentially whole animal-based assays designed to study the physiological role of the GPCR. Accordingly, methods for modulating the activity of the GPCR in question comprising contacting a cell that expresses the GPCR with a composition comprising a modulator (e.g., a ligand, agonist, or antagonist) of the GPCR identified in a primary screening assay are also envisioned. Such cells may be present as single cells, tissues, or in whole animals and may express endogenous, native GPCR or may be engineered to express exogenous GPCR via recombinant methods.

Further to the above, agents identified in screening assays described herein that modulate a GPCR activity may be tested in an animal model. Examples of suitable animals include, but are not limited to, mice, rats, rabbits, monkeys, guinea pigs, dogs and cats. In a particular embodiment, the animal is an animal model system for a human disease or disorder associated with aberrant GPCR activity. In accordance with this embodiment, the test compound or a control compound is administered (e.g., orally, rectally or parenterally such as intraperitoneally or intravenously) to a suitable animal and the effect on GPCR activity is determined.

Details pertaining to an exemplary cell-based screening assay are set forth below:
1. Cell line: Flp-In™-293 cells with pOG44 (Invitrogen)
2. Base vector: pCDNA5/FRT (Invitrogen)
3. GPCR of choice: human CXCR4
4. Transfection method: Lipofectamine® 2000 (Invitrogen). Cotransfect Flp-In™-293 cells with pOG44 (Invitrogen) and pCDNA5/FRT-CXCR4-kate-IRES-GFPF in a 9:1.
5. Selection method for stable integration: 200 ug/mL Hygromycin B.
6. Screening of stable expression lines: Assess cell lines by quantitative confocal microscopy for uniform expression.
7. Concentration of compounds for screening: 2 nM to 10 μM in log scale
8. Detection methods: automated fluorescence microscopy followed by automated image segmentation (e.g. Fero et al. Cold Spring Harb Perspect Biol 2010; doi: 10.1101/cshperspect.a000455 and Battenberg et al. Arkin Laboratory for Dynamical Genomics, Lawrence Berkeley National Laboratory, 2006; the entire content of each of which is incorporated herein by reference) if receptor is not degraded upon internalization.

### Agents Identified by the Screening Methods of the Invention

The invention provides methods for identifying agents (e.g., candidate compounds or test compounds) that modulate GPCR activity. Agents identified by the screening method of the invention are useful as candidate pharmaceutical agents for treating diseases/disorders associated with aberrant GPCR activity. Also encompassed herein is the use of such GPCR modulators for treating diseases/disorders associated with aberrant GPCR activity and the use of such GPCR modulators in the preparation of a medicament for the treatment of diseases/disorders associated with aberrant GPCR activity.

Examples of agents, candidate compounds or test compounds include, but are not limited to, nucleic acids (e.g., DNA and RNA), carbohydrates, lipids, including bioactive lipids, proteins, small and large peptides, amino acids, biogenic amines, peptidomimetics, ions, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is primarily focused on peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

Therapeutic Uses of Agents Able to Bind and/or Modulate GPCR Activity

The invention provides for treatment of disorders associated with aberrant GPCR activity by administration of a therapeutic compound identified using the above-described methods. Such compounds include, but are not limited to nucleic acids (e.g., DNA and RNA), carbohydrates, lipids, including bioactive lipids, proteins, small and large peptides, amino acids, biogenic amines, peptidomimetics, ions, antibodies, small molecules and other drugs.

Methods for treating patients afflicted with disorders associated with aberrant GPCR activity comprising administering to a subject an effective amount of a modulator (e.g., ligand, agonist, or antagonist) identified using methods described are encompassed herein. Use of such GPCR modulators for treating patients afflicted with disorders associated with aberrant GPCR activity and in the preparation of medicaments for treatment of disorders associated with aberrant GPCR activity are also envisioned. In a particular aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Also encompassed is a method for modulating the activity of a particular GPCR comprising administering to a subject having or at risk of developing a disorder associated with aberrant activity of the particular GPCR an effective amount of a composition comprising a modulator (e.g., ligand, agonist, or antagonist) of the particular GPCR.

The invention also encompasses a pharmaceutical composition comprising: (a) an agonist or antagonist identified using screening methods described herein; and (b) a pharmaceutically acceptable carrier. In one embodiment, the agonist or antagonist of the GPCR is a small molecule that binds to the GPCR. In another embodiment, the agonist or antagonist of the GPCR is an antibody or antigen-binding fragment thereof that specifically binds to the GPCR and blocks the binding of the GPCR to its ligand.

Formulations and methods of administration that can be employed when the compound comprises a nucleic acid are described herein, as are additional appropriate formulations and routes of administration.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), and construction of a nucleic acid as part of a retroviral or other vector. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as sialastic membranes, or fibers.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., 1983, Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., a target tissue or tumor, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

### Pharmaceutical Compositions

The present invention also provides pharmaceutical compositions. Such compositions comprise a therapeutically effective amount of an agent, and a pharmaceutically acceptable carrier. In a particular embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin, incorporated in its entirety by reference herein. Such compositions contain a therapeutically effective amount of the compound, preferably in purified form, together with a suitable amount of carrier so as to provide a form for proper administration to a subject. The formulation should suit the mode of administration.

In a particular embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds identified using screening methods described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of a compound identified using screening methods described herein that is effective in the treatment of a disease or disorder associated with aberrant GPCR activity (e.g., WHIM syndrome and retinitis pigmentosa, etc—see previous section) can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Suppositories generally contain active ingredient in the range of 0.5% to 10% by weight; oral formulations preferably contain 10% to 95% active ingredient. Effective doses may be extrapolated from dose-response curves derived from in vitro or animal model test systems.

### Nucleic Acids

The invention provides methods of identifying agents capable of binding and/or modulating a GPCR. Accordingly, the invention encompasses administration of a nucleic acid encoding a peptide or protein capable of modulating an activity of a GPCR, as well as antisense sequences or catalytic RNAs capable of interfering with the expression and/or activity of a GPCR (e.g., a ligand identified via screening methods described herein that activates a GPCR upon binding).

In one embodiment, a nucleic acid comprising a sequence encoding a peptide or protein capable of competitively binding to a GPCR is administered. Any suitable methods for administering a nucleic acid sequence available in the art can be used according to the present invention.

Methods for administering and expressing a nucleic acid sequence are generally known in the area of gene therapy. For general reviews of the methods of gene therapy, see Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspect, the compound comprises a nucleic acid encoding a peptide or protein capable of binding to and/or modulating an activity of a GPCR, such nucleic acid being part of an expression vector that expresses the peptide or protein in a suitable host. In particular, such a nucleic acid has a promoter operably linked to the coding region, said promoter being inducible or constitutive (and, optionally, tissue-specific). In a different embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al. (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al. (1989) Nature 342:435-438).

In a further embodiment, a retroviral vector can be used (see Miller et al. (1993) Meth. Enzymol. 217:581-599). Such retroviral vectors have been modified to delete retroviral sequences that are not necessary for packaging of the viral genome and integration into host cell DNA. The nucleic acid encoding a desired polypeptide to be used in gene therapy is cloned into the vector, which facilitates delivery of the gene into a subject. More detail about retroviral vectors can be found in Boesen et al. (1994) Biotherapy 6:291-302, which describes the use of a retroviral vector to deliver the mdr1 gene to hematopoietic stem cells in order to render the stem cells more resistant to chemotherapy. Other references illustrating the use of retroviral vectors in gene therapy are: Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses may also be used effectively in gene therapy. Adenoviruses are especially attractive vehicles for delivering genes to respiratory epithelia. Adenoviruses naturally infect respiratory epithelia where they cause mild disease. Other targets for adenovirus-based delivery systems are the liver, the central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang, et al. (1995) Gene Therapy 2:775-783. Adeno-associated virus (AAV) has also been proposed for use in gene therapy (Walsh et al. (1993) Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene and such cells are delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr (1993) Meth. Enzymol. 217:599-618; Cohen et al. (1993) Meth. Enzymol. 217:618-644; Cline (1985) Pharmac. Ther. 29:69-92) and may be used in accordance with the present invention, provided that the necessary developmental and physiological functions of the recipient cells are not disrupted. The technique provides for the stable transfer of the nucleic acid to the cell, so that the nucleic acid is expressible by the cell and preferably heritable and expressible by its cell progeny.

The resulting recombinant cells can be delivered to a subject by various methods known in the art. In a particular embodiment, epithelial cells are injected, e.g., subcutaneously. In another embodiment, recombinant skin cells may be applied as a skin graft onto the subject; recombinant blood cells (e.g., hematopoietic stem or progenitor cells) may be administered intravenously. The number of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver. In a particular embodiment, the cell used for gene therapy is autologous to the subject that is treated.

In another embodiment, the nucleic acid to be introduced for purposes of gene therapy may comprise an inducible promoter operably linked to the coding region, such that expression of the nucleic acid is controllable by adjusting the concentration of an appropriate inducer of transcription.

Direct injection of a DNA encoding a peptide or protein capable of binding to and/or modulating an activity of a GPCR may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection.

### Kits

Also encompassed herein is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

### EXAMPLES

In animals, many cells reach their destinations by migrating towards higher concentrations of an attractant. However, the nature, generation and interpretation of attractant gradients are poorly understood. Using a GFP fusion and a signaling sensor, the present inventors analyzed the distribution of the attractant chemokine Sdf1 during zebrafish posterior lateral line primordium migration, a cohort of about 200 cells that migrates over a stripe of uniform sdf1 expressing cells. The present inventors determined that a small fraction of the total Sdf1 pool is competent to signal and induces a linear Sdf1-signaling gradient across the primordium. This signaling gradient is initiated at the rear of the primordium, equilibrates across the primordium within 200 minutes and operates near steady-state. The rear of the primordium generates this gradient through continuous sequestration of Sdf1 protein by the alternate Sdf1-receptor Cxcr7. Modeling shows that this scenario is akin to a dynamic version of the classic source-sink model proposed by Francis Crick in 1970.

### Methods and Materials

Sdf1a-GFP and Sdf1-Signaling Sensor Transgenics

The Sdf1-signaling sensor and the Sdf1a-GFP fusion constructs were generated using recombineering of a bacterial artificial chromosome (BAC) spanning the cxcr4b and sdf1a genomic loci, respectively, and transgenic zebrafish were obtained by co-injecting BAC DNA and tol2 transposase mRNA into one-cell-stage embryos. A detailed description of the generation of these constructs is provided in the Extended Experimental Procedures.

Immunohistochemistry, Embryonic Manipulations and Transgenesis

In situ hybridization was conducted as previously described (Thisse and Thisse, 2008). Antibody staining was detected chromogenically with DAB or fluorescently with Cy3, Alexa488 or Alexa 647 conjugated secondary antibodies. Over-expression of sdf1a and cxcr7b was mediated by transgenes driving either gene from the zebrafish heat-shock protein 70 promoter (Halloran et al., 2000). Mosaic embryos containing either the Sdf1-signaling sensor or tg(hsp70:sdf1a) and tg(CldnB:lynlynGFP) were generated through cell transplantation at the 1000-cell to sphere stage. A detailed description of the fish strains, immunohistochemistry and mosaic analysis is provided in the Extended Experimental Procedures.

Microscopy and Image Processing

For live imaging, the primordium was imaged using a Leica SP5 II confocal laser scanning microscopy system equipped with a heated stage and HyD detectors. Time-lapse imaging of live embryos was conducted similarly using the multi-point acquisition mode in order to image multiple stage-matched embryos under identical conditions. Quantification of Sdf1a-GFP and FmemRed/FmemGreen ratios for each voxel representing part of a cell membrane in the primordium were calculated using ImageJ software. The mean FmemRed/FmemGreen ratios across the dorsal-ventral and medial-lateral axes of the primordia were plotted along the anterior-posterior axis. The slope of the Sdf1-signaling gradient across the primordium was determined by linear regression analysis of the average FmemRed/FmemGreen across the primordium. A detailed description of the imaging conditions and the imageJ-based macros for image processing is provided in the Extended Experimental Procedures.

### Supplemental Data

### Extended Experimental Procedures

Zebrafish Strains

Embryos were staged as previously described (Kimmel et al., 1995). cxcr4b^{t26035} (Knaut et al., 2003), sdf1a^{t30516} (Valentin et al., 2007) and cxcr7b^{sa16} (Kettleborough et al., 2013) homozygous mutant embryos were generated by inbreeding homozygous adults, crossing homozygous adults with heterozygous adults or inbreeding heterozygous adults. Mutant, heterozygous and wild-type embryos were distinguished through PCR-amplification of the mutated locus and sequencing or restriction digest with NlaIV (New England Biolabs) for sdf1a and HpyAV (New England Biolabs) for cxcr4b. Tg(cldnB:lynlynGFP) (Haas and Gilmour, 2006), tg(hsp70:sdf1a) (Knaut et al., 2005) and tg(hsp70:cxcr7b) (Lewellis et al., 2013) embryos were generated by crossing heterozygous transgenic adults to wild-type adults. Transgenic embryos were identified by GFP fluorescence, in situ hybridization against sdf1a and cxcr7b mRNA or by PCR-amplification of the transgene using the following primers:

tg(hsp70: sdfla) genotyping primers:
Outer PCR:
(SEQ ID NO: 61)
TGAGCATAATAACCATAAATACTA
and
(SEQ ID NO: 62)
TCTGTGGGACTGTGTTGACTGTGG
Nested PCR (using product of
outer PCR as template):
(SEQ ID NO: 63)
AGCAAATGTCCTAAATGAAT
and
(SEQ ID NO: 64)
TCTGTGGGACTGTGTTGACTGTGG
tg(hsp70: cxcr7b) genotyping primers:
Outer PCR:
(SEQ ID NO: 65)
TGAGCATAATAACCATAAATACTA
and
(SEQ ID NO: 66)
GAGGCCAATGATGAAGAGGAAGAT
Nested PCR (using product of
outer PCR as template):
(SEQ ID NO: 67)
AGCAAATGTCCTAAATGAAT
and
(SEQ ID NO: 68)
CTCTGGCTGAAGGTGCTGTG

Generation of Transgenic Strains

For the Sdf1-GFP transgene, the BAC clone CH73-199F2 was modified in two ways by recombineering. First, the Tol2 (exon 4)-FRT-GalK-FRT-Tol2 (exon 1)-alpha-Crystallin-dsRed cassette was inserted into the BAC replacing nucleotides 3008 to 3052 of the pTARBAC2 backbone using GalK as a selection marker. GalK was removed by Flippase-mediated recombination. The arms of homology were 231 bp and 242 bp fragments corresponding to nucleotides 2777 to 3007 and 3053 to 3294 of the pTARBAC2 backbone, respectively. These aims of homology were subcloned into a pBluescript vector to flank the Tol2-alpha-crystallin-dsRed targeting cassette. Second, the GFP coding sequence was inserted between the last amino acid and the stop codon of sdf1a using seamless GalK-mediated recombineering. The 51 bp and 46 bp of homology upstream and downstream of the sdf1a stop codon, respectively, were added to the GFP targeting cassette by PCR. The final BAC was characterized by restriction digest, PCR amplification and BAC-end sequencing. It was then purified with the nucleobond BAC 100 kit (Clontech) and co-injected with tol2 transposase mRNA into one-cell-stage zebrafish embryos. Stable transgenic larvae were identified by out-crossing adults injected with the transgene and by raising larvae with red fluorescence in the lens from the transgenesis marker at 5 days post fertilization. The full name of this transgenic line is tg(sdf1a:sdf1a-EGFP)p10.

For the Sdf1-signaling sensor, the BAC clone DKEY-169F10 was modified in two ways by recombineering. First, the Tol2 (exon 4)-FRT-GalK-FRT-Tol2 (exon 1)-alpha-Crystallin-dsRed cassette was inserted into the BAC replacing nucleotides 729 to 760 of its pIndigo-356 backbone using GalK as a selection marker. GalK was removed by Flippase-mediated recombination. The arms of homology were 320 bp fragments corresponding to nucleotides 409 to 728 and 761 to 1080 of the pIndigo-356 backbone, respectively. These aims of homology were subcloned into a pBluescript vector to flank the Tol2-alpha-crystallin-dsRed targeting cassette. Second, a cassette consisting of kate2, an IRES from the encephalomyocarditis virus, and EGFP-CaaX followed by FRT-kanamycin-FRT flanked by 1457 bp and 812 bp of homology upstream and downstream of the cxcr4b stop codon, respectively, was inserted between the last amino acid and the stop codon of cxcr4b using the kanamycin resistance gene as a selection marker. The kanamycin resistance gene was removed by Flippase-mediated recombination. The final BAC was characterized by restriction digest, PCR amplification and BAC-end sequencing. It was then purified with the nucleobond BAC 100 kit (Clontech) and co-injected with tol2 transposase mRNA into one-cell-stage zebrafish embryos. Stable transgenic larvae were identified by out-crossing adults injected with the transgene and by raising larvae with red fluorescence in the lens from the transgenesis marker at 5 days post fertilization. The full name of this transgenic line is tg(cxcr4b:cxcr4b-IRES-EGFP-CaaX)p7.

Fluorescent Imaging of Sdf1a-GFP

For fluorescent imaging of Sdf1a-GFP tg(sdf1a:sdf1a-GFP) embryos were fixed at 36 hpf and stained for GFP and ClaudinB (see below for details). The stained embryos were mounted on slides and z-stacks were collected with a Leica 63× oil immersion objective (NA 1.4) on a Leica SP5 II confocal microscope equipped with HyD detectors. All z-stacks were collected with identical microscope settings in the photon-counting mode. For imaging embryos over-expressing Cxcr7b, tg(hsp70:cxcr7b) embryos were raised at 28° C. to 31 hpf. At this time the embryos were heat-shocked for 1 hour at 37° C., raised at 28° C. to 36 hpf, fixed and stained. Embryos were genotyped by PCR as described above.

Quantification of Sdf1a-GFP Immunofluorescence and Sdf1a-GFP Puncta inside the Primordium

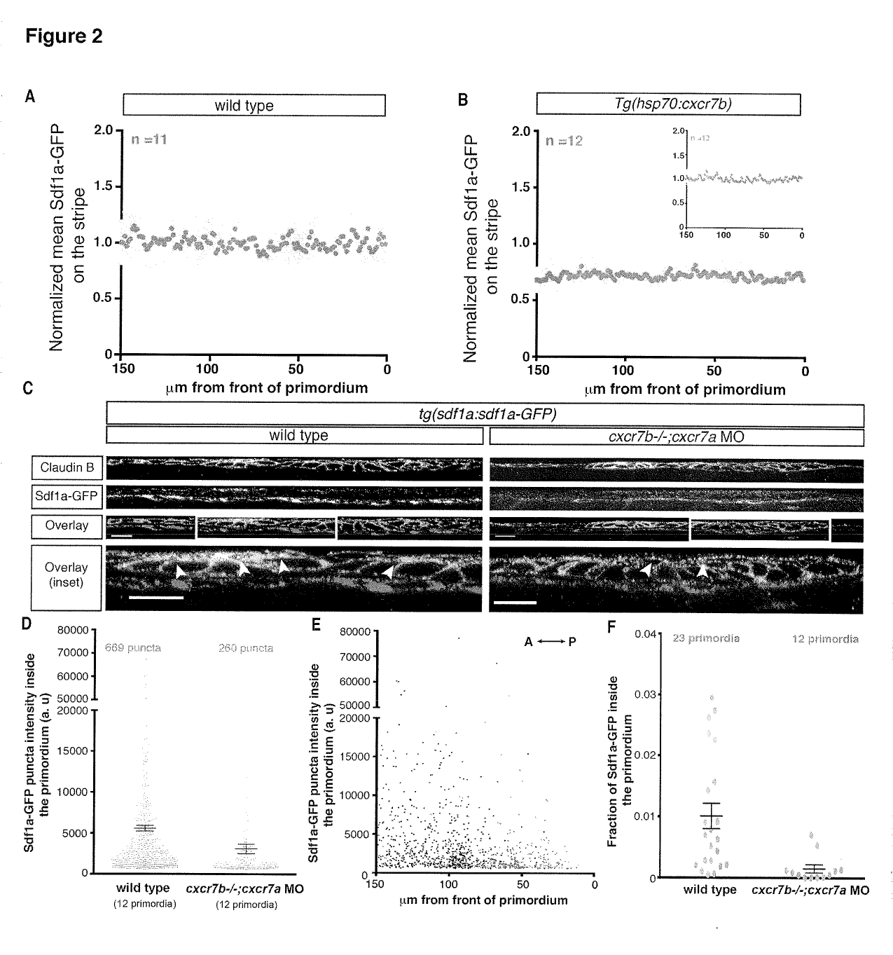

Using intensity thresholding and Gaussian blur in ImageJ (NIH), a mask was applied to the image based on the ClaudinB channel, which was refined to include only voxels within the primordium. A predefined intensity thresholding algorithm in ImageJ (NIH) was applied on the Sdf1a-GFP channel to eliminate background. For analysis of the uptake of Sdf1a by the primordium, all values of Sdf1a-GFP outside the ClaudinB mask were discarded. An intensity threshold equal to the average fluorescent intensity of Sdf1a-GFP outside of the primordium in control embryos (tg(sdf1a:sdf1a-GFP) embryos stained in the same tube) and a volume threshold of 0.1 μm³ was applied and the 3D object-counter in ImageJ (NIH), was used to count Sdf1a-GFP puncta within the primordium. A custom ImageJ (NIH) macro language script was written in order to automate this analysis. To correct for differences in staining between embryos stained in different tubes, a scaling factor was determined using the average fluorescent intensity of Sdf1a-GFP outside of the primordium in tg(sdf1a:sdf1a-GFP) control embryos and applied to all the embryos stained in a given tube. For averaging across embryos of the same genotype, the front of the primordium in each Z-stack was assigned to the 0 μm position, and the number and intensity of Sdf1a-GFP puncta within 150 μm from the front of the primordium were plotted (FIG. 2E). For analyzing the distribution of Sdf1a-GFP on the stripe, the ClaudinB mask was inverted and applied to the background corrected Sdf1a-GFP Z-stack, and Sdf1a-GFP intensities outside the mask were averaged. Sdf1a-GFP intensities in embryos of different genotypes were binned and normalized to the average Sdf1a-GFP intensities of wild-type embryos stained in the same tube. For embryos expressing cxcr7b from the heat-shock inducible promoter, Sdf1a-GFP intensities were normalized to the average Sdf1a-GFP intensities in heat-shocked, non-transgenic, wild-type siblings stained in the same tube. For computing the fraction of Sdf1a-GFP uptake inside a primordium, the total intensity of Sdf1a-GFP over a distance of 150 μm inside the primordium was divided by the sum of the intensity of Sdf1a-GFP inside and outside the primordium over the same distance.

Live Imaging Sdf1-Signaling Sensor Embryos

Live tg(cxcr4b:cxcr4b-kate2-IRES-memGFP) embryos were mounted in 0.5% low-melt agarose/Ringer's solution (HEPES 5 mM, NaCl 111 mM, KCl 5 mM, CaCl2 1 mM, MgSO4 0.6 mM). Z-stacks were collected with a Leica 40× water dipping lens (NA 0.8) and a Leica SP5 II confocal microscope equipped with HyD detectors (Leica Microsystems) and a heated stage (Warner Instruments). The temperature of the water bath was monitored and maintained between 27.9° C. and 28.4° C. All Z-stacks were collected in the photon-counting mode with identical microscope settings with the exception of the time-lapse images, which were collected using multi-point acquisition in order to image multiple embryos over the same imaging period and lower laser powers and a larger pinhole and z-step size in order to collect sufficient light while minimizing bleaching and phototoxicity. For imaging live embryos over-expressing Sdf1a, tg(hsp70:sdf1a) embryos were raised at 28° C. to 32 hpf. At this time the embryos were heat-shocked for 50 minutes at 37° C., raised at 28° C. to 36 hpf, mounted and imaged at 28° C. For imaging live embryos over-expressing Cxcr7b, tg(hsp70:cxcr7b) embryos were raised at 28° C. to 31 hpf. At this time the embryos were heat-shocked for 1 hour at 37° C., raised at 28° C. to 36 hpf, mounted and imaged at 28° C. For imaging cxcr7 deficient embryos, only primordia that did not migrate further than somite 2 by 36 hpf were imaged. Embryos were genotyped by in situ hybridization and/or PCR as described above.

Quantification of the Sdf1-Signaling Sensor

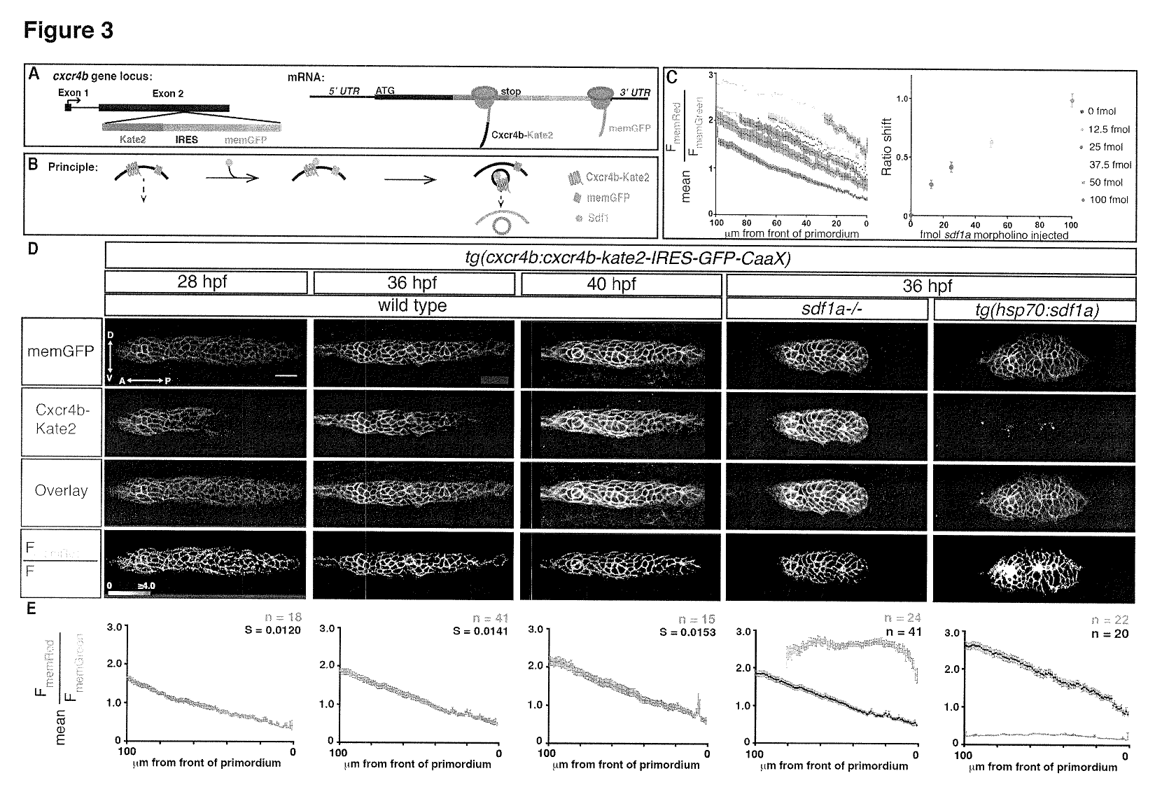

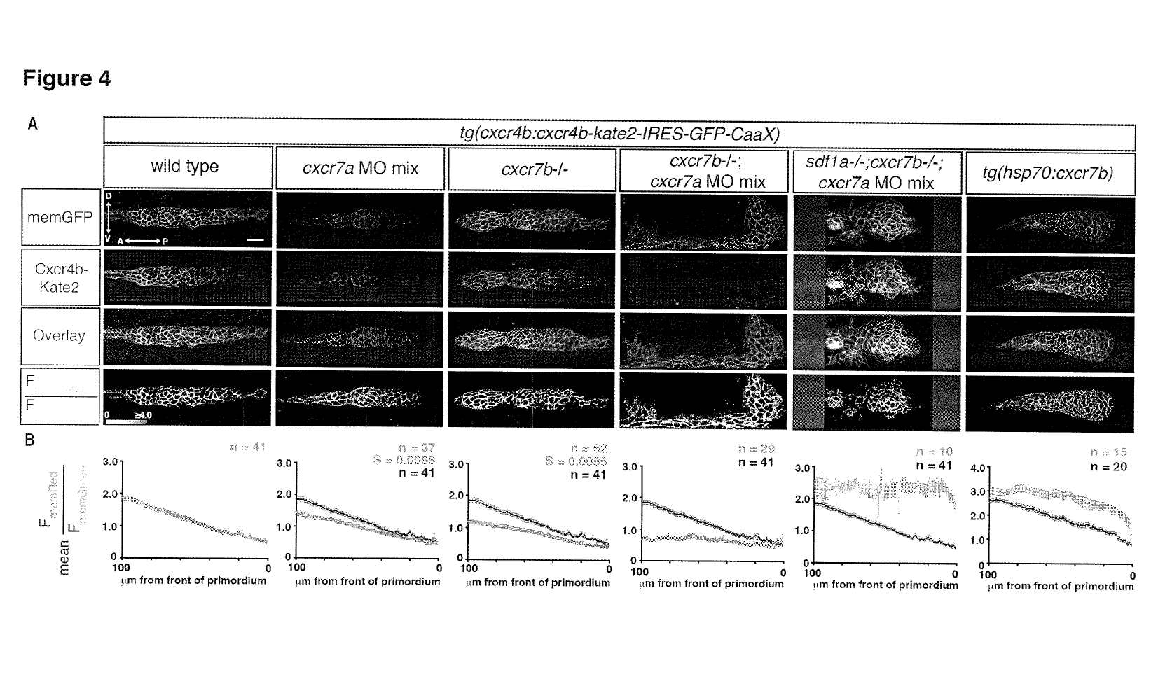

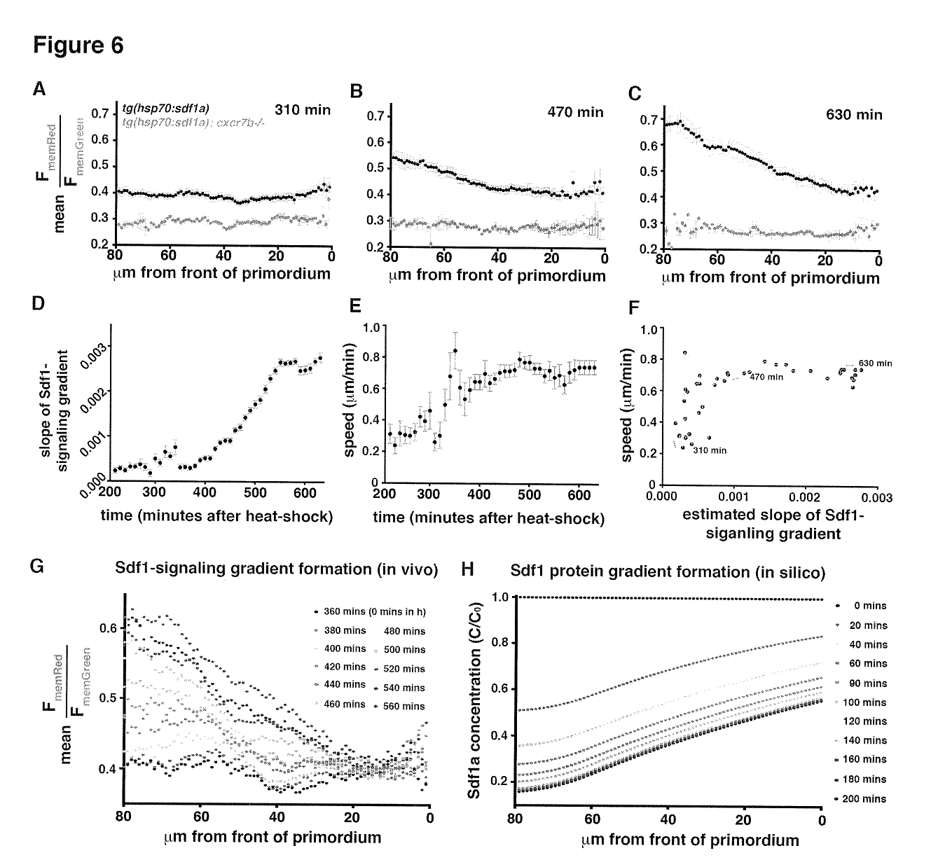

Using ImageJ (NIH), a mask was applied to the GFP channel with a threshold to selectively mark the cell membranes of the cells that comprise the primordium. All values in the GFP and Kate2 channels outside of the mask were discarded and the photon counts from the Kate2 channel were divided by the photon counts from the GFP channel for each voxel to yield a Z-stack in which each value-containing voxel represents the ratio of Kate2 fluorescence to GFP fluorescence on the cell membrane. A custom ImageJ (NIH) macro language script was written in order to automate this analysis. The data was then averaged across the dorsal-ventral axis and placed in 1-micron bins in order to yield 100 data points for still images or 80 data points for time-lapse images that correspond to the first 100 μm or the first 80 μm, respectively, from the front tip of the primordium. A shorter length was used for time-lapse images because the primordium frequently rounds up and becomes shorter than 100 μm while recovering from a global pulse of Sdf1a. For averaging across embryos with identical genotype, the front of the primordium in each Z-stack was assigned to the 0 μm position. Note: differences in overall ratios in the time-lapse experiments (e.g. FIG. 6) compared to the single time-point experiments (e.g. FIG. 3 and FIG. 4) are due to the different laser power settings that were used for each type of experiment as described in a previous section.

Linearity of the Sdf1-Signaling Sensor

Tg(cxcr4b:cxcr4b-kate2-IRES-memGFP) embryos were injected alternately with 0, 0.5, 1.0 and 1.5 nl of 0.025 mM or 0.1 mM sdf1a morpholino solution (Doitsidou et al., 2002). Alternating the injection volumes controls for possible variations in the injection rig and needle over the course of the injection. Uninjected and morphant embryos were imaged alternately at 36 hpf to control for possible variations in the microscope and embryos over the course of imaging. Image collection and gradient quantification were performed as described above. The shift of the Sdf1-signaling gradient was determined by fitting the average ratios to a second order polynomial using the least squares regression in ImageJ (NIH) and extracting the y-intercept.

Time-Lapse Imaging and Analysis

For time-lapse imaging, embryos were mounted in agarose as described in a previous section. Z-stacks were collected on a Leica SP5 II confocal microscope using a 40× water-dipping objective (NA 0.8) and multi-point acquisition to image multiple wild-type and transgenic embryos simultaneously. For FIG. 6, embryos were heat-shocked until the temperature of the embryo medium reached 37° C., at which point the embryos were returned to 28° C. for a short recovery period and then mounted. Starting several hours later, the embryos were imaged every 10 minutes over the following 7-8 hours. FIG. 6 represents one trial of this experiment in which eight wild-type and two cxcr7b−/− embryos, all carrying tg(hsp70:sdf1a), were imaged simultaneously. For determining the speed of the primordium in the tg(hsp70:sdf1a) embryos described in FIG. 6, a maximum projection of the memGFP channel was used to generate a segmented line that tracks the front tip of the primordium through time. A kymograph was then generated based on this line using the MultipleKymograph plugin written for ImageJ by J. Rietdorf and A. Seitz (see worldwide web at embl.de/eamnet/html/body_kymograph). Speeds were then attained by running the tsp040421.txt macro (same authors) on a segmented line that tracks the movement of the front tip of the primordium throughout the kymograph. The average speed of 8 primordia was then calculated for each time point.

Calculation of the Slope of the Sdf1-Signaling Gradient

The average FmemRed/FmemGreen ratios across primordia of a given genotype were calculated as discussed above and plotted against the anterior-posterior position in 1-micron bins. To determine the slope of the Sdf1-signaling gradient, the average ratios across the front 100 μm of the primordium were fitted to a linear equation using the least square regression analysis in ImageJ (NIH). For analysis of the recovery of the slope of the Sdf1-signaling gradient in FIG. 6, the same approach was used but the mean FmemRed/FmemGreen of the first 80 μm from the tip of the primordium was fitted using linear least square regression. Regression analysis was performed using Prism 6 (GraphPad).

Quantification of the Primordium Migration Defect in Different Genetic Scenarios

Wild-type and sdf1a−/− embryos were fixed in 4% paraformaldehyde (Sigma) at 48 hpf and stained for trop2 mRNA in order to visualize the primordium and determine its position relative to the somites at this stage. For determining the migration defect in the absence of cxcr7, tg(CldnB:GFP) embryos in the wild-type and cxcr7b−/− genetic backgrounds were injected with the cxcr7a morpholino mix and imaged at 48 hpf to determine the position of the primordium relative to the somites.

Calculation of the Fraction of Signaling Competent Sdf1a

To calculate the fraction of signaling competent Sdf1a from the total Sdf1a, it was assumed that the Sdf1-signaling sensor ratios in cxcr7 deficient embryos report baseline levels of signaling competent Sdf1a on the stripe and that the Sdf1-signaling sensor ratios in sdf1a−/− embryos report the absence of Sdf1a protein. Therefore, the difference in the ratios between these two scenarios corresponds to the total levels of Sdf1a protein on the stripe (C_{Sdf1a}(t=0)) in terms of Sdf1-signaling sensor ratios ((ratio_{cxcr7}−ratio_{sdf1a})=1.998). The reduction in Sdf1a protein levels is thus given as ((ratio_{sdf1a}−ratioₓ)/1.998)×C_{Sdf1a}(t=0).

Mosaic Analysis of Cxcr7 Function in Live Embryos

Wild-type tg (cxcr4b:cxcr4b-kate2-IRES-memGFP) or tg (cxcr4b:cxcr4b-kate2-IRES-memGFP); cxcr7b−/− donor embryos injected with 0.5 to 0.7 nl of cxcr7a MO-A (0.5 mM) and cxcr7a MO-B (0.5 mM) were injected with Cascade Blue Dextran (Invitrogen) as a lineage tracer at the one cell stage. At the 1000-cell to sphere stage, about 100 cxcr7 deficient donor cells were transplanted into stage-matched, wild-type tg (cxcr4b:cxcr4b-kate2-IRES-memGFP) recipient embryos. Sdf1-signaling across mosaic primoridia in live embryos was analyzed as described above, divided into signaling within the clone and the host by applying a mask to the Cascade Blue-labeled donor cells, averaged across the dorsal-ventral and medial-lateral axes and plotted along the anterior-posterior axis using ImageJ (NIH).

Morpholino Injections and Validation

Morpholino sequences (all from Gene Tools):

sdfla-MO (Doitsidou et al., 2002):
(SEQ ID NO: 69)
5′-ATCACTTTGAGATCCATGTTTGCA-3′
cxcr7a-MO-A:
(SEQ ID NO: 70)
5′-AATCCAGGUITTCGTTCTCATGCGC-3′
cxcr7a-MO-B:
(SEQ ID NO: 71)
5′-AGCTGAAGTGATCCTGTCTGCGCTT-3′

For assessment of the linearity of the Sdf1-signaling sensor, the sdf1a translation-blocking morpholino was used at the following volumes and concentrations: 0, 0.5, 1.0 and 1.5 nl and 0.025 mM and 0.100 mM.

For all experiments involving cxcr7a knockdown, two cxcr7a-MO-A and cxcr7a-MO-B were co-injected at the one-cell-stage at a concentration of 0.5 mM each and a volume of 1 nl. In order to verify specific reduction of cxcr7a mRNA translation, one-cell-stage embryos were injected with 1 nl of 50 ng/μl lynlyn-mCherry mRNA and 100 ng/μl cxcr7a-SuperFolderGFP mRNA alone or 1 nl of 50 ng/μl lynlyn-mCherry mRNA and 100 ng/μl cxcr7a-SuperFolderGFP mRNA in addition to 1 nl of cxcr7a-Mo-A and 0.5 mM of cxcr7a-Mo-B at 0.5 mM each. Embryos were mounted in 0.5% low-melt agarose/Ringer's solution (HEPES 5 mM, NaCl 111 mM, KCl 5 mM, CaCl2 1 mM, MgSO4 0.6 mM) and imaged at ˜7 hpf. Z-stacks were collected with a Leica 40× water dipping lens (NA 0.8) and a Leica SP5 II confocal microscope equipped with HyD detectors (Leica Microsystems).

The cxcr7a-sfGFP construct was generated by fusing the cxcr7a 5′-UTR and coding sequence (excluding the stop codon) to the Superfolder GFP coding sequence, separated by a two amino acid GlySer-linker. This construct was subcloned into the pCS2+ expression vector for in vitro mRNA synthesis using the mMessage mMachine kit (Ambion). The following primers were used for cloning:

cxcr7a-sense:
(SEQ ID NO: 72)
ccggagatctAGGATCACTTCAGCTCATCTGCGCATGAGAACGAAACCC
cxcr7a-sfGFP-antisense:
(SEQ ID NO: 73)
CCTTGCTCACCATgctaccAGTCACAGTCGGAGGGTTGTTC
cxcr7a-sfGFP-sense:
(SEQ ID NO: 74)
CCCTCCGACTGTGACTggtagcATGGTGAGCAAGGGCGAGG
sfGFP-antisense:
(SEQ ID NO: 75)
ccggctcgagCTACTTGTACAGCTCGTCCATGC

Global and Local Misexpression of Sdf1a

For ectopic expression of Sdf1a in clones of cells near the primordium, ˜50 cells from a 1000-cell to sphere stage tg(hsp70:sdf1a) donor embryo were transplanted into a stage-matched tg(cldbB:lynlynGFP) embryo. Embryos were raised at 28° C. until 24 hpf. Starting at this stage, embryos were heat-shocked at 39° C. for 30 minutes every 3 hours until fixation at approximately 36 hpf. Sdf1a mis-expressing cells were identified in fixed embryos by in situ hybridization against sdf1a mRNA, and the primordium was identified by antibody staining of GFP protein. For global expression of Sdf1a, tg(hsp70:sdf1a) fish were crossed to tg(cldbB:lynlynGFP) fish to obtain double transgenic embryos. These embryos were raised at 28° C. until 30 hpf. They were then heat-shocked at 39° C. from 30 to 31 hpf and from 32 to 32.5 hpf. The embryos were fixed at 32.5 hpf and stained as described above. Brightfield images were collected with on an Axioplan microscope (Zeiss) with a 10× (NA 0.5) or 40× (NA 1.3) oil-immersion objective equipped with an AxioCam camera (Zeiss).

Whole-Mount in situ Hybridization and Antibody Staining

RNA probe synthesis and in situ hybridization was performed as previously described (Thisse 2008). RNA probes against kate2, trop2, cxcr4b, sdf1a, cxcr7a and cxcr7b were labeled with DIG (Roche) and detected with anti-DIG antibody coupled to alkaline phosphatase (1:5000, Roche) and NBT/BCIP (Roche) or anti-DIG coupled to horseradish peroxidase (1:1000, Roche) and Cy3-tyramide signal amplification (Perkin Elmer). For antibody stainings, antibodies against GFP (rabbit anti-GFP, 1:500 Torrey Pines; chicken anti-GFP, Abcam, 1:1000; goat anti-GFP, 1:100, Covance), Kate2 (1:2000, Evrogen) and ClaudinB (1:2000) (Kollmar et al., 2001) were detected with anti-rabbit-Alexa488 (1:500, Invitrogen), anti-rabbit-Cy3 (1:500, Jackson ImmunoResearch), anti-chicken-Alexa488 (1:500, Invitrogen), anti-goat-Cy3 (1:500, Jackson ImmunoResearch), anti-rabbit-Alexa647 (1:500, Jackson ImmunoResearch) or anti-rabbit-HRP antibody (1:2500, Jackson ImmunoResearch) with DAB (Roche).

Generation of Flp-In™ T-REx™ Cell Lines Carrying a Single, Tetracycline-Inducible Copy of the Human Sdf1-Signaling Sensor.

Human CXCR4-Kate2-IRES-GFP-CaaX was inserted into the FRT sites in the genome of the Flp-In™ T-REx™ cell line using hygromycin selection (Invitrogen). Five different cell lines were established and cell line number 4 was used to characterize the activity of recombinant human SDF1 on the human Sdf1-signaling sensor. Expression of the human Sdf1-signaling sensor was induced using tetracycline and cells were imaged 24 hours after induction of expression using a Leica SP5 II confocal microscope.

### Supplemental Equations and Discussion

Mathematical Modeling of Chemokine Gradient Formation

### Introduction

The model addresses the question of whether it is feasible for a chemokine sink localized to the rear of the primordium to generate a stable concentration gradient for the chemokine that would be sensed by cells ahead of the sink in the primordium. This gradient would form largely by diffusion, move with the primordium and, in effect, ‘bootstrap’ the primordium into its self-generated gradient.

The present experiments and other data impose several constraints that must be met by a model. The free diffusion coefficient, D, for the chemokine, Sdf1, has been measured at about 100 μm² s⁻¹ (Veldkamp, 2005), which is consistent with a protein with molecular mass of 8 kDa. The present work shows that the velocity of the primordium, u, is 0.7 μm min⁻¹ (0.012 μm s⁻¹) and the concentration gradient forms in about 200 min. The gradient is sensed by a region of primordium that is about 100 μm in extent while the sink extends a variable distance in the rear of the primordium depending on position along the chemokine stripe.

An essential aspect of the model is that it must generate a steady-state condition. Two possible models for gradient formation may be hypothesized. In the first, the motion of the primordium itself into an essentially infinitely long stripe of chemokine is sufficient to produce a steady-state. In the second, the motion is neglected and the stationary sink in the rear of the primordium will continually remove chemokine leading to an ever-changing gradient unless continuous production and degradation are postulated. Based on the Peclet number it is shown that the first model need not be considered and the second model is developed. Extension of the second model to a moving coordinate system confirms that the moving primordium has little effect for expected values of D and u.

### Peclet Number

Many problems involve diffusion and advection where typically a source of diffusing material is present in a flowing medium and the resulting pattern of transport of the material is either affected by both processes or dominated by one. The dominance may be quantified by a dimensionless Peclet number, Pe, that compares the ratio of the characteristic times for diffusion and flow ((Deen, 1998), Chapter 9). Assuming that a characteristic length L_{c} may be defined for the problem, Pe is defined as the ratio of the diffusion time scale T_{D}=L_{c}²/D to the advection time scale T_{A}=L_{c}/u where u is the velocity of the flow so Pe=T_{D}/T_{A}=uL_{c}/D. If Pe>>1 then advection dominates whereas if Pe<<1 then diffusion dominates.

In the present case there is a moving sink and a stationary medium; nevertheless, symmetry arguments suggest a calculation of Pe is applicable to the present problem, and indeed it has appeared before in discussions of morphogenesis (Howard et al., 2011). Assuming that a characteristic length for the present problem is L_{c}=100 μm then, using the above values for D and u, Pe=0.012, indicating that the motion is not likely to be important. As will be discussed later, the effective diffusion coefficient, Dₑ, may be less than D; if Dₑ=D/20, Pe will still be small and diffusion dominant.

Stationary Model with Localized Sink and Distributed Source

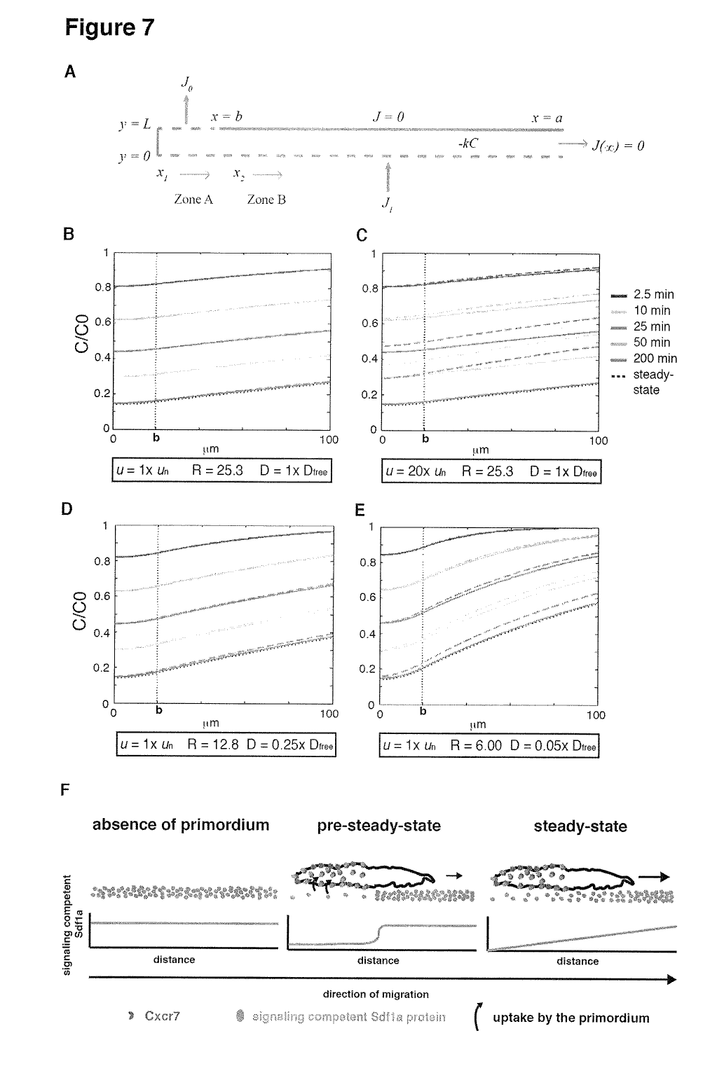

The geometry of the model is shown in FIG. 7. A narrow channel of diffusible chemokine with depth in y-axis of L (μm) sits above a layer of cells that produce the chemokine so that there is a constant flux J₁ (mol μm⁻² s⁻¹) into the channel at the lower boundary, y=0. The channel extends indefinitely to the right along the x-axis and is terminated on the left at x=0 by an impermeable wall. This is equivalent to the whole system as being reflected at x=0 with no wall. The top of the channel (y=L) is also reflecting (no flux) except for the interval [0<x<b, y=L] where the rear of the primordium forms a sink that absorbs chemokine with flux J₀ (mol μm⁻² s⁻¹) while the region [b<x<a, y=L] constitutes the front of the primordium where the chemokine gradient is sensed. Far from the sink (x≧a) there must be a constant concentration C₀ (mol μm⁻³) in the channel and this requires that the chemokine is degraded or otherwise cleared by a constant process that will be characterized here by a first-order rate constant k (s⁻¹). The z-axis is infinitely extended in both directions; because there is no flux in this axis, the width of the channel can be arbitrarily small and impermeable boundaries may be placed at any location.

The governing diffusion equation is

$$\frac{\partial C}{\partial t} = D \{ \frac{{\partial }^{2} C}{\partial {x}^{2}} + \frac{{\partial }^{2} C}{\partial {y}^{2}} \} - kC \qquad ( S 1 )$$

A full solution in this geometry is possible but tedious because the concentration will be distorted near the point (b, L). A detailed solution is not required, however, because the depth in the y-axis is small and averaging C in this axis suffices ((Deen, 1998), Chapter 9). The average is given by:

$$\overline{C} ( x ) = \frac{1}{L} {\int }_{0}^{L} C ( x , y ) d y .$$

Averaging Eq (S1) in this axis leads to

$$\frac{\partial \overline{C}}{\partial t} = D \{ \frac{{d}^{2} \overline{C}}{d {x}^{2}} + \frac{1}{L} \frac{\partial C}{\partial y} {\vert}_{0}^{L} \} - k \overline{C} . \qquad ( S 2 )$$

It is convenient to divide problem into two zones, with new space variables x₁, x₂, and use the standard definition of flux together with the boundary conditions described above, so that in

$$Zone A \text{:} 0 < {x}_{1} < b \text{:} {J}_{0} = - D \frac{\partial C}{\partial y} {\vert}^{L} \\ and$$

$${J}_{1} = - D \frac{\partial C}{\partial y} {\vert}_{0} \\ and in$$

$$Zone B \text{:} b < {x}_{2} < ∞ \text{:} 0 = - D \frac{\partial C}{\partial y} {\vert}^{L} \\ and again , \\ {J}_{1} = - D \frac{\partial C}{\partial y} {\vert}_{0} .$$

Inserting these boundary conditions in Eq (S2) and defining new variables C₁ and C₂ for the y-averaged concentrations in the two zones yields

$$\begin{matrix}\frac{\partial {C}_{1}}{\partial t} = D \frac{{\partial }^{2} {C}_{1}}{\partial {x}_{1}^{2}} - \frac{{J}_{0}}{L} + \frac{{J}_{1}}{L} - {kC}_{1} , & ( S 3 a ) \\ \frac{\partial {C}_{2}}{\partial t} = D \frac{{\partial }^{2} {C}_{2}}{\partial {x}_{2}^{2}} + \frac{{J}_{1}}{L} - {kC}_{2} . & ( S 3 b )\end{matrix}$$

The equations now involve only independent variables (x, t) so at the ends of the channel the following boundary conditions apply: Jₓ₁(0, t)=0, Jₓ₂(∞, t)=0. At the boundary between zones: Jₓ₁(b, t)=Jₓ₂(b, t) and C₁(b, t)=C₂(b, t).

This problem may be solved using the Laplace transform method where

$${C}^{*} ( x , s ) = {\int }_{0}^{∞} exp ( - st ) C ( x , t ) d t .$$

Then Eqs (S3a, b) become:

$$\begin{matrix}\frac{D}{k + s} \frac{{d}^{2} {C}_{1}^{*}}{d {x}_{1}^{2}} - \frac{{J}_{0} - {J}_{1}}{Ls ( k + s )} - {C}_{1}^{*} & ( S 4 a ) \\ \frac{D}{k + s} \frac{{d}^{2} {C}_{2}^{*}}{d {x}_{2}^{2}} + \frac{{J}_{1}}{Ls ( k + s )} - {C}_{2}^{*} & ( S 4 b )\end{matrix}$$

where starred variables (‘*’) denote the Laplace transforms of the concentrations and it is assumed that C₁(x₁, 0+)=C₂(x₂, 0+)=C₀ (see below for evaluation of C₀).

Eqs (S4a, b) are ordinary differential equations in either x₁ or x₂ and have standard solutions as the sum of exponentials with arguments ±\sqrt{(k+s)x/D}. Solving and applying boundary conditions defined above (after taking the Laplace transform where appropriate) and assuming that the source J₁ is on at all times while the sink J₀ commences at t=0, then performing the requisite algebra, yields solutions in Laplace space:

$$\begin{matrix}{C}_{1}^{*} ( {x}_{1} , s ) = \frac{1}{sL} [ \frac{{J}_{1}}{k} - \frac{{J}_{0}}{k + s} ( 1 - exp ( - \sqrt{\frac{k + s}{D}} b ) cosh ( \sqrt{\frac{k + s}{D}} {x}_{1} ) ) ] & ( S 5 a ) \\ {C}_{2}^{*} ( {x}_{2} , s ) = \frac{1}{sL} [ \frac{{J}_{1}}{k} - \frac{{J}_{0}}{k + s} sinh ( \sqrt{\frac{k + s}{D}} b ) exp ( - \sqrt{\frac{k + s}{D}} {x}_{2} ) ] . & ( S 5 b )\end{matrix}$$

By expanding the hyperbolic functions as exponentials and applying a succession of standard Laplace transform relations (Doetsch, 1971), the inverse Laplace transforms may be obtained:

$$\begin{matrix}{C}_{1} ( {x}_{1} , t ) = \frac{{J}_{1}}{kL} - \frac{{J}_{0}}{kL} ( 1 - exp ( - kt ) ) + \frac{{J}_{0}}{2 L} {\int }_{0}^{t} [ erfc ( \frac{b - {x}_{1}}{2 \sqrt{D ξ}} ) + erfc ( \frac{b + {x}_{1}}{2 \sqrt{D ξ}} ) ] exp ( - k ξ ) d ξ , & ( S 6 a ) \\ {C}_{2} ( {x}_{2} , t ) = \frac{{J}_{1}}{kL} - \frac{{J}_{0}}{2 L} {\int }_{0}^{t} [ erfc ( \frac{{x}_{2} - b}{2 \sqrt{D ξ}} ) - erfc ( \frac{{x}_{2} + b}{2 \sqrt{D ξ}} ) ] exp ( - k ξ ) d ξ . & ( S 6 b )\end{matrix}$$

It is clear that at t=0, C₁(x₁, 0)=C₂(x₂, 0)=C₀=J₁/kL, which is the steady concentration in the channel above the stripe representing a balance between the distributed source J₁ and the degradation process characterized by the rate constant k. Therefore, Eqs (S6a, b) may be normalized by dividing through by C₀ to obtain

$$\begin{matrix}\frac{{C}_{1}}{{C}_{0}} = 1 - R ( 1 - exp ( - kt ) ) + \frac{Rk}{2} {\int }_{0}^{t} [ erfc ( \frac{b - {x}_{1}}{2 \sqrt{D ξ}} ) + erfc ( \frac{b + {x}_{1}}{2 \sqrt{D ξ}} ) ] exp ( - k ξ ) d ξ & ( S 7 a ) \\ \frac{{C}_{2}}{{C}_{0}} = 1 - \frac{Rk}{2} {\int }_{0}^{t} [ erfc ( \frac{{x}_{2} - b}{2 \sqrt{D ξ}} ) - erfc ( \frac{{x}_{2} + b}{2 \sqrt{D ξ}} ) ] exp ( - k ξ ) d ξ & ( A 7 b )\end{matrix}$$

where R=J₀/J₁ is the ratio of the localized sink flux to the distributed source flux.

It is also useful to state the steady-state solutions to the problem i.e. the result as t→∞. This may be derived immediately from Eqs (S5a, b) by noting that

$$C ( x , ∞ ) = {lim}_{s \to 0} {sC}^{*} ( x , s )$$

(Doetsch, 1971) to obtain

$$\begin{matrix}\frac{{C}_{1} ( {x}_{1} , ∞ )}{{C}_{0}} = 1 - R ( 1 - exp ( - \sqrt{\frac{k}{D}} b ) cosh ( \sqrt{\frac{k}{D}} {x}_{1} ) ) & ( S 8 a ) \\ \frac{{C}_{2} ( {x}_{2} , ∞ )}{{C}_{0}} = 1 - R sinh ( \sqrt{\frac{k}{D}} b ) exp ( - \sqrt{\frac{k}{D}} {x}_{2} ) . & ( S 8 b )\end{matrix}$$

The experimental data indicates that the baseline concentration of chemokine under the sink in the steady-state, C_{b}, is about 0.14×C₀. In order to start the gradient from that value in each calculation, Eq (S8a) was used to define R as

R(C_{b}/C₀)=(1−C_{b}/C₀)/(1−exp(−b\sqrt{k/D})).

### Moving Coordinates

As noted above, the Peclet number indicates that the motion of the primordium contributes little to the shape of the gradient, which is dominated by diffusion for the values of D and u in this situation. The effect of movement may be incorporated in Eqs (S7a, b) by appropriate analysis, which will not be described in detail here. Suffice to note that in the usual advection or flow problem, the medium moves and a source is either stationary or carried with the flow and a transformation is made to moving coordinates where the diffusion problem is solved. Here the medium is stationary, while the sink moves, and it is desired to see the gradient from the perspective of the moving primordium, where the sink is located. It may be shown that this is accomplished by the following substitutions: x→η+uτ; t→τ in Eqs (S7a, b) where η and τ are the coordinates in the moving frame. This substitution has been made to calculate some of the results shown in FIG. 6H and FIG. 7.

### Calculations

All equations were evaluated using Matlab 2012b (MathWorks, Natick, Mass.). The integrals in Eqs (S7a, b) were performed with the built-in function ‘integral’, which uses global adaptive quadrature, with default tolerances.

### Results and Discussion

In order to make the calculations shown in FIG. 6H and FIG. 7 it was necessary to choose a value for the rate constant that defines the degradation or clearance process. Our calculations and the literature (Kicheva et al., 2007) suggest that k=0.0003 s⁻¹ is a reasonable value. Based on extensive calculations (not shown) the present inventors found that the value of k largely determined how rapidly the chemokine gradient reached a steady-state and that a value of k=0.0003 s⁻¹ was appropriate for the ˜200 min time frame observed experimentally.

Panel A of FIG. 7 shows Eqs (S7a, b) evaluated at various times for D=100 μm² s⁻¹, the assumed free diffusion coefficient for Sdf1. The solid lines show the solution for u=0 (Eqs S7a, b); the effect of motion with u=0.7 μm min⁻¹ is shown as dashed lines, which are effectively superimposed on the u=0 lines here, showing that the effect of movement is imperceptible. For the part of curve beyond the sink (which extends from 0 to 20 μm here; a longer sink does not have much effect on the results), the gradient is a straight line indicating that the argument of the exponential in Eq (S7b) is sufficiently small that it may be represented by the linear part of the series expansion. It is seen that by 200 min after activating the sink, the gradient has reached the steady-state curve, shown as a dotted black line (Eqs S8a, b). To show that a sufficiently high primordium velocity can affect the gradients, Panel B shows the effect increasing u by 20×. Now a steady-state is apparently reached by about 120 min and the gradients are steeper reflecting the fact that the primordium is ‘pushing into’ the reservoir of chemokine in the stripe. Note, however, that even this non-physiological velocity does not affect the overall shape of the gradient.

Returning to Panel A it is apparent that the gradient evolves to a steady-state in the requisite time and is linear, but the slope is smaller than that observed experimentally. Panels C and D explore the possibility that the reason for the shallow slope is that the assumed value of the diffusion coefficient is inappropriate. In many systems the chemokine or morphogen interacts with the extracellular matrix, most often with the heparin sulfate component, which may result in an effective diffusion coefficient that is considerably smaller than D. In Panel C the effective diffusion coefficient, Dₑ, is set at D/4 while in Panel D it is D/20. It is evident that as Dₑ decreases, the slope of the gradient increases and in Panel D it is close to the slope observed experimentally. This suggests that a reduced Dₑ is indeed at work here. Looking again at the last panel it is seen that the gradient is now departing from a straight line as the exponential nature of the curve becomes more apparent (actually the whole curve, if extended beyond 100 μm would be sigmoidal as it asymptotically approached unity). It is also apparent that, with a reduced diffusion coefficient, the effect of motion, though still small, begins to become more evident.

Comparison to Crick model

The model proposed by Crick (1970) for diffusion embryogenesis is widely cited. It envisages a discrete source of morphogen and discrete sink separated by 50-100 cells through which the morphogen travels with an effective diffusion coefficient. The source is represented by a constant concentration boundary condition and the sink is represented by a zero concentration boundary condition. This type of problem was solved earlier by Carslaw and Jaeger ((Carslaw and Jaeger, 1959), section 3.4) from which the time evolution may be easily derived without ad hoc arguments. By necessity Crick's model arrives at a linear gradient in the steady-state, although the time-evolution of the gradients is quite complex in shape and would not mimic the present experimental results because of Crick's fixed concentration at the sink. In the present situation, the sink might be approximated by a point-sink but the need to have a stable stripe of chemokine over a very long distance excludes a localized source while also necessitating a degradation process. The present model only generates a linear steady-state gradient when the gradient is quite shallow but allowing the primordium to release an additional substance that inhibits chemokine production might generate a steeper, linear gradient.

### Results

Sdf1a-GFP Levels are Distributed evenly along the Migratory Route of the Primordium

To analyze the distribution of Sdf1a protein along the migratory route of the primordium the present inventors generated a transgenic line (tg(sdf1a:sdf1a-GFP)) that expresses Sdf1a fused to green fluorescent protein (GFP) from a bacterial artificial chromosome (BAC). This BAC includes the sdf1a exons and introns, a 55 kb sequence upstream of the start codon and a 30 kb sequence downstream of the stop codon (FIG. S1A). The transgene recapitulates the endogenous sdf1a mRNA expression pattern and restores primordium migration in sdf1a mutant embryos demonstrating that it is functional. The tg(sdf1a:sdf1a-GFP) line was used to examine the distribution of Sdf1a-GFP protein in wild type embryos. Sdf1a-GFP protein is distributed evenly along the migration route of the primordium and is confined to the immediate vicinity of the cells that produce it. The intensity of Sdf1a-GFP on the stripe underneath the primordium was quantitated and no detectable difference in the levels of the chemokine between the front and rear of the primordium was observed (FIG. 2A). However, close inspection reveals that cells in the rear of the primordium sequester small amounts of Sdf1a-GFP, which appear as discrete puncta (FIG. 2C). Quantification of the number and intensity of Sdf1a-GFP puncta from primordia of multiple embryos confirms that cells in the rear of the primordium internalize more Sdf1a-GFP than the cells in the front of the primordium (FIG. 2E). This raises the possibility that the rear of the primordium reduces the concentration of Sdf1a beneath it through protein sequestration, indicating the primordium is capable of locally modifying the levels of chemokine on its path. However, the Sdf1a-GFP uptake by the rear of the primordium represents only 1% of the total Sdf1a-GFP signal (FIG. 2F) and is thus within the noise margin (SEM 18%) of Sdf1a-GFP intensity measurements made from the stripe beneath the primordium. This suggests that the migrating primordium is modifying the chemokine levels on the stripe around its rear.

A Novel in vivo Sdf1-Signaling Sensor

It is possible that the primordium detects and responds to a gradient of Sdf1a that remains undetected when measuring the total amount of Sdf1a-GFP protein along the stripe. To investigate this possibility, the present inventors developed an in vivo Sdf1-signaling sensor designed to measure the levels of signaling competent Sdf1a that the primordium perceives. Since the binding of SDF1 to CXCR4 triggers internalization of the receptor from the cell membrane followed by degradation (Marchese and Benovic, 2001; Marchese et al., 2003; Minina et al., 2007), the present inventors reasoned that the levels of Cxcr4b on the cell membrane should correlate inversely to extracellular, signaling competent Sdf1 protein levels. To test this idea, the monomeric red fluorescent protein Kate2 was fused to the C-terminus of Cxcr4b (Cxcr4b-Kate2) and this fusion protein was expressed from the cxcr4b promoter. As an internal reference, a membrane tethered GFP (memGFP) that is co-translated from the same transcript through an internal ribosomal entry site (IRES) was co-expressed (FIG. 3A and S2A). This signaling sensor recapitulates the expression of cxcr4b and rescues primordium migration in cxcr4b mutant embryos, demonstrating that it is functional. Since ligand binding causes receptor internalization, the ratio of the red fluorescence from the Cxcr4b-Kate2 fusion protein to the green fluorescence from the memGFP on the membrane of a cell (FmemRed/FmemGreen) should represent a quantitative readout of the amount of signaling competent Sdf1a protein to which the cell is exposed (FIG. 3B). The present inventors tested the relationship between Sdf1a protein levels and the Sdf1-signaling sensor in three ways. First, in the absence of Sdf1a protein, the membranes of cells within the primordium exhibit high levels of Cxcr4b-Kate2, resulting in an average FmemRed/FmemGreen ratio of 2.6 (FIGS. 3D and 3E, column 4). Second, upon global Sdf1a over-expression from an inducible heat-shock promoter (tg(hsp70:sdf1a)), Cxcr4b-Kate2 is found primarily inside the cell rather than on the cell membrane, resulting in an average FmemRed/FmemGreen ratio of 0.2 (FIGS. 3D and 3E, column 5). Third, injection of increasing amounts of a translation-blocking sdf1a morpholino progressively shifts the FmemRed/FmemGreen ratios across the primordium to higher values, consistent with progressively decreasing levels of Sdf1a, and the overall shift of the FmemRed/FmemGreen ratios is directly proportional to the amount of sdf1a morpholino injected (FIG. 3C). Thus, the ratio of FmemRed/FmemGreen reported by the Sdf1-signaling sensor is linearly related to the levels of signaling competent Sdf1 protein that the primordium perceives during migration.

A Linear Sdf1-Signaling Gradient across the Primordium

Using this sensor, the present inventors detected an Sdf1-signaling gradient across the anterior-posterior axis of the migrating primordium in live, 36 hpf wild-type embryos (FIGS. 3D and 3E, column 2). The gradient begins at the leading edge of the primordium at a mean FmemRed/FmemGreen of below 0.6, increases fairly linearly by 1.2%/μm for the first 100 μm and plateaus at a mean FmemRed/FmemGreen of 2.3 in the rear of the primordium (FIG. 3E, column 2). The linear gradient moves with the primordium throughout its migration, remaining remarkably constant in shape and amplitude over time (FIGS. 3D and 3E, columns 1-3). Moreover, the gradient is absent in Sdf1a mutant embryos (FIG. 3E, column 4) and is rapidly abolished upon global over-expression of Sdf1a from a heat-shock inducible promoter (FIG. 3E, column 5), confirming that the signaling gradient depends on Sdf1a protein levels. To approximate the lower and upper limits of Sdf1-signaling in the primordium, the present inventors compared the mean FmemRed/FmemGreen in sdf1a mutant embryos and embryos that globally over-express Sdf1a. The maximal difference in chemokine signaling observed between these two scenarios is 2.4 ratio units (mean FmemRed/FmemGreen of 2.6 and 0.2, respectively, in FIG. 3E, columns 4 and 5). When compared to the maximal signaling difference between the front and back of wild-type primordia of 1.7 ratio units (mean FmemRed/FmemGreen of 0.6 and 2.3, respectively), this indicates that 36 hpf wild-type embryos use 71% of the Sdf1-signaling range to ensure proper primordium migration.

The Sdf1-signaling gradient observed across the primordium—high signaling in the leading cells and low signaling in the trailing cells—suggested that a graded distribution of Sdf1a continuously confers directional information to the migrating primordium. Results from a previous study demonstrated that ectopic sources of the chemokine Sdf1b, the protein encoded by a closely related paralog of sdf1a, can attract the primordium (Li et al., 2004). Since Sdf1b is not expressed along the migratory route of the primordium and dispensable for its migration, the present inventors hypothesized that Sdf1a, like Sdf1b, can lure the primordium off course when expressed ectopically, therefore acting as an instructive rather than permissive guidance cue. The present inventors tested this hypothesis in two ways. First, Sdf1a was over-expressed from a heat-shock promoter during primordium migration in embryos carrying the cldnB:lynGFP transgene or the Sdf1-signaling sensor. In response to global over-expression of Sdf1a, the primordium exhibits uniformly high levels of Cxcr4b-Kate2 internalization, rounds up and ceases to migrate. This stands in marked contrast to primordia in heat-shocked control embryos that report a steady, linear signaling gradient, maintain an elongated morphology, and continue to migrate. Second, small, Sdf1a-misexpressing clones were generated along the migratory route of the primordium or within the primordium in tg(cldnB:lynGFP) embryos by blastomere transplantation. Clones positioned dorsal or ventral to the normal migratory route were able to attract the primordium, sending it off course, while clones within the primordium caused it to round up and stall. In summary, these findings are consistent with a mechanism in which a signaling competent population of Sdf1a acts as an attractive cue that is produced uniformly along the migration route but is continuously converted into a graded distribution that confers directional information to a migrating cohort of cells.

Cxcr7 Sequesters Sdf1a Protein in the Rear of the Primordium

Results presented herein that the rear of the primordium sequesters Sdf1a-GFP protein (FIG. 2C) and perceives lower levels of Sdf1 than the front (FIGS. 3D and 3E, columns 1-3) suggest that it continuously clears signaling competent Sdf1a protein from the region underneath it. Previous studies have proposed that the alternate SDF1 receptor CXCR7 can act as a scavenger receptor for chemokines (Boldajipour et al., 2008; Sánchez-Alcañiz et al., 2011). Consistent with this proposition, cxcr7b is expressed in the rear of the primordium (FIG. 1B, column 3), and is required for its migration (FIGS. 1A and 1D) (Dambly-Chaudière et al., 2007; Valentin et al., 2007). Primordia in cxcr7b mutant embryos exhibit slowed migration or stalling (Valentin et al., 2007). Interestingly, the present inventors find that cxcr7a the second ortholog of CXCR7 in zebrafish is also expressed in the rear of the primordium (FIG. 1B, column 2). In embryos injected with morpholinos that block translation of cxcr7a transcripts, the primordium migrates less efficiently compared to wild type (FIGS. 1A and 1D). Compromising the function of both CXCR7 orthologs enhances these migration defects (FIGS. 1A and 1D), often resulting in complete stalling of the primordium, a defect that is comparable to what the present inventors observe in primordia of sdf1a mutant embryos (FIG. 1A). However, in contrast to the rounded morphology that the primordium assumes in sdf1a mutant embryos (FIG. 1A), primordia deficient in cxcr7a and cxcr7b (collectively referred to as Cxcr7) often extend in multiple directions. Thus, Sdf1a protein sequestration by Cxcr7 is a plausible mechanism for the generation and maintenance of a chemokine attractant gradient across the migrating primordium. To test this, the present inventors measured Sdf1a-GFP protein uptake by the primordium in cxcr7 deficient and cxcr7b over-expressing embryos. Consistent with the hypothesis, the present inventors find that cxcr7 deficient primordia fail to sequester Sdf1a-GFP protein in the rear of the primordium, in contrast to wild-type primordia that show significant uptake in this region (FIG. 2C-2F). The number and intensity of the Sdf1a-GFP puncta are markedly reduced in cxcr7 deficient primordia (FIGS. 2D and 2E) suggesting that Cxcr7 is required for chemokine sequestration. Conversely, over-expression of Cxcr7b from a heat-shock inducible promoter causes the primordium to assume a rounded morphology similar to that observed in sdf1a mutant embryos and to decelerate. Sdf1a-GFP protein levels on the stripe outside the primordium are reduced by 29% in these embryos (FIG. 2B), indicating that Cxcr7 activity promotes removal of Sdf1a from the stripe.

Cxcr7 Generates an Sdf1-Signaling Gradient across the Primordium

The present inventors next tested whether Cxcr7-mediated Sdf1a protein sequestration in the rear of the primordium is responsible for generating the Sdf1-signaling gradient across the primordium. Consistent with the intermediate and variable migration defects observed in embryos deficient for either cxcr7a or cxcr7b, the present inventors find that Sdf1-signaling is increased specifically in the rear of the primordium in the absence of cxcr7a or cxcr7b activity compared to wild-type controls (FIGS. 4A and 4B, columns 1-3), resulting in a 31% or 40% reduction in the steepness of the Sdf1-signaling gradient across the primordium, respectively. These findings indicate that both CXCR7 orthologs contribute to the local clearance of signaling competent Sdf1a protein and, thus, to the generation of the signaling gradient. Indeed, impairing both cxcr7a and cxcr7b activity in the same embryo increases Sdf1-signaling in the rear to levels that are normally only observed in the front of the primordium (FIGS. 4A and 4B, column 4). Importantly, this increase of Sdf1-signaling in the rear of the primordium of cxcr7 deficient embryos requires Sdf1a activity since Sdf1-signaling in primordia of embryos lacking cxcr7 and sdf1a resembles Sdf1-signaling in primordia of embryos mutant for sdf1a alone (FIGS. 4A and 4B, column 5). Conversely, in embryos that over-express Cxcr7b from a heat-shock inducible promoter, Sdf1-signaling is reduced throughout the primordium (FIGS. 4A and 4B, column 6).The absence of the Sdf1-signaling gradient in cxcr7 deficient primordia resembles the scenario in which Sdf1a is over-expressed globally (FIGS. 3D and 3E, column 5), while the high signaling levels observed across the primordium upon global Cxcr7b over-expression are similar to what the present inventors observed in sdf1a mutant primordia (FIGS. 3D and 3E, column 4), suggesting that Cxcr7 activity correlates inversely with signaling competent Sdf1a levels. Furthermore, the abrogation of the Sdf1-signaling gradient in cxcr7 deficient embryos enables the relative quantification of signaling competent Sdf1a levels outside the primordium. In the absence of cxcr7 activity, the mean FmemRed/FmemGreen in the primordium should correspond to the unaltered levels of signaling competent Sdf1a on the stripe (C₀), and the mean FmemRed/FmemGreen in sdf1a mutant embryos should correspond to the absence of signaling competent Sdf1a. Thus, the combined activities of Cxcr7a and Cxcr7b reduce the signaling competent Sdf1a beneath the rear of the primordium to 0.14×C₀, while the front of the primordium perceives C₀.

In summary, these observations demonstrate that Cxcr7a and Cxcr7b continuously sequester Sdf1a protein in the rear of the primordium. This sequestration results in an 86% reduction in the local concentration of signaling competent Sdf1a in the rear of the primordium, which in turn generates the difference in chemical potential required for the formation of a linear gradient of the attractant along the migration route, which is essential for proper primordium migration.

Cxcr7 Shapes the Sdf1-Signaling Gradient on the Tissue Level

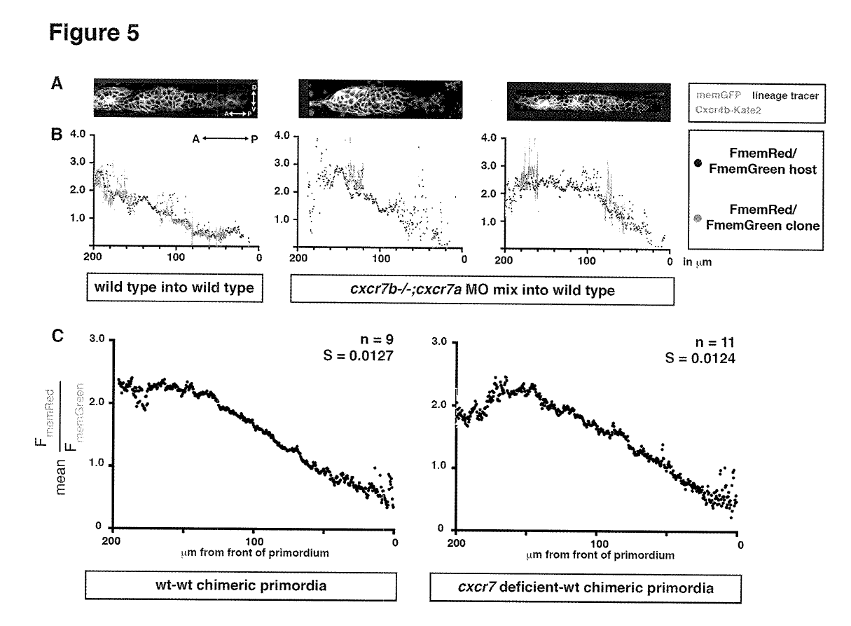

Cxcr7 could sculpt the chemokine gradient across the primordium through local competition with Cxcr4b for Sdf1a protein on the cell membranes of individual cells or through global chemokine clearance in the rear of the primordium. To distinguish between these possibilities, the present inventors used cell transplantation to generate chimeric primordia composed of wild-type and cxcr7 deficient cells and compared the FmemRed/FmemGreen ratios within and outside the clones. Placement of cxcr7 deficient cells in the rear of a wild-type primordium does not result in increased internalization of Cxcr4b-Kate2 selectively in the cxcr7 deficient clones when compared to adjacent wild-type cells or control chimeras (FIGS. 5A and 5B). This indicates that, although Cxcr7a and Cxcr7b clear Sdf1a protein locally, the reduction of Sdf1-signaling in the rear of the primordium is generated at the level of the tissue rather than the individual cell.

A Steady-State Sdf1-Signaling Gradient Guides the Migrating Primordium

Given sufficient time, the shape and amplitude of a signaling molecule gradient will reach steady-state in many scenarios (Muller et al., 2013; Wartlick et al., 2009). However, signaling processes often occur within a few hours, and it is unclear if gradients can reach steady-state within such short time frames, and in turn, if cells interpret pre-steady-state or steady-state signaling gradients in vivo. To address these questions, the present inventors analyzed the formation of the Sdf1-signaling gradient over time. A brief, heat-shock induced pulse of global Sdf1a protein expression causes internalization of Cxcr4b-Kate2 throughout the primordium, flattens the Sdf1-signaling gradient and decelerates the primordium. The gradient begins to recover ˜5-6 hours after the heat-shock and converges to the linear shape that is observed across wild-type primordia (FIG. 6A-6C). Concurrent with recovery of the gradient, the rounded primordium elongates and resumes normal migration. Analysis of this recovery reveals a sequence of three distinct states of Sdf1-signaling across the primordium that result in reestablishment of the signaling gradient: At ˜5 hours post heat-shock, the Sdf1-signaling gradient across the primordium is absent (FIG. 6A). At ˜7-8 hours post heat-shock, Sdf1-signaling is reduced specifically in the rear of the primordium, resulting in a non-linear, sigmoidal Sdf1-signaling gradient (FIG. 6B). Over the next ˜2 hours, this sigmoidal gradient equilibrates across the primordium to yield a steeper, linear gradient that resembles the gradients observed in wild-type primordia and remains relatively stable until the end of the imaging period (FIGS. 6C and 6G), indicating that it has reached a steady-state. Importantly, the time required for re-establishment of the Sdf1-signaling gradient across the primordium depends on cxcr7 activity. In cxcr7b mutant embryos, a genetic scenario in which the slope of the Sdf1-signaling gradient across the primordium during migration under normal conditions is reduced by 40% (FIGS. 4A and B, column 3), the gradient remains flat ˜10 hours following a similar pulse of global Sdf1a protein expression (FIG. 6A-6C). These observations are consistent with a mechanism in which Cxcr7-mediated sequestration potentiates the formation of a steady-state Sdf1-signaling gradient across the primordium that mediates proper migration.

Mathematical Model for Gradient Formation by a Moving Sink

The local sequestration of Sdf1a protein in the rear of the migrating primordium resembles a dynamic version of the source-sink model described by Crick (Crick, 1970), in which the stripe of sdf1a mRNA expressing cells generates a reservoir of Sdf1a along the migration route while a localized sink in the rear of the primordium locally degrades Sdf1a. As the primordium migrates, it removes Sdf1a from the stripe beneath its rear causing Sdf1a to diffuse in from the chemokine reservoir in front of it, where the Sdf1a reservoir acts as a localized source by presenting the attractant at a constant concentration. Crick showed that a freely diffusing molecule produced by a localized source and degraded by a localized sink should result in a linear gradient at steady-state. Consistent with this prediction, the Sdf1-signaling gradient across wild-type primordia appears linear (FIG. 3E). To test whether this is a plausible scenario, the present inventors modeled these dynamics under two assumptions. First, the flux of Sdf1a from a distributed source of chemokine producing cells is balanced by its degradation to yield a constant reservoir of Sdf1a. Second, the rear of the primordium clears Sdf1a at a constant flow rate (FIG. 7A). Consistent with our analysis of the Sdf1-signaling gradient kinetics and the estimated Peclet number of 0.012—a measure for whether a system is dominated by diffusion or flow—this model predicts that the primordium migration velocity of ˜0.7 μm/min does not contribute significantly to the formation of the gradient (FIGS. 7B and 7C). Moreover, this model shows that a stable, linear, gradient can form in 0.5 to 3 hours and is little perturbed by the motion of the primordium (FIG. 7B-7E). However, the model predicts a shallower signaling gradient across the primordium (FIG. 7B) than that observed in vivo (FIG. 3E, column 1-3), perhaps reflecting hindered Sdf1a diffusion mediated by molecules in the extracellular matrix that can bind the chemokine, a scenario that is known to reduce the effective diffusion coefficient (Crank (1975), Chapter 14). Three of the present observations are consistent with this idea. First, Sdf1a protein is produced by the stripe of chemokine expressing cells throughout the 20-hour migration period but does not diffuse to detectable levels into adjacent tissues, suggesting that Sdf1a protein is retained close to its source. Second, only 1% of the total Sdf1a-GFP protein on the stripe is sequestered through Cxcr7 in the rear of the primordium (FIG. 2F). This suggests that a large fraction of the chemokine is bound to the extracellular matrix, a proposition that has also been put forward for other secreted signaling molecules (Muller et al., 2013). Importantly, prolonged global over-expression of Cxcr7b results in removal of 29% of the total Sdf1a-GFP protein from the stripe (FIG. 2B), indicating that a larger fraction of Sdf1a protein than the 1% sequestered by the primordium is present but not accessible to Cxcr7 in the rear of the primordium. Third, the kinetics of the Sdf1-gradient formation are approximated by the model when reducing the free diffusion coefficient of Sdf1 by a factor of 20 (FIGS. 7D and 7E). In summary, this modeling analysis supports the plausibility of a scenario in which a localized Cxcr7-mediated sink activity combined with hindered Sdf1a protein diffusion generates a linear and stable Sdf1-signaling gradient across the primordium.

Steepness of Sdf1-Signaling Gradient Correlates with Efficient Primordium Migration

Theoretical considerations and in vitro experiments have suggested that increasing the steepness of an attractant gradient can promote directionality and motility (Fisher et al., 1989; Hatzikirou and Deutsch, 2008; Keller and Segel, 1971). To test this model in vivo, the present inventors followed the recovery of the Sdf1-signaling gradient and primordium migration speed following exposure to a global pulse of Sdf1a expression. By comparing the average slope of the signaling gradient and the average speed of the primordium (FIG. 6D-6F), the present inventors found that when the slope of the Sdf1-signaling gradient is at or above ˜46% of its steady-state value (470 min in FIG. 6F), the speed of the primordium stabilizes at ˜0.7 μm/min (FIG. 6F), which is similar to the speed observed in wild-type primordia (Haas and Gilmour, 2006). During this recovery period, the Sdf1-signaling gradient increases fairly linearly (FIG. 6D) until it stabilizes at steady-state. When the gradient is less than ˜46% of the steady-state value, however, the speed and directionality of the primordium are unpredictable (FIG. 6F), with primordia either stalling or exhibiting serpentine movement rather than straight migration. These observations are consistent with the idea that the steepness of an attractant gradient instructs both speed and directionality of migration in vivo.

Exemplary Stable Cell Line for Screening Assays

In order to test whether the human version of the Sdf1-signaling sensor responds to human SDF1 in a similar fashion to that of the fish version of the Sdf1-signaling sensor, the present inventors generated a Flp-In™ T-REx™ 293 cell line that expresses human CXCR4 fused to the RFP variant Kate2 followed by an IRES and a membrane-tethered GFP from a tetracycline-inducible promoter. Because the flippin technology allows one to generate a single copy transgene in a specific genomic location of HEK 293 cells, expression levels from the human Sdf1-signaling sensor are fairly uniform (FIGS. S4A and C). Treating the Flp-In™ T-REx™ 293 cell line carrying the human Sdf1-signaling sensor with recombinant human SDF1 induces internalization of CXCR4-Kate2 within 40 minutes (FIG. S4D) but not in vehicle treated controls (FIG. S4B). This observation indicates that the human Sdf1-signaling sensor responds to human SDF1 and this system, therefore, offers a screening assay for identifying CXCR4 agonists, antagonists and molecular factors that regulate CXCR4 internalization and activation.

### Discussion

The development of a linear Sdf1-signaling sensor in combination with BAC transgenesis, quantitative imaging and mathematical modeling allowed the present inventors to analyze the kinetics and dynamics of an Sdf1-signaling gradient in a living animal. The Sdf1-signaling gradient requires about 200 minutes to form, provides guidance to the migrating primordium at steady-state and is linear in shape with a slope of 7% change in signaling across cells. The comparison of the signaling competent Sdf1a protein to the total Sdf1a protein reveals that only a small fraction of the total Sdf1a protein pool is competent to signal. It is likely that these features—time of gradient formation, stable steady-state gradient, small pool of signaling competent molecules—are general features of many signaling gradients in animals. For example, immune cells rely on cytokines for recruitment to sites of infection and detect similar shallow gradients of 3% change in attractant concentration across cells (Parent, 1999; Tranquillo et al., 1988).

Moreover, the present study shows that collectively migrating cells generate their own attractant gradient across themselves that they then use to direct their migration along a uniform source of attractant (FIG. 7F). It is conceivable that ligand sequestration by a subset of cells in a migrating cluster represents a more general mechanism of generating or maintaining a gradient of an attractant—or any signaling molecule—in order to provide directional and/or positional information to cells and tissues. Many other tissues such as sprouting blood vessels, epithelia and metastasizing tumors exhibit collective migration (Friedl and Gilmour, 2009; Montell, 2008; Rørth, 2009). Thus, modulating the availability of a guidance cue by the migrating collective itself represents an elegant mechanism of cell guidance.

Guidance of Migrating Cells by Shallow Attractant Gradients

In vitro studies using Dictyostelium and neutrophils have shown that singly migrating cells require a 3% difference in concentration between the front and the back of the cell for efficient directional migration (Fisher et al., 1989; Mato et al., 1975). This is surprisingly similar to the 7% difference in Sdf1-signaling observed across the front to the back of a cell in the lateral line primordium, suggesting that both in vitro and in vivo shallow gradients are sufficient for efficient directional migration. Since most scenarios involving a local attractant source yield non-linear gradients whose slope is shallow at a point away from the source and steeper closer to the source (Wartlick et al., 2009), an increased sensitivity of cells to detect small differences in concentration of the attractant is essential to migrate towards the attractant source from a point away from it.

One advantage of collectively migrating cells is that they can potentially compare differences in attractant concentration sensed by cells at the front and at the rear of the collective to polarize the tissue towards higher attractant concentrations (Rørth, 2007). The induction of polarity across collectively migrating border cells in flies by local activation of Rac supports this idea (Wang et al., 2010). However, reducing the difference in Sdf1-signaling across a cell within the primordium to 3% results in inefficient migration and stalling of the primordium, although there still exists a 40% difference in Sdf1-signaling from the front to back across the primordium in cxcr7b mutant embryos. Additionally, placing a few wild-type cells in the front of a cxcr4b mutant primordium restores primordium migration, but less efficiently than in wild type (Haas and Gilmour, 2006). These observations suggest that unlike border cells in flies, the primordium does not compare concentrations of the attractant across the collective to enhance its ability to detect attractant gradients.

Kinetics and Dynamics of Signaling Gradients

Signaling molecules disperse away from their source through a complex environment to pattern a field of cells or to provide guidance to migrating cells. The signaling range depends on the time the signaling molecules have to disperse and the ability of the signaling molecules to move through the tissue (Müller and Schier, 2011). For many scenarios with constant production, diffusion and clearance rates, the distribution of signaling molecules will converge towards a stable gradient (constant amplitude and shape) over time (Muller et al., 2013; Wartlick et al., 2009). Measurements of the total pool of fluorescently tagged and over expressed signaling molecules indicates that it takes 30 minutes (in the case of nodal (Muller et al., 2012)) to 3 to 4 hours (in the cases of FGF (Yu et al., 2009) and dpp (Entchev et al., 2000; Teleman and Cohen, 2000)) for the signaling gradient to reach steady state. This is surprisingly similar to the time it takes for the signaling gradient of untagged, endogenous Sdf1 to converge towards steady state, given that the distribution of the pool of total signaling molecules and the pool of signaling competent signaling molecules do not necessarily need to display similar gradient kinetics.

The movement of signaling molecules through tissues is slowed down by obstacles that increase the path length of the moving molecule and by transient binding to the extracellular matrix. This reduces the global diffusivity of the signaling molecule and the time it takes for the gradient to converge towards steady state (Muller et al., 2013). The FGF gradient in the early zebrafish embryo, for example, approaches steady state over a period of 3 to 4 hours instead of less than an hour as predicted for freely diffusing FGF, suggesting that the movement of FGF is hindered by transient binding to extracellular molecules (Muller et al., 2013; Yu et al., 2009). Similarly, the shape of the gradient of signaling competent Sdf1 suggests that the chemokine is hindered in its diffusivity, a supposition supported by the observation that only a small fraction of the total Sdf1a protein pool is competent to signal. Although this might depend on the signaling molecule and the tissue context, these observations are consistent with the idea that a large fraction of the signaling molecules are bound to extracellular molecules at any given time and only a small fraction is competent to engage in signaling.

Self-Generated Attractant Gradients

The primordium generates an Sdf1-signaling gradient across itself that it then uses to direct its migration along a uniform source of attractant (FIG. 7F). This solution to the problem of how to guide cells over long distances is different from what is employed during tracheal cell migration in flies, for example, where a group of cells follows a constantly shifting local FGF source (Ghabrial et al., 2003). One advantage of supplying an attractant from a constant stripe-source rather than a local but constantly shifting source could be the reduced time it takes to establish a stable gradient. Shifting sources require time for initiation of gene expression, secretion of the attractant and establishment of a new gradient, all of which is not necessary for attractants emanating from a stripe-source. Thus, in contrast to gradients that guide slowly migrating cells such as tracheal cells (˜16 μm/hour) (Caussinus et al., 2008), it is likely that stable gradients for rapidly migrating cell collectives such as the primordium (˜50 μm/hour) can only be generated from a long, constant stripe-source rather than a shifting local source.

### REFERENCES

Affolter, M., and Caussinus, E. (2008). Tracheal branching morphogenesis in Drosophila: new insights into cell behaviour and organ architecture. Development 135, 2055-2064.
Aman, A., and Piotrowski, T. (2010). Cell migration during morphogenesis. Dev Biol 341, 20-33.
Boldajipour, B., Mahabaleshwar, H., Kardash, E., Reichman-Fried, M., Blaser, H., Minina, S., Wilson, D., Xu, Q., and Raz, E. (2008). Control of Chemokine-Guided Cell Migration by Ligand Sequestration. Cell 132, 463-473.
Cai, H., and Devreotes, P. N. (2011). Moving in the right direction: How eukaryotic cells migrate along chemical gradients. Semin Cell Dev Biol 1-8.
Caussinus, E., Colombelli, J., and Affolter, M. (2008). Tip-Cell Migration Controls Stalk-Cell Intercalation during Drosophila Tracheal Tube Elongation. Curr Biol 18, 1727-1734.
Crick, F. (1970). Diffusion in embryogenesis. Nature 225, 420-422.
Dambly-Chaudière, C., Cubedo, N., and Ghysen, A. (2007). Control of cell migration in the development of the posterior lateral line: antagonistic interactions between the chemokine receptors CXCR4 and CXCR7/RDC1. BMC Dev Biol 7, 23.
David, N. B., Sapède, D., Saint-Etienne, L., Thisse, C., Thisse, B., Dambly-Chaudière, C., Rosa, F. M., and Ghysen, A. (2002). Molecular basis of cell migration in the fish lateral line: role of the chemokine receptor CXCR4 and of its ligand, SDF1. Proc Natl Acad Sci USA 99, 16297-16302.
Entchev, E. V., Schwabedissen, A., and Gonzalez-Gaitan, M. (2000). Gradient formation of the TGF-beta homolog Dpp. Cell 103, 981-991.
Fisher, P. R., Merkl, R., and Gerisch, G. (1989). Quantitative analysis of cell motility and chemotaxis in Dictyostelium discoideum by using an image processing system and a novel chemotaxis chamber providing stationary chemical gradients. The Journal of Cell Biology 108, 973-984.
Friedl, P., and Gilmour, D. (2009). Collective cell migration in morphogenesis, regeneration and cancer. Nat Rev Mol Cell Biol 10, 445-457.
Ghabrial, A., Luschnig, S., Metzstein, M. M., and Krasnow, M. A. (2003). Branching Morphogenesis of the Drosophila Tracheal System. Annu. Rev. Cell Dev. Biol. 19, 623-647.
Ghysen, A., and Dambly-Chaudière, C. (2007). The lateral line microcosmos. Genes Dev 21, 2118-2130.
Haas, P., and Gilmour, D. (2006). Chemokine signaling mediates self-organizing tissue migration in the zebrafish lateral line. Dev Cell 10, 673-680.
Halloran, M. C., Sato-Maeda, M., Warren, J. T., Su, F., Lele, Z., Krone, P. H., Kuwada, J. Y., and Shoji, W. (2000). Laser-induced gene expression in specific cells of transgenic zebrafish. Development 127, 1953-1960.
Hatzikirou, H., and Deutsch, A. (2008). Cellular automata as microscopic models of cell migration in heterogeneous environments. Curr. Top. Dev. Biol. 81, 401-434.
Keller, E. F., and Segel, L. A. (1971). Model for chemotaxis. Journal of Theoretical Biology 30, 225-234.
Kicheva, A., Pantazis, P., Bollenbach, T., Kalaidzidis, Y., Bittig, T., Jülicher, F., and González-Gaitán, M. (2007). Kinetics of morphogen gradient formation. Science 315, 521-525.
Kimmel, C. B., Ballard, W. W., Kimmel, S. R., Ullmann, B., and Schilling, T. F. (1995). Stages of embryonic development of the zebrafish. Dev Dyn 203, 253-310.
Li, Q., Shirabe, K., and Kuwada, J. (2004). Chemokine signaling regulates sensory cell migration in zebrafish. Dev Biol 269, 123-136.
Marchese, A., and Benovic, J. L. (2001). Agonist-promoted ubiquitination of the G protein-coupled receptor CXCR4 mediates lysosomal sorting. J Biol Chem 276, 45509-45512.
Marchese, A., Raiborg, C., Santini, F., Keen, J. H., Stenmark, H., and Benovic, J. L. (2003). The E3 ubiquitin ligase AIP4 mediates ubiquitination and sorting of the G protein-coupled receptor CXCR4. Dev Cell 5, 709-722.
Mato, J. M., Losada, A., Nanjundiah, V., and Konijn, T. M. (1975). Signal input for a chemotactic response in the cellular slime mold Dictyostelium discoideum. Proc Natl Acad Sci USA 72, 4991-4993.
Minina, S., Reichman-Fried, M., and Raz, E. (2007). Control of receptor internalization, signaling level, and precise arrival at the target in guided cell migration. Curr Biol 17, 1164-1172.
Montell, D. J. (2008). Morphogenetic Cell Movements: Diversity from Modular Mechanical Properties. Science 322, 1502-1505.
Montell, D. J. (2003). Border-cell migration: the race is on. Nat Rev Mol Cell Biol 4, 13-24.
Muller, P., Rogers, K. W., Jordan, B. M., Lee, J. S., Robson, D., Ramanathan, S., and Schier, A. F. (2012). Differential Diffusivity of Nodal and Lefty Underlies a Reaction-Diffusion Patterning System. Science.
Muller, P., Rogers, K. W., Yu, S. R., Brand, M., and Schier, A. F. (2013). Morphogen transport. Development 140, 1621-1638.
Muller, P., and Schier, A. F. (2011). Extracellular movement of signaling molecules. Dev Cell 21, 145-158.
Parent, C. A. (1999). A Cell's Sense of Direction. Science 284, 765-770.
Rajagopal, S., Kim, J., Ahn, S., Craig, S., Lam, C. M., Gerard, N. P., Gerard, C., and Lefkowitz, R. J. (2009). -arrestin- but not G protein-mediated signaling by the “decoy” receptor CXCR7. Proceedings of the National Academy of Sciences 1-5.
Rørth, P. (2007). Collective guidance of collective cell migration. Trends in Cell Biology 17, 575-579.
Rørth, P. (2009). Collective cell migration. Annu. Rev. Cell Dev. Biol. 25, 407-429.
Rørth, P. (2011). Whence directionality: guidance mechanisms in solitary and collective cell migration. Dev Cell 20, 9-18.
Sánchez-Alcañiz, J. A., Haege, S., Mueller, W., Pla, R., Mackay, F., Schulz, S., López-Bendito, G., Stumm, R., and Marin, O. (2011). Cxcr7 controls neuronal migration by regulating chemokine responsiveness. Neuron 69, 77-90.
Swaney, K. F., Huang, C.-H., and Devreotes, P. N. (2010). Eukaryotic Chemotaxis: A Network of Signaling Pathways Controls Motility, Directional Sensing, and Polarity. Annu. Rev. Biophys. 39, 265-289.
Teleman, A. A., and Cohen, S. M. (2000). Dpp gradient formation in the Drosophila wing imaginal disc. Cell 103, 971-980.
Thisse, C., and Thisse, B. (2008). High-resolution in situ hybridization to whole-mount zebrafish embryos. Nature Protocols 3, 59-69.
Tranquillo, R. T., Lauffenburger, D. A., and Zigmond, S. H. (1988). A stochastic model for leukocyte random motility and chemotaxis based on receptor binding fluctuations. The Journal of Cell Biology 106, 303-309.
Valentin, G., Haas, P., and Gilmour, D. (2007). The chemokine SDF1a coordinates tissue migration through the spatially restricted activation of Cxcr7 and Cxcr4b. Curr Biol 17, 1026-1031.
Veldkamp, C. T. (2005). The monomer-dimer equilibrium of stromal cell-derived factor-1 (CXCL 12) is altered by pH, phosphate, sulfate, and heparin. Protein Science 14, 1071-1081.
Wang, X., He, L., Wu, Y. I., Hahn, K. M., and Montell, D. J. (2010). Light-mediated activation reveals a key role for Rac in collective guidance of cell movement in vivo. Nat Cell Biol 12, 591-597.
Wartlick, O., Kicheva, A., and Gonzalez-Gaitan, M. (2009). Morphogen Gradient Formation. Cold Spring Harbor Perspectives in Biology 1, a001255-a001255.
Weber, M., Hauschild, R., Schwarz, J., Moussion, C., de Vries, I., Legler, D. F., Luther, S. A., Bollenbach, T., and Sixt, M. (2013). Interstitial Dendritic Cell Guidance by Haptotactic Chemokine Gradients. Science 339, 328-332.
Yu, S. R., Burkhardt, M., Nowak, M., Ries, J., Petrá\check{s} ek, Z., Scholpp, S., Schwille, P., and Brand, M. (2009). Fgf8 morphogen gradient forms by a source-sink mechanism with freely diffusing molecules. Nature 1-5. (1975). Mathematics of diffusion (Oxford University Press).

### SUPPLEMENTAL REFERENCES

Carslaw, H. S., and Jaeger, J. C. (1959). Conduction of heat in solids (Clarendon Press).
Deen, W. (1998). Analysis of Transport Phenomenon (Oxford University Press).
Doetsch, G. (1971). Guide to the Applications of the Laplace and Z-Transforms (Van Nostrand-Reinhold).
Doitsidou, M., Reichman-Fried, M., Stebler, J., Köprunner, M., Dörries, J., Meyer, D., Esguerra, C. V., Leung, T., and Raz, E. (2002). Guidance of primordial germ cell migration by the chemokine SDF-1. Cell 111, 647-659.
Haas, P., and Gilmour, D. (2006). Chemokine signaling mediates self-organizing tissue migration in the zebrafish lateral line. Dev Cell 10, 673-680.
Howard, J., Grill, S. W., and Bois, J. S. (2011). Turing's next steps: the mechanochemical basis of morphogenesis. Nat Rev Mol Cell Biol 12, 392-398.
Kettleborough, R. N. W., Busch-Nentwich, E. M., Harvey, S. A., Dooley, C. M., de Bruijn, E., van Eeden, F., Sealy, I., White, R. J., Herd, C., Nijman, U., et al. (2013). A systematic genome-wide analysis of zebrafish protein-coding gene function. Nature 1-6.
Kicheva, A., Pantazis, P., Bollenbach, T., Kalaidzidis, Y., Bittig, T., Jülicher, F., and González-Gaitán, M. (2007). Kinetics of morphogen gradient formation. Science 315, 521-525.
Kimmel, C. B., Ballard, W. W., Kimmel, S. R., Ullmann, B., and Schilling, T. F. (1995). Stages of embryonic development of the zebrafish. Dev Dyn 203, 253-310.
Knaut, H., Blader, P., Strähle, U., and Schier, A. F. (2005). Assembly of trigeminal sensory ganglia by chemokine signaling. Neuron 47, 653-666.
Knaut, H., Werz, C., Geisler, R., Nüsslein-Volhard, C., and Tübingen 2000 Screen Consortium (2003). A zebrafish homologue of the chemokine receptor Cxcr4 is a germ-cell guidance receptor. Nature 421, 279-282.
Kollmar, R., Nakamura, S. K., Kappler, J. A., and Hudspeth, A. J. (2001). Expression and phylogeny of claudins in vertebrate primordia. Proc Natl Acad Sci USA 98, 10196-10201.
Lewellis, S. W., Nagelberg, D., Subedi, A., Staton, A., LeBlanc, M., Giraldez, A., and Knaut, H. (2013). Precise SDF1-mediated cell guidance is achieved through ligand clearance and microRNA-mediated decay. The Journal of Cell Biology 200, 337-355.
Valentin, G., Haas, P., and Gilmour, D. (2007). The chemokine SDF1a coordinates tissue migration through the spatially restricted activation of Cxcr7 and Cxcr4b. Curr Biol 17, 1026-1031.
Veldkamp, C. T. (2005). The monomer-dimer equilibrium of stromal cell-derived factor-1 (CXCL 12) is altered by pH, phosphate, sulfate, and heparin. Protein Science 14, 1071-1081.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. An expression vector comprising a single transcriptional unit, wherein the single transcriptional unit comprises a first nucleic acid sequence encoding a G protein coupled receptor operably linked to a first detectable marker and a second nucleic acid sequence encoding a second detectable marker operably linked to a membrane localizing domain, wherein the first nucleic acid sequence is operably linked to the second nucleic acid sequence via an internal ribosomal entry site (IRES) or nucleic acid sequences encoding a viral 2A peptide sequence and the first and second detectable markers emit distinct detectable signals.

2. The expression vector of claim 1, wherein the G protein coupled receptor is CXCR1 (MIM 146929), CXCR2 (MIM 146928), CXCR3 (MIM 300574), CXCR4 (MIM 162643), CXCR5 (MIM 601613), CXCR6 (MIM 605163), CXCR7 (MIM 610376), CCR1 (MIM 601159), CCR2 (MIM 601267), CCR3 (MIM 601268), CCR4 (MIM 604836), CCR5 (MIM 601373), CCR6 (MIM 601835), CCR7 (MIM 600242), CCR8 (MIM 601834), CCR9 (MIM 604738), CCR10 (MIM 600240), CX3CR1 (MIM 601470), GPR15 (MIM 601166), FPR (MIM 136537), D6 (MIM 602648), DARC/Duffy (MIM 613665), CCX-CKR (MIM 606065), PAR1 (MIM 187930), PAR2 (MIM 600933), PAR3 (MIM 601919), PAR4 (MIM 602779), ADRB1 (MIM 109630), ADRB2 (MIM 109690), ADRB3 (MIM 109691), ADRA1B (MIM 104220), ADRA1D (MIM 104219), ADRA1A (MIM 104221), ADRA2A (MIM 104210), S1PR1 (MIM 601974), S1PR2 (MIM 605111), S1PR3 (MIM 601965), S1PR4 (MIM 603751) or S1PR5 (MIM 605146).

3. The expression vector of claim 1, wherein the first nucleic acid sequence is operably linked to the second nucleic acid sequence via an internal ribosomal entry site (IRES) and the IRES is a viral IRES or a cellular IRES.

4. The expression vector of claim 3, wherein the viral IRES is the EMCV-R IRES, CrPV IGR IRES, HCV type 1a IRES, FMDV type C IRES, HAV HM175 IRES, PV type 1 Mahoney IRES, PV type 3 Leon IRES, AEV IRES, CSFV IRES, or ERAV 245-961 IRES.

5. The expression vector of claim 3, wherein the cellular IRES is the APAF-1 IRES, BCL2 IRES, ELH IRES, FMR1 IRES, HSP70, Kv1.4 1.2 IRES, LEF1 IRES, MTG8a IRES, MYB IRES, or URE2 IRES.

6. The expression vector of claim 1, wherein the membrane localizing domain is a CaaX motif, the lyn tyrosine kinase transmembrane domain, or the CD8 transmembrane domain.

7. The expression vector of claim 1, wherein the expression vector comprises a single promoter that drives expression of the first and second nucleic acid sequences.

8-10. (canceled)

11. A method for screening to identify a modulator of a G protein coupled receptor, the method comprising providing a plurality of cells comprising the expression vector of claim 1; contacting the plurality of cells with at least one candidate compound or agent; and measuring detectable signals of the first and second detectable markers in the presence or absence of the at least one candidate compound or agent to determine if the presence of the at least one candidate compound or agent causes a change in the detectable signals of the first and second detectable markers, wherein a change in detectable signals in the presence of the at least one candidate compound or agent identifies the at least one candidate compound or agent as a G protein coupled receptor modulator.

12. The method of claim 11, wherein the change in detectable signals reflects relocalization of the first detectable signal, wherein the relocalization is detected by a reduction in co-localization of detectable signals of the first and second detectable markers.

13. The method of claim 12, wherein the first detectable signal relocalizes from plasma membranes to early endosomes or endosomal vesicles of the plurality of cells.

14. The method of claim 11, wherein the first and second detectable markers are fluorescent markers.

15-17. (canceled)

18. The method of claim 11, further comprising adding a known ligand or agonist of the G protein coupled receptor to facilitate screening to identify an antagonist of the G protein coupled receptor.

19. The method of claim 11, wherein the at least one candidate compound or agent is a component of a library of compounds or agents or a component of a tissue extract.

20-21. (canceled)

22. A method for screening to identify a modulator of a G protein coupled receptor, the method comprising:
providing a plurality of cells comprising an expression vector or cassette comprising a first and a second nucleic acid sequence, wherein the first nucleic acid sequence encodes a G protein coupled receptor operably linked to a first detectable marker and the second nucleic acid sequence encodes a second detectable marker operably linked to a membrane localizing domain, wherein the first nucleic acid sequence is operably linked to the second nucleic acid sequence via an internal ribosomal entry site (IRES) or nucleic acid sequences encoding a viral 2A peptide sequence and the first and second detectable markers emit distinct detectable signals;
contacting the plurality of cells with at least one candidate compound or agent; and
measuring detectable signals from the first and second detectable markers in the presence or absence of the at least one candidate compound or agent to determine if the presence of the at least one candidate compound or agent causes a change in the detectable signals from the first and second detectable markers, wherein a change in detectable signals in the presence of the at least one candidate compound or agent identifies the at least one candidate compound or agent as a G protein coupled receptor modulator.

23. The method of claim 22, wherein the G protein coupled receptor is CXCR1 (MIM 146929), CXCR2 (MIM 146928), CXCR3 (MIM 300574), CXCR4 (MIM 162643), CXCR5 (MIM 601613), CXCR6 (MIM 605163), CXCR7 (MIM 610376), CCR1 (MIM 601159), CCR2 (MIM 601267), CCR3 (MIM 601268), CCR4 (MIM 604836), CCR5 (MIM 601373), CCR6 (MIM 601835), CCR7 (MIM 600242), CCR8 (MIM 601834), CCR9 (MIM 604738), CCR10 (MIM 600240), CX3CR1 (MIM 601470), GPR15 (MIM 601166), FPR (MIM 136537), D6 (MIM 602648), DARC/Duffy (MIM 613665), CCX-CKR (MIM 606065), PAR1 (MIM 187930), PAR2 (MIM 600933), PAR3 (MIM 601919), PAR4 (MIM 602779), ADRB1 (MIM 109630), ADRB2 (MIM 109690), ADRB3 (MIM 109691), ADRA1B (MIM 104220), ADRA1D (MIM 104219), ADRA1A (MIM 104221), ADRA2A (MIM 104210), S1PR1 (MIM 601974), S1PR2 (MIM 605111), S1PR3 (MIM 601965), S1PR4 (MIM 603751) or S1PR5 (MIM 605146).

24. The method of claim 22, wherein the first nucleic acid sequence is operably linked to the second nucleic acid sequence via an internal ribosomal entry site (IRES) and the IRES is a viral IRES or a cellular IRES.

25. The method of claim 24, wherein the viral IRES is the EMCV-R IRES, CrPV IGR IRES, HCV type 1a IRES, FMDV type C IRES, HAV HM175 IRES, PV type 1 Mahoney IRES, PV type 3 Leon IRES, AEV IRES, CSFV IRES, or ERAV 245-961 IRES.

26. The method of claim 24, wherein the cellular IRES is the APAF-1 IRES, BCL2 IRES, ELH IRES, FMR1 IRES, HSP70, Kv1.4 1.2 IRES, LEF1 IRES, MTG8a IRES, MYB IRES, or URE2 IRES.

27. (canceled)

28. The method of claim 22, wherein the change in detectable signals reflects relocalization of the first detectable signal, wherein the relocalization is detected by a reduction in co-localization of detectable signals of the first and second detectable markers.

29-38. (canceled)

39. The method of claim 22, wherein the expression vector comprises a single promoter that drives expression of the first and second nucleic acid sequences.

40. (canceled)
